(19) United States
(12) Patent Application Publication
(10) Pub. No.: US 2021/0147472 A1
(43) Pub. Date: May 20, 2021

(54) SOLID FORMS AND FORMULATIONS COMPRISING A GLUCOCORTICOID RECEPTOR ANTAGONIST AND USES THEREOF

(71) Applicant: ORIC Pharmaceuticals, Inc., South San Francisco, CA (US)

(72) Inventors: Valeria R. FANTIN, Burlingame, CA (US); Qiuping YE, Foster City, CA (US); Belinda DE LEON, Daly City, CA (US); Daqing SUN, Foster City, CA (US); Sunil V. MHASKAR, South San Francisco, CA (US)

(21) Appl. No.: 17/045,413

(22) PCT Filed: Apr. 11, 2019
(86) PCT No.: PCT/US2019/027062

§ 371 Date: Oct. 5, 2020

Related U.S. Application Data
(60) Provisional application No. 62/656,249, filed on Apr. 11, 2018.

(51) Int. Cl. C07J 41/00; A61K 9/48; A61K 9/00; A61K 45/06

(52) U.S. Cl. CPC C07J 41/0077; A61K 9/4858; A61K 9/4808; C07B 2200/13; A61K 9/0053; A61K 45/06; A61K 9/4825

(57) ABSTRACT

The present invention relates generally to formulations and methods for treating cancer. Provided herein are formulations comprising substituted steroidal derivatives. The subject formulations are useful for the treatment of cancer.

### CROSS REFERENCE

This application claims the benefit of U.S. Application Ser. No. 62/656,249 filed Apr. 11, 2018, which is hereby incorporated by reference in its entirety.

### BACKGROUND OF THE INVENTION

A need exists in the art for an effective treatment of cancer and neoplastic disease.

### SUMMARY OF THE INVENTION

Provided herein are formulations comprising substituted steroidal derivative compounds. The subject formulations comprise a compound that is useful as an inhibitor of glucocorticoid receptors (GR). Furthermore, the subject formulations are useful for the treatment of cancer, such as prostate cancer, breast cancer, lung cancer, ovarian cancer, melanoma, bladder cancer, renal cancer, or hepatocellular carcinoma.

Efficient synthetic procedures are often required for large scale pilot plant syntheses of chemical compounds. Provided herein are certain scalable processes and methods for the synthesis of Compound 1.

Disclosed herein is a lipid-based formulation comprising:

(a) a lipid; and

(b) a compound of Formula (I), or a pharmaceutically acceptable salt thereof:

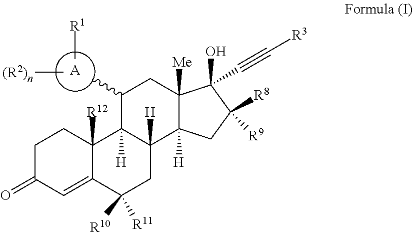

wherein
ring A is a heteroaryl or aryl;
R¹ is —NR^{4a}R^{5a};
each R² is independently —NR⁴R⁵, halo, —OR⁶, —OH, optionally substituted alkyl, or haloalkyl; R³ is optionally substituted C₂₋₈ alkyl, halo, haloalkyl, optionally substituted cycloalkyl, optionally substituted cycloalkylalkyl, optionally substituted heterocycloalkyl, optionally substituted heterocycloalkylalkyl, optionally substituted heteroalkyl, optionally substituted aryl, optionally substituted heteroaryl, —Si(R⁶)₃, —OR⁶, or —S(O)₂R⁷;
R^{4a} is C₂₋₈ alkyl, optionally substituted cycloalkyl, optionally substituted aryl, optionally substituted heterocycloalkyl, or optionally substituted heteroaryl;
R^{5a} is —H, optionally substituted alkyl, or haloalkyl;
or R^{4a} and R^{5a} are taken together with the N atom to which they are attached to form an optionally substituted heterocycloalkyl;
R⁴ and R⁵ are each independently —H, optionally substituted alkyl, or haloalkyl;
or R⁴ and R⁵ are taken together with the N atom to which they are attached to form an optionally substituted heterocycloalkyl;
each R⁶ is independently optionally substituted alkyl or haloalkyl;
R⁷ is optionally substituted alkyl or haloalkyl;
R⁸ and R⁹ are each independently —H, optionally substituted alkyl, haloalkyl, or halo;
R¹⁰ and R¹¹ are each independently —H, optionally substituted alkyl, halo, or haloalkyl;
R¹² is hydrogen, optionally substituted alkyl, haloalkyl, hydroxy, or halo;
n is 0, 1, or 2.

In some embodiments of a lipid-based formulation, R¹² is C₁₋₆ alkyl or hydrogen. In some embodiments of a lipid-based formulation, R¹² is methyl. In some embodiments of a lipid-based formulation, R¹² is H. In some embodiments of a lipid-based formulation, wherein ring A is phenyl. In some embodiments of a lipid-based formulation, R^{4a} is C₂₋₈ alkyl. In some embodiments of a lipid-based formulation, R^{4a} is C₃₋₆ alkyl. In some embodiments of a lipid-based formulation, R^{4a} is C₂₋₄ alkyl. In some embodiments of a lipid-based formulation, R^{4a} is ethyl, i-propyl, or t-butyl. In some embodiments of a lipid-based formulation, R^{5a} is —H, optionally substituted alkyl, or haloalkyl. In some embodiments of a lipid-based formulation, R^{5a} is —H or alkyl. In some embodiments of a lipid-based formulation, R^{5a} is C₁₋₆ alkyl. In some embodiments of a lipid-based formulation, n is 0 or 1. In some embodiments of a lipid-based formulation, each R² is independently halo. In some embodiments of a lipid-based formulation, R³ is optionally substituted C₂₋₈ alkyl, haloalkyl, or optionally substituted cycloalkyl. In some embodiments of a lipid-based formulation, R³ is C₄₋₈ alkyl. In some embodiments of a lipid-based formulation, R⁸ and R⁹ are —H. In some embodiments of a lipid-based formulation, R¹⁰ and R¹¹ are each —H. In some embodiments of a lipid-based formulation, the compound has the structure of Formula (Ia):

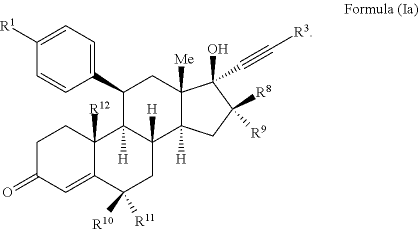

In some embodiments of a lipid-based formulation, the compound is:

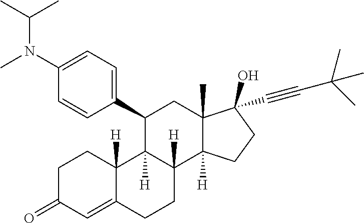

or a pharmaceutically acceptable salt thereof.

In some embodiments of a lipid-based formulation, the compound of Formula (I) is in the form of an HCl salt. In some embodiments of a lipid-based formulation, the compound of Formula (I) is in the form of a free base. In some embodiments of a lipid-based formulation, the lipid is propylene glycol monocaprylate (Capryol®), caprylic acid, capric acid, lauric acid, myristic acid, palmitic acid, stearic acid, oleic acid, ethyl oleate, soybean oil, glyceryl caprylate/caprate (Campul®) glyceryl behenate (Compritol® 888 ATO), glyceryl palmitostearate (Precirol® ATO 5), glyceryl monostearate (Geleol™), glyceryl monolinoleate (Maisine™ 35-1), glyceryl monooleate, (Peceol™), medium-chain triglycerides (Labrafac™ Lipophile WL1349), propylene glycol monolaurate (Lauroglycol™ 90), oleoyl macrogol-6 glycerides (Labrafil® M1944CS), polyglyceryl-3 dioleate (Plurol Oleique® CC 497), diethylene glycol monoethyl ether (Transcutol® HP), or any combinations thereof. In some embodiments of a lipid-based formulation, the lipid-based formulation further comprises a surfactant. In some embodiments of a lipid-based formulation, the surfactant is macroglycerol ricinoleate (Kolliphor EL® or Cremophor EL®), caprylocaproyl polyoxyl-8 glyceride (Labrasol®), lauroyl polyoxyl-6 glycerides (Labrafil® M 2130 CS), lauroyl polyoxyl-32 glyceride (Gelucire® 44/14), polyethylene glycol monostearate (Gelucire® 48/16), polyoxyethylene hydrogenated castor oil 60 (HCO-60), polysorbate 80 (Tween®-80), polyethylene glycol sorbitan monolaurate (Tween®-20), polyoxyethylene sorbitan trioleate (Tween®-85), polyoxyethyelene glyceryl trioleate (tagot-TO), sorbitan monooleate (Span®-80), sorbitan monolaurate (Span®-20), or any combinations thereof. In some embodiments of a lipid-based formulation, the lipid-based formulation further comprises an antioxidant. In some embodiments of a lipid-based formulation, the antioxidant is α-tocopherol, ascorbyl palmitate, butylated hydroxyanisole (BHA), butylated hydroxytoluene (BHT), sodium metabisulfite, potassium metabisulfite, propyl gallate, ascorbic acid, monothioglycerol, propionic acid, sodium ascorbate, sodium bisulfite, sodium sulfite, and cysteine (CYS), or any combinations thereof. In some embodiments of a lipid-based formulation, the antioxidant is α-tocopherol, ascorbyl palmitate, or any combinations thereof. In some embodiments of a lipid-based formulation, the antioxidant is α-tocopherol. In some embodiments of a lipid-based formulation, the antioxidant is ascorbyl palmitate. In some embodiments of a lipid-based formulation, the lipid-based formulation further comprises a solvent. In some embodiments of a lipid-based formulation, the solvent is polyethylene glycol, propylene glycol, glycerin, diethylene glycol monoethyl ether (Transcutol®), triacetin (Kollisolv® GTA), medium chain triglycerides (Miglyol® 812N), or any combinations thereof. In some embodiments of a lipid-based formulation, the formulation is encapsulated. In some embodiments of a lipid-based formulation, the formulation is encapsulate is a gelatin capsule. In some embodiments of a lipid-based formulation, the amount of compound of Formula (I) or its pharmaceutically acceptable salt, in the capsule is between about 10 mg and about 100 mg. In some embodiments of a lipid-based formulation, the amount of the compound of Formula (I) or its pharmaceutically acceptable salt, in the capsule is between about 20 mg and about 80 mg. In some embodiments of a lipid-based formulation, the amount of the compound of Formula (I) or its pharmaceutically acceptable salt, in the capsule is between about 40 mg and about 60 mg. In some embodiments of a lipid-based formulation, the amount of the compound of Formula (I) or its pharmaceutically acceptable salt, in the capsule is between about 60 mg and about 100 mg. In some embodiments of a lipid-based formulation, the amount of the compound of Formula (I) or its pharmaceutically acceptable salt, in the capsule is about 50 mg. In some embodiments of a lipid-based formulation, the amount of the compound of Formula (I) or its pharmaceutically acceptable salt, in the capsule is about 80 mg. In some embodiments of a lipid-based formulation, the amount of lipid is between about 500 mg and about 900 mg. In some embodiments of a lipid-based formulation, the amount of lipid is between about 700 mg and about 800 mg. In some embodiments of a lipid-based formulation, the amount of lipid is between about 600 mg and about 700 mg. In some embodiments of a lipid-based formulation, the amount of surfactant is between about 100 mg and about 500 mg. In some embodiments of a lipid-based formulation, the amount of surfactant is between about 100 mg and about 200 mg. In some embodiments of a lipid-based formulation, the lipid-based formulation comprises caprylic acid. In some embodiments of a lipid-based formulation, the amount of caprylic acid is about 750 mg. In some embodiments of a lipid-based formulation, the amount of caprylic acid is about 735 mg. In some embodiments of a lipid-based formulation, the lipid-based formulation comprises propylene glycol monocaprylate (Capryol®) and macroglycerol ricinoleate (Kolliphor EL® or Cremophor EL®). In some embodiments of a lipid-based formulation, the amount of propylene glycol monocaprylate (Capryol®) is about 676 mg and the amount of macroglycerol ricinoleate (Kolliphor EL® or Cremophor EL®) is about 174 mg. In some embodiments of a lipid-based formulation, the lipid-based formulation comprises α-tocopherol and ascorbyl palmitate. In some embodiments of a lipid-based formulation, the amount of α-tocopherol is about 4.1 mg and the amount of ascorbyl palmitate is about 0.25 mg. In some embodiments of a lipid-based formulation, the lipid-based formulation forms a self-emulsifying drug delivery system (SEDDS) in an aqueous solution. In some embodiments of a lipid-based formulation, the formulation is stable at about 5° C.±3° C. for at least 7 days. In some embodiments of a lipid-based formulation, the formulation is stable at about 25° C.±5° C. for at least 7 days.

Also disclosed herein is a powder for reconstitution comprising a compound of Formula (I), or a pharmaceutically acceptable salt thereof:

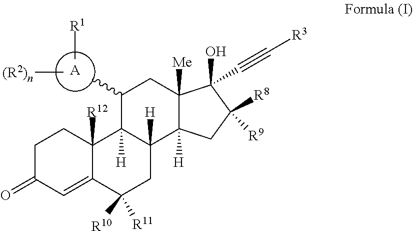

wherein
ring A is a heteroaryl or aryl;
R¹ is —NR^{4a}R^{5a};
each R² is independently —NR⁴R⁵, halo, —OR⁶, —OH, optionally substituted alkyl, or haloalkyl;
R³ is optionally substituted C₂₋₈ alkyl, halo, haloalkyl, optionally substituted cycloalkyl, optionally substituted cycloalkylalkyl, optionally substituted heterocycloalkyl, optionally substituted heterocycloalkylalkyl, optionally substituted heteroalkyl, optionally substituted aryl, optionally substituted heteroaryl, —Si(R⁶)₃, —OR⁶, or —S(O)₂R⁷;
R^{4a} is C₂₋₈ alkyl, optionally substituted cycloalkyl, optionally substituted aryl, optionally substituted heterocycloalkyl, or optionally substituted heteroaryl;
R^{5a} is —H, optionally substituted alkyl, or haloalkyl;
or R^{4a} and R^{5a} are taken together with the N atom to which they are attached to form an optionally substituted heterocycloalkyl;
R⁴ and R⁵ are each independently —H, optionally substituted alkyl, or haloalkyl;
or R⁴ and R⁵ are taken together with the N atom to which they are attached to form an optionally substituted heterocycloalkyl;
each R⁶ is independently optionally substituted alkyl or haloalkyl;
R⁷ is optionally substituted alkyl or haloalkyl;
R⁸ and R⁹ are each independently —H, optionally substituted alkyl, haloalkyl, or halo;
R¹⁰ and R¹¹ are each independently —H, optionally substituted alkyl, halo, or haloalkyl;
R¹² is hydrogen, optionally substituted alkyl, haloalkyl, hydroxy, or halo;
n is 0, 1, or 2.

In some embodiments of a powder for reconstitution, R¹² is C₁₋₆ alkyl or hydrogen. In some embodiments of a powder for reconstitution, R¹² is methyl. In some embodiments of a powder for reconstitution, R¹² is H. In some embodiments of a powder for reconstitution, ring A is phenyl. In some embodiments of a powder for reconstitution, R^{4a} is C₂₋₈ alkyl. In some embodiments of a powder for reconstitution, R^{4a} is C₃₋₆ alkyl. In some embodiments of a powder for reconstitution, R^{4a} is C₂₋₄ alkyl. In some embodiments of a powder for reconstitution, R^{4a} is ethyl, i-propyl, or t-butyl. In some embodiments of a powder for reconstitution, R^{5a} is —H, optionally substituted alkyl, or haloalkyl. In some embodiments of a powder for reconstitution, R^{5a} is —H or alkyl. In some embodiments of a powder for reconstitution, R^{5a} is C₁₋₆ alkyl. In some embodiments of a powder for reconstitution, n is 0 or 1. In some embodiments of a powder for reconstitution, each R² is independently halo. In some embodiments of a powder for reconstitution, R³ is optionally substituted C₂₋₈ alkyl, haloalkyl, or optionally substituted cycloalkyl. In some embodiments of a powder for reconstitution, R³ is C₄₋₈ alkyl. In some embodiments of a powder for reconstitution, R⁸ and R⁹ are —H. In some embodiments of a powder for reconstitution, R¹⁰ and R¹¹ are each —H. In some embodiments of a powder for reconstitution, the compound has the structure of Formula (Ia):

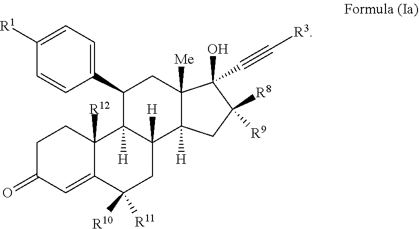

In some embodiments of a powder for reconstitution, the compound is:

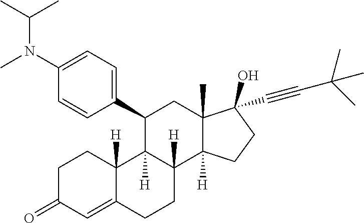

or a pharmaceutically acceptable salt thereof.

In some embodiments of a powder for reconstitution, the compound of Formula (I) is in the form of an HCl salt. In some embodiments of a powder for reconstitution, the compound of Formula (I) is in the form of a free base. In some embodiments of a powder for reconstitution, the powder further comprises a dispersion polymer. In some embodiments of a powder for reconstitution, the dispersion polymer is hydroxypropyl methylcellulose (HPMC), hypromellose acetate succinate (hydroxypropyl methyl cellulose acetate succinate; HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, polyoxyethylene-polyoxypropylene block copolymers, or combinations thereof. In some embodiments of a powder for reconstitution, the dispersion polymer is hydroxypropyl methylcellulose (HPMC). In some embodiments of a powder for reconstitution, the compound of Formula (I) is amorphous. In some embodiments of a powder for reconstitution, the powder is stored in an amber bottle. In some embodiments of a powder for reconstitution, amount of the compound of Formula (I) or its pharmaceutically acceptable salt in the bottle is between about 50 mg and about 1000 mg. In some embodiments of a powder for reconstitution, the amount of the compound of Formula (I) or its pharmaceutically acceptable salt in the bottle is about 100 mg. In some embodiments of a powder for reconstitution, the amount of the compound of Formula (I) or its pharmaceutically acceptable salt in the bottle is about 800 mg. In some embodiments of a powder for reconstitution, the powder is stable at about 5° C.±3° C. for at least 7 days. In some embodiments of a powder for reconstitution, the powder is stable at about 25° C.±5° C. for at least 7 days. In some embodiments of a powder for reconstitution, the powder is reconstituted with a liquid carrier. In some embodiments of a powder for reconstitution, the liquid carrier is an aqueous carrier. In some embodiments of a powder for reconstitution, the liquid carrier comprises sweetening agents, flavoring agents, buffering agents, preservatives, gelling agents, thickening agents, stabilizing agents, or any combination thereof. In some embodiments of a powder for reconstitution, the powder is reconstituted immediately prior to administration.

Also disclosed herein is a process of manufacturing a powder for reconstitution disclosed herein, the process comprising:
(i) Adding a solvent to a vessel;
(ii) Adding the compound of Formula (I) or its pharmaceutically acceptable salt to the vessel;
(iii) Adding a dispersion polymer to the vessel to obtain a first mixture;
(iv) Mixing the first mixture until the compound of Formula (I) or its pharmaceutically acceptable salt and the dispersion polymer are dissolved in the solvent to obtain a first solution;
(v) Dry spraying the first solution to obtain a first solid; and
(vi) Drying the first solid to obtain the powder for reconstitution.

In some embodiment of a process of manufacturing a powder for reconstitution, the solvent comprises water and an alcohol. In some embodiment of a process of manufacturing a powder for reconstitution, the dispersion polymer is hydroxypropyl methylcellulose (HPMC).

Also disclosed herein is a suspension comprising a compound of Formula (I), or a pharmaceutically acceptable salt thereof:

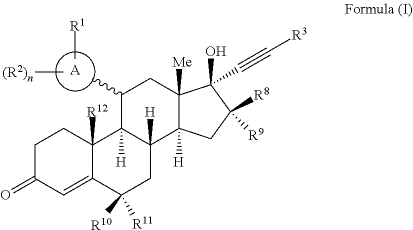

wherein
ring A is a heteroaryl or aryl;
R¹ is —NR^{4a}R^{5a};
each R² is independently —NR⁴R⁵, halo, —OR⁶, —OH, optionally substituted alkyl, or haloalkyl;
R³ is optionally substituted C₂₋₈ alkyl, halo, haloalkyl, optionally substituted cycloalkyl, optionally substituted cycloalkylalkyl, optionally substituted heterocycloalkyl, optionally substituted heterocycloalkylalkyl, optionally substituted heteroalkyl, optionally substituted aryl, optionally substituted heteroaryl, —Si(R⁶)₃, —OR⁶, or —S(O)₂R⁷;
R^{4a} is Cₘ alkyl, optionally substituted cycloalkyl, optionally substituted aryl, optionally substituted heterocycloalkyl, or optionally substituted heteroaryl;
R^{5a} is —H, optionally substituted alkyl, or haloalkyl;
or R^{4a} and R^{5a} are taken together with the N atom to which they are attached to form an optionally substituted heterocycloalkyl;
R⁴ and R⁵ are each independently —H, optionally substituted alkyl, or haloalkyl;
or R⁴ and R⁵ are taken together with the N atom to which they are attached to form an optionally substituted heterocycloalkyl;
each R⁶ is independently optionally substituted alkyl or haloalkyl;
R⁷ is optionally substituted alkyl or haloalkyl;
R⁸ and R⁹ are each independently —H, optionally substituted alkyl, haloalkyl, or halo;
R¹⁰ and R¹¹ are each independently —H, optionally substituted alkyl, halo, or haloalkyl;
R¹² is hydrogen, optionally substituted alkyl, haloalkyl, hydroxy, or halo;
n is 0, 1, or 2.

In some embodiment of a suspension, R¹² is C₁₋₆ alkyl or hydrogen. In some embodiment of a suspension, R¹² is methyl. In some embodiment of a suspension, R¹² is H. In some embodiment of a suspension, ring A is phenyl. In some embodiment of a suspension, R^{4a} is C₂₋₈ alkyl. In some embodiment of a suspension, R^{4a} is C₃₋₆ alkyl. In some embodiment of a suspension, R^{4a} is C₂₋₄ alkyl. In some embodiment of a suspension, R^{4a} is ethyl, i-propyl, or t-butyl. In some embodiment of a suspension, R^{5a} is —H, optionally substituted alkyl, or haloalkyl. In some embodiment of a suspension, R^{5a} is —H or alkyl. In some embodiment of a suspension, R^{5a} is C₁₋₆ alkyl. In some embodiment of a suspension, n is 0 or 1. In some embodiment of a suspension, each R² is independently halo. In some embodiment of a suspension, R³ is optionally substituted C₂₋₈ alkyl, haloalkyl, or optionally substituted cycloalkyl. In some embodiment of a suspension, R³ is C₄₋₈ alkyl. In some embodiment of a suspension, R⁸ and R⁹ are —H. In some embodiment of a suspension, R¹⁰ and R¹¹ are each —H. In some embodiment of a suspension, the compound has the structure of Formula (Ia):

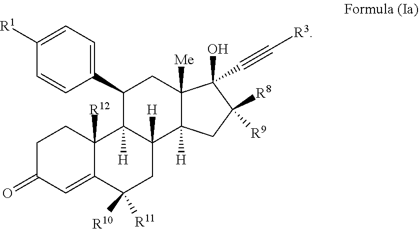

In some embodiment of a suspension, the compound is:

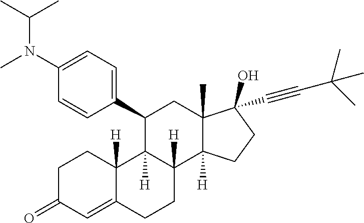

or a pharmaceutically acceptable salt.

In some embodiment of a suspension, the compound of Formula (I) is in the form of an HCl salt. In some embodiment of a suspension, the compound of Formula (I) is in the form of a free base. In some embodiment of a suspension, the concentration of the compound of Formula (I) or its pharmaceutically acceptable salt in the suspension is between about 1 mg/mL and about 20 mg/mL. In some embodiment of a suspension, the concentration of the compound of Formula (I) or its pharmaceutically acceptable salt in the suspension is between about 5 mg/mL and about 20 mg/mL. In some embodiment of a suspension, the concentration of the compound of Formula (I) or its pharmaceutically acceptable salt in the suspension is between about 10 mg/mL and about 20 mg/mL. In some embodiment of a suspension, the concentration of the compound of Formula (I) or its pharmaceutically acceptable salt in the suspension is about 16 mg/mL. In some embodiment of a suspension, the suspension further comprises a liquid carrier. In some embodiment of a suspension, the liquid carrier is an aqueous carrier. In some embodiment of a suspension, the liquid carrier comprises sweetening agents, flavoring agents, buffering agents, preservatives, gelling agents, thickening agents, stabilizing agents, or any combinations thereof. In some embodiment of a suspension, the suspension has a pH between about 3 and about 7. In some embodiment of a suspension, the suspension has a pH between about 3 and about 6. In some embodiment of a suspension, the suspension has a pH between about 3 and about 5. In some embodiment of a suspension, the suspension has a pH between about 3 and about 4. In some embodiment of a suspension, the suspension is stable at about 5° C.±3° C. for at least 24 hours. In some embodiment of a suspension, the suspension is stable at about 25° C.±5° C. for at least 6 hours. In some embodiment of a suspension, the suspension is stable at about −20° C.±5° C. for at least 7 days.

Also disclosed herein is a crystalline compound of Formula (I), or a pharmaceutically acceptable salt thereof:

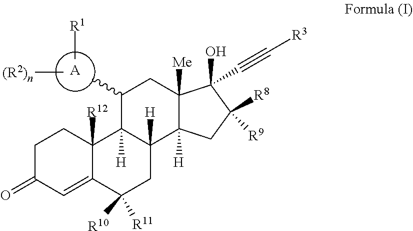

wherein
ring A is a heteroaryl or aryl;
R¹ is —NR^{4a}R^{5a};
each R² is independently —NR⁴R⁵, halo, —OR⁶, —OH, optionally substituted alkyl, or haloalkyl;
R³ is optionally substituted C₂₋₈ alkyl, halo, haloalkyl, optionally substituted cycloalkyl, optionally substituted cycloalkylalkyl, optionally substituted heterocycloalkyl, optionally substituted heterocycloalkylalkyl, optionally substituted heteroalkyl, optionally substituted aryl, optionally substituted heteroaryl, —Si(R⁶)₃, —OR⁶, or —S(O)₂R⁷;
R^{4a} is C₂₋₈ alkyl, optionally substituted cycloalkyl, optionally substituted aryl, optionally substituted heterocycloalkyl, or optionally substituted heteroaryl;
R^{5a} is —H, optionally substituted alkyl, or haloalkyl;
or R^{4a} and R^{5a} are taken together with the N atom to which they are attached to form an optionally substituted heterocycloalkyl;
R⁴ and R⁵ are each independently —H, optionally substituted alkyl, or haloalkyl;
or R⁴ and R⁵ are taken together with the N atom to which they are attached to form an optionally substituted heterocycloalkyl;
each R⁶ is independently optionally substituted alkyl or haloalkyl;
R⁷ is optionally substituted alkyl or haloalkyl;
R⁸ and R⁹ are each independently —H, optionally substituted alkyl, haloalkyl, or halo;
R¹⁰ and R¹¹ are each independently —H, optionally substituted alkyl, halo, or haloalkyl;
R¹² is hydrogen, optionally substituted alkyl, haloalkyl, hydroxy, or halo;
n is 0, 1, or 2.

In some embodiments of crystalline compound of Formula (I), or a pharmaceutically acceptable salt thereof, R¹² is C₁₋₆ alkyl or hydrogen. In some embodiments of crystalline compound of Formula (I), or a pharmaceutically acceptable salt thereof, R¹² is methyl. In some embodiments of crystalline compound of Formula (I), or a pharmaceutically acceptable salt thereof, R¹² is H. In some embodiments of crystalline compound of Formula (I), or a pharmaceutically acceptable salt thereof, ring A is phenyl. In some embodiments of crystalline compound of Formula (I), or a pharmaceutically acceptable salt thereof, R^{4a} is C₂₋₈ alkyl. In some embodiments of crystalline compound of Formula (I), or a pharmaceutically acceptable salt thereof, R^{4a} is C₃₋₆ alkyl. In some embodiments of crystalline compound of Formula (I), or a pharmaceutically acceptable salt thereof, R^{4a} is C₂₋₄ alkyl. In some embodiments of crystalline compound of Formula (I), or a pharmaceutically acceptable salt thereof, R^{4a} is ethyl, i-propyl, or t-butyl. In some embodiments of crystalline compound of Formula (I), or a pharmaceutically acceptable salt thereof, R^{5a} is —H, optionally substituted alkyl, or haloalkyl. In some embodiments of crystalline compound of Formula (I), or a pharmaceutically acceptable salt thereof, R^{5a} is —H or alkyl. In some embodiments of crystalline compound of Formula (I), or a pharmaceutically acceptable salt thereof, R^{5a} is C₁₋₆ alkyl. In some embodiments of crystalline compound of Formula (I), or a pharmaceutically acceptable salt thereof, n is 0 or 1. In some embodiments of crystalline compound of Formula (I), or a pharmaceutically acceptable salt thereof, each R² is independently halo. In some embodiments of crystalline compound of Formula (I), or a pharmaceutically acceptable salt thereof, R³ is optionally substituted C₂₋₈ alkyl, haloalkyl, or optionally substituted cycloalkyl. In some embodiments of crystalline compound of Formula (I), or a pharmaceutically acceptable salt thereof, R³ is C₄₋₈ alkyl. In some embodiments of crystalline compound of Formula (I), or a pharmaceutically acceptable salt thereof, R⁸ and R⁹ are —H. In some embodiments of crystalline compound of Formula (I), or a pharmaceutically acceptable salt thereof, R¹⁰ and R¹¹ are each —H. In some embodiments of crystalline compound of Formula (I), or a pharmaceutically acceptable salt thereof, the compound has the structure of Formula (Ia):

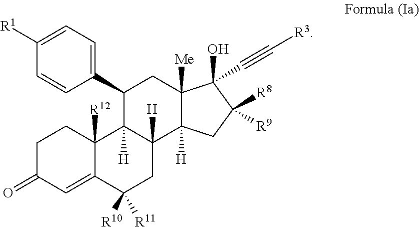

In some embodiments of crystalline compound of Formula (I), or a pharmaceutically acceptable salt thereof, the compound is:

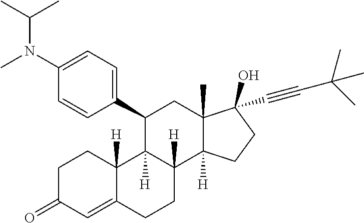

or a pharmaceutically acceptable salt thereof.

In some embodiments of crystalline compound of Formula (I), or a pharmaceutically acceptable salt thereof, the crystalline form has an X-ray powder diffraction (XRPD) pattern substantially the same as shown in FIG. 1. In some embodiments of crystalline compound of Formula (I), or a pharmaceutically acceptable salt thereof, the crystalline form has an X-ray powder diffraction (XRPD) pattern with characteristic peaks at 7.2±0.1° 2-Theta, 15.7±0.1° 2-Theta, 16.6±0.1° 2-Theta, 18.3±0.1° 2-Theta, 19.3±0.1° 2-Theta and 20.1±0.1° 2-Theta. In some embodiments of crystalline compound of Formula (I), or a pharmaceutically acceptable salt thereof, the crystalline form has an X-ray powder diffraction (XRPD) pattern substantially the same as shown in FIG. 3. In some embodiments of crystalline compound of Formula (I), or a pharmaceutically acceptable salt thereof, the crystalline form has an X-ray powder diffraction (XRPD) pattern with characteristic peaks at 7.0±0.1° 2-Theta, 9.2±0.1° 2-Theta, 11.2±0.1° 2-Theta, 14.9±0.1° 2-Theta, 17.2±0.1° 2-Theta, and 19.2±0.1° 2-Theta.

Also disclosed herein is a process for preparing

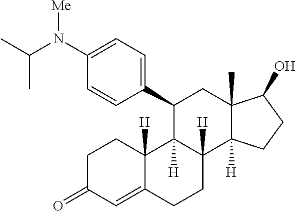

as outlined in Scheme 1.

Also disclosed herein is a process for preparing

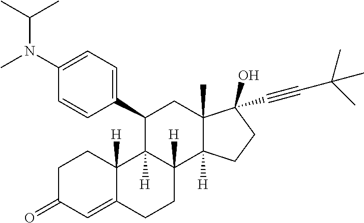

or a pharmaceutically acceptable salt thereof as outlined in Scheme 2.

Also disclosed herein is a method of treating non-small cell lung cancer, triple negative breast cancer, ovarian cancer, melanoma, pancreatic cancer, prostate cancer, castration resistant prostate cancer, renal cancer, melanoma, hepatocellular carcinoma, or bladder cancer, in a subject in need thereof; the method comprising administering a formulation disclosed herein, to the subject in need thereof. In some embodiments of a method of treating, the formulation is administered orally. In some embodiments of a method of treating, the dose of the compound of Formula (I) administered is between about 200 mg and about 800 mg. In some embodiments of a method of treating, the dose of the compound of Formula (I) administered is about 200 mg, about 300 mg, about 400 mg, about 500 mg, about 600 mg, about 700 mg, or about 800 mg. In some embodiments of a method of treating, the formulation is administered once a day. In some embodiments of a method of treating, the formulation is administered twice a day. In some embodiments of a method of treating, the formulation is administered in combination with an additional therapeutic agent. In some embodiments of a method of treating, the additional therapeutic agent is an androgen signaling inhibitor, a chemotherapeutic agent, or immunotherapy. In some embodiments of a method of treating, the androgen receptor signaling inhibitor is 3,3′-diindolylmethane (DIM), abiraterone acetate, apalutamide, darolutamide, bexlosteride, bicalutamide, dutasteride, epristeride, enzalutamide, finasteride, flutamide, izonsteride, ketoconazole, N-butylbenzene-sulfonamide, nilutamide, megestrol, steroidal antiandrogens, or turosteride. In some embodiments of a method of treating, the chemotherapeutic agent is cisplatin, carboplatin, oxaliplatin, etoposide, vincristine, vinblastine, vinorelbine, paclitaxel, docetaxel, nab-paclitaxel, gemcitabine, capecitabine, 5-fluorouracil, doxorubicin, daunorubicin, epirubicin, cyclophosphamide, ifosfamide, camptothecin, topotecan, irinotecan, or pemetrexed. In some embodiments of a method of treating, the chemotherapeutic agent is cisplatin, carboplatin, paclitaxel, docetaxel, nab-paclitaxel, gemcitabine, doxorubicin, camptothecin, topotecan, or pemetrexed. In some embodiments of a method of treating, the immunotherapy is an anti-PD-L1 agent, an anti-PD1 agent, an anti-CTLA-4 agent, a CAR-T cell therapy, an IDO-1 inhibitor, or a cancer vaccine. In some embodiments of a method of treating, the formulation and the additional therapeutic agent are administered concurrently. In some embodiments of a method of treating, the formulation and the additional therapeutic agent are administered intermittently. In some embodiments of a method of treating, the formulation and the additional therapeutic agent are administered in a 21-day therapeutic cycle. In some embodiments of a method of treating, the formulation is administered daily and the additional therapeutic agent is administered on day 1 of a 21-day cycle. In some embodiments of a method of treating, the formulation is administered on days 1-7 and the additional therapeutic agent is administered on day 1 of a 21-day cycle. In some embodiments of a method of treating, the formulation is administered daily and the additional therapeutic agent is administered on day 1, day 8, and day 15 of a 21-day cycle. In some embodiments of a method of treating, the formulation is administered on days 1-7 and the additional therapeutic agent is administered on day 1, day 8, and day 15 of a 21-day cycle. In some embodiments of a method of treating, the formulation is administered for 3 days of each week per 3 week cycle. In some embodiments of a method of treating, the formulation is administered for 4 days of each week per 3 week cycle. In some embodiments of a method of treating, the formulation is administered for 5 days of each week per 3 week cycle. In some embodiments of a method of treating, the formulation is administered for 6 days of each week per 3 week cycle. In some embodiments of a method of treating, the additional therapeutic agent is administered on day 1 of a 21-day cycle. In some embodiments of a method of treating, the additional therapeutic agent is administered on day 1, day 8, and day 15 of a 21-day cycle. In some embodiments of a method of treating, the formulation and additional therapeutic agent are administered for multiple cycles.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the HR-XRPD pattern of (8R,9S,10R,11S,13S,14S,17S)-17-(3,3-dimethylbut-1-yn-1-yl)-17-hydroxy-11-(4-(isopropyl(methyl)amino)phenyl)-13-methyl-1,2,6,7,8,9,10,11,12,13,14,15,16,17-tetradecahydro-3H-cyclopenta[a]phenanthren-3-one (free base Form A).

FIG. 2 depicts Compound 1 exposures in dogs following single administrations of lipid-based and reconstituted powder for reconstitution formulations.

FIG. 3 depicts the HR-XRPD pattern of (8R,9S,10R,11S,13S,14S,17S)-17-(3,3-dimethylbut-1-yn-1-yl)-17-hydroxy-11-(4-(isopropyl(methyl)amino)phenyl)-13-methyl-1,2,6,7,8,9,10,11,12,13,14,15,16,17-tetradecahydro-3H-cyclopenta[a]phenanthren-3-one (hydrochloride monohydrate Form A).

FIG. 4 depicts the exposures of Compound 1 following single administrations of Compound 1 in different formulations.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

“Alkyl” refers to a straight or branched chain hydrocarbon monoradical, which may be fully saturated or unsaturated, having from one to about ten carbon atoms, or from one to six carbon atoms. Examples of saturated hydrocarbon monoradical include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. When the alkyl refers to an unsaturated straight or branched chain hydrocarbon monoradical it is known as an “alkenyl” or an “alkynyl”. The alkenyl may be in either the cis or trans conformation about the double bond(s), and should be understood to include both isomers. Examples of alkenyls include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Examples of alkynyl include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Heterocycloalkyl” refers to a 3- to 24-membered partially or fully saturated ring radical comprising 2 to 23 carbon atoms and from one to 8 heteroatoms selected from nitrogen, oxygen, phosphorous, and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroalkyl” refers to an alkyl group in which one or more skeletal atoms of the alkyl are selected from an atom other than carbon, e.g., oxygen, nitrogen (e.g. —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Unless stated otherwise specifically in the specification, a heteroalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heteroaryl” refers to a 5- to 14-membered ring system radical comprising hydrogen atoms, one to thirteen carbon atoms, one to six heteroatoms selected from nitrogen, oxygen, phosphorous, and sulfur, and at least one aromatic ring. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

“Optional” or “optionally” may be taken to mean that the subsequently described structure, event or circumstance may or may not occur, and that the description includes instances where the events occurs and instances where it does not.

As used herein, the term “therapeutic” means an agent utilized to treat, combat, ameliorate, prevent or improve an unwanted condition or disease of a patient. In some embodiments, a therapeutic agent such as a compound of Formula (I) is directed to the treatment and/or the amelioration of cancers.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with a composition described herein, can include, but is not limited to, providing a composition into or onto the target tissue; providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable”, it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

### Compounds

Provided herein are formulations comprising a compound of Formula (I), or a pharmaceutically acceptable salt thereof:

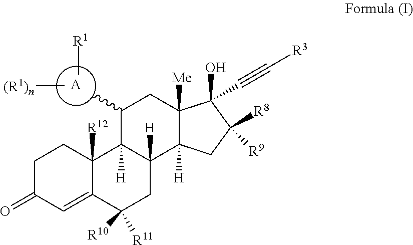

wherein
ring A is a heteroaryl or aryl;
R¹ is —NR^{4a}R^{5a};
each R² is independently —NR⁴R⁵, halo, —OR⁶, —OH, optionally substituted alkyl, or haloalkyl;
R³ is optionally substituted C₂₋₈ alkyl, halo, haloalkyl, optionally substituted cycloalkyl, optionally substituted cycloalkylalkyl, optionally substituted heterocycloalkyl, optionally substituted heterocycloalkylalkyl, optionally substituted heteroalkyl, optionally substituted aryl, optionally substituted heteroaryl, —Si(R⁶)₃, —OR⁶, or —S(O)₂R⁷;
R^{4a} is C₂₋₈ alkyl, optionally substituted cycloalkyl, optionally substituted aryl, optionally substituted heterocycloalkyl, or optionally substituted heteroaryl;
R^{5a} is —H, optionally substituted alkyl, or haloalkyl;
or R^{4a} and R^{5a} are taken together with the N atom to which they are attached to form an optionally substituted heterocycloalkyl;
R⁴ and R⁵ are each independently —H, optionally substituted alkyl, or haloalkyl;
or R⁴ and R⁵ are taken together with the N atom to which they are attached to form an optionally substituted heterocycloalkyl;
each R⁶ is independently optionally substituted alkyl or haloalkyl;
R⁷ is optionally substituted alkyl or haloalkyl;
R⁸ and R⁹ are each independently —H, optionally substituted alkyl, haloalkyl, or halo;
R¹⁰ and R¹¹ are each independently —H, optionally substituted alkyl, halo, or haloalkyl;
R¹² is hydrogen, optionally substituted alkyl, haloalkyl, hydroxy, or halo;
n is 0, 1, or 2.

In some embodiments of compounds of Formula (I), R¹² is C₁₋₆ alkyl or hydrogen. In some embodiments of compounds of Formula (I), R¹² is methyl. In some embodiments of compounds of Formula (I), R¹² is H. In some embodiments of compounds of Formula (I), ring A is phenyl. In some embodiments of compounds of Formula (I), R^{4a} is C₂₋₈ alkyl. In some embodiments of compounds of Formula (I), R^{4a} is C₃₋₆ alkyl. In some embodiments of compounds of Formula (I), R^{4a} is C₂₋₄ alkyl. In some embodiments of compounds of Formula (I), R^{4a} is ethyl, i-propyl, or t-butyl. In some embodiments of compounds of Formula (I), R^{5a} is —H, optionally substituted alkyl, or haloalkyl. In some embodiments of compounds of Formula (I), R^{ea} is —H or alkyl. In some embodiments of compounds of Formula (I), R^{5a} is C₁₋₆ alkyl. In some embodiments of compounds of Formula (I), n is 0 or 1. In some embodiments of compounds of Formula (I), each R² is independently halo. In some embodiments of compounds of Formula (I), R³ is optionally substituted C₂₋₈ alkyl, haloalkyl, or optionally substituted cycloalkyl. In some embodiments of compounds of Formula (I), R³ is C₄₋₈ alkyl. In some embodiments of compounds of Formula (I), R⁸ and R⁹ are —H. In some embodiments of compounds of Formula (I), R¹⁰ and R¹¹ are each —H.

In some embodiments of compounds of Formula (I), the compound has the structure of Formula (Ia):

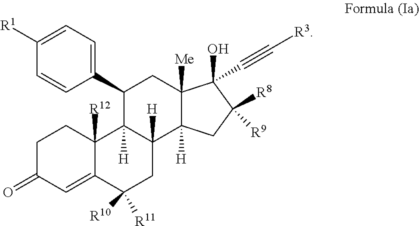

In some embodiments of compounds of Formula (I), the compound is:

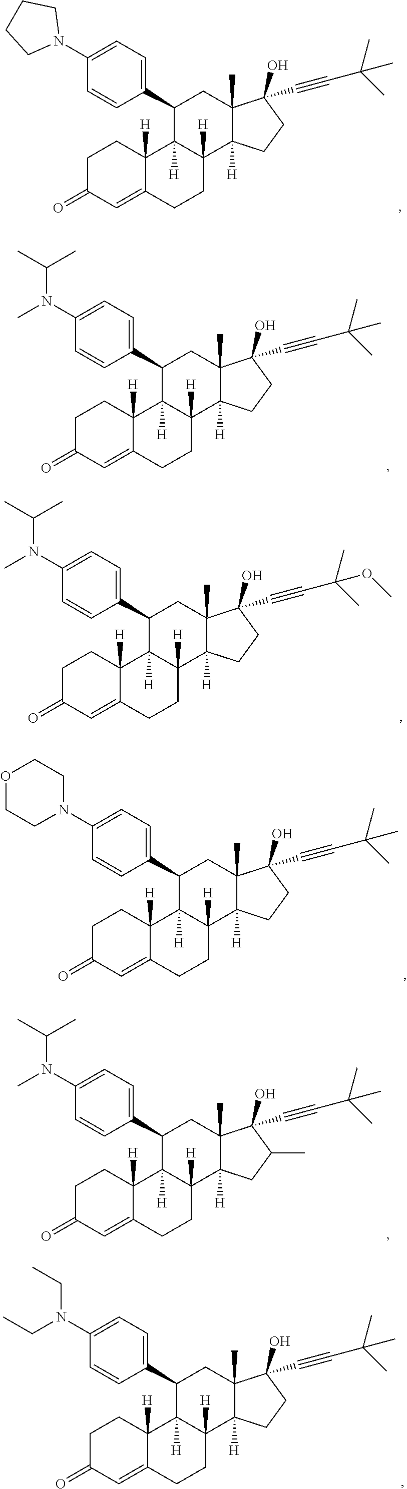

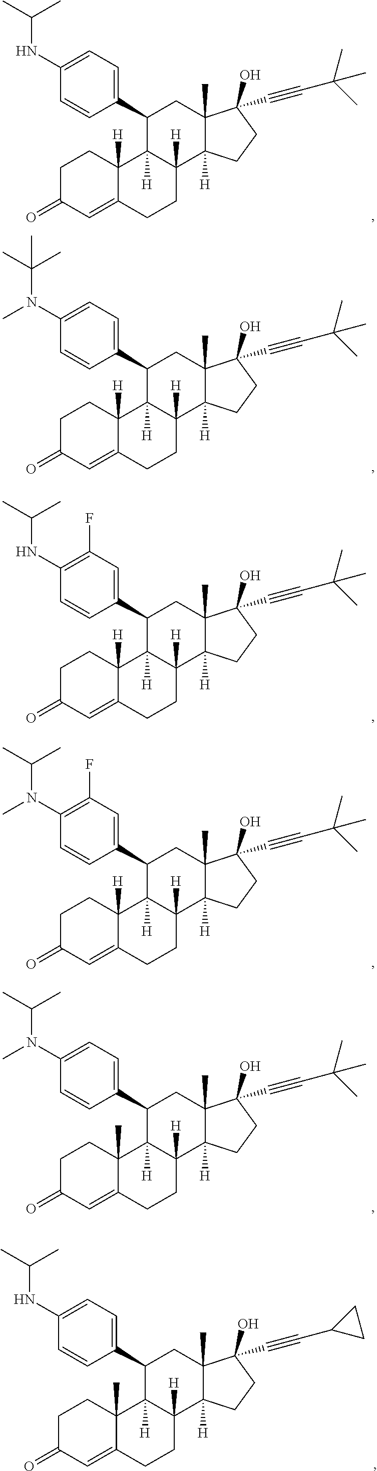

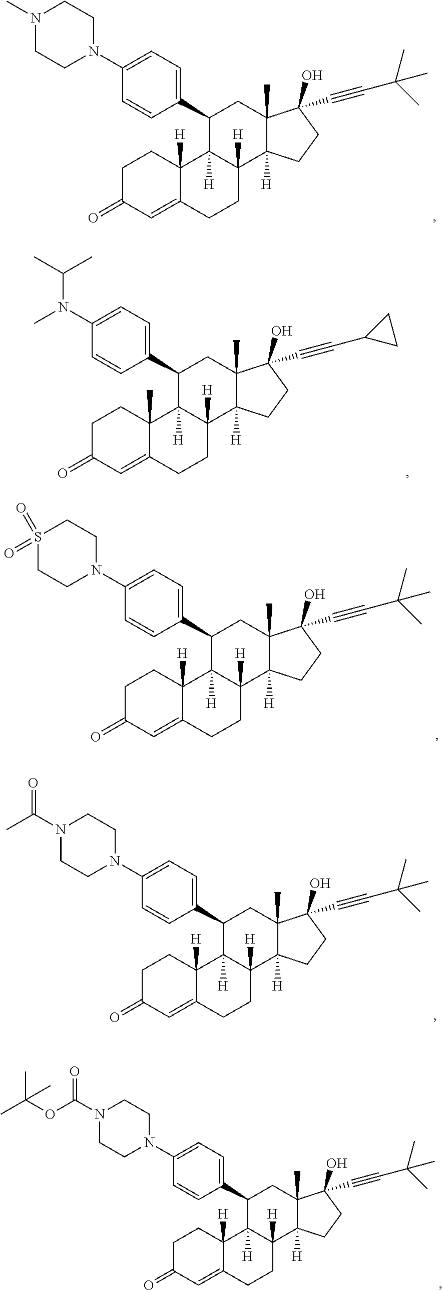

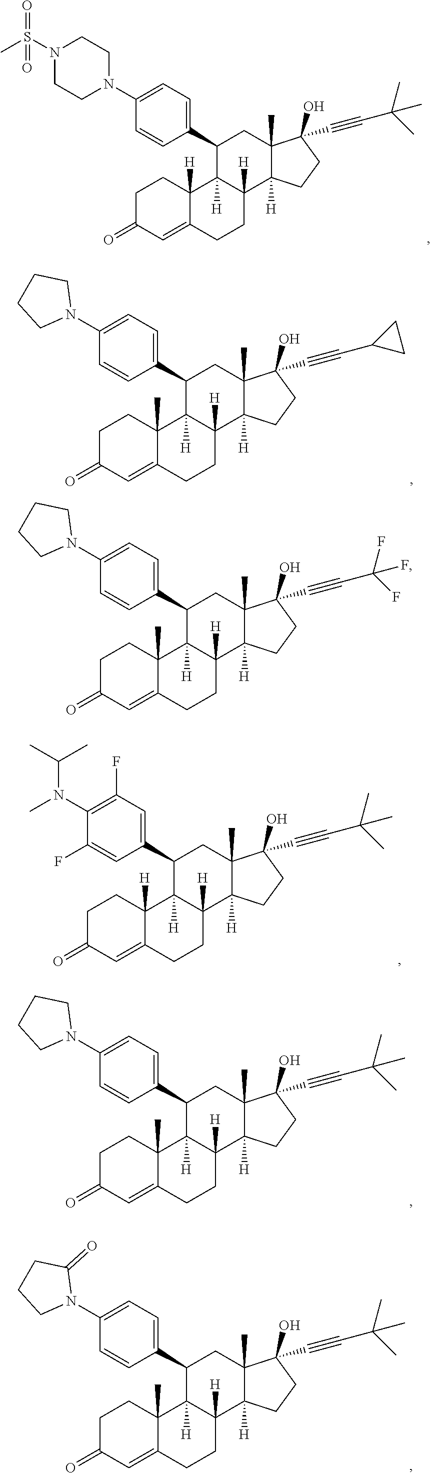

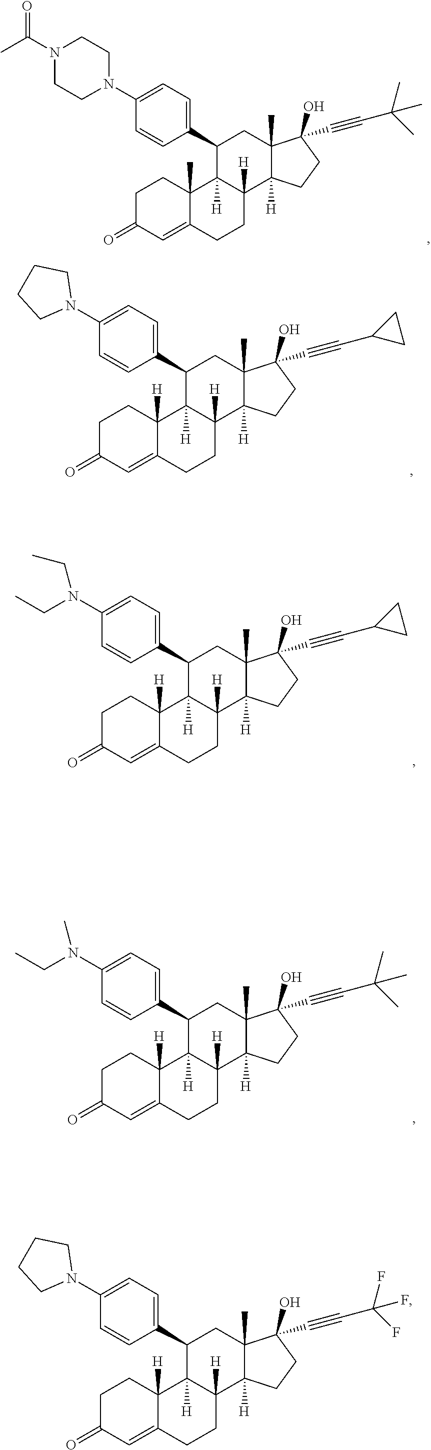

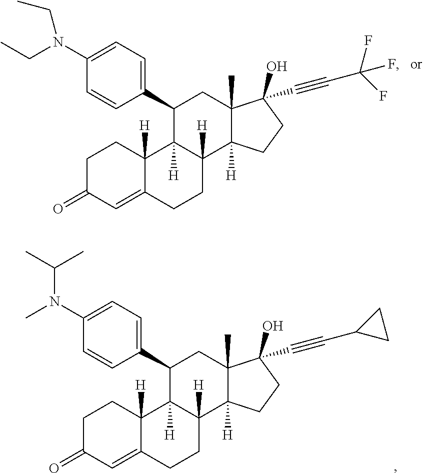

or a pharmaceutically acceptable salt thereof.

In some embodiments of compounds of Formula (I), the compound is Compound 1:

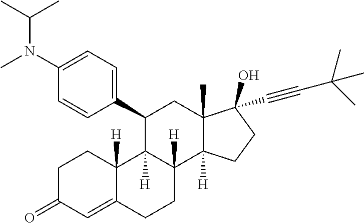

or a pharmaceutically acceptable salt thereof.

### Crystalline Forms

Disclosed herein are formulations comprising a crystalline compound of Formula (I), or a pharmaceutically acceptable salt thereof. In some embodiments, the compound of Formula (I), or a pharmaceutically acceptable salt thereof is Compound 1, or a pharmaceutically acceptable salt thereof. In some embodiments, the formulations described herein comprise crystalline Compound 1 in the form of a free base. In some embodiments, the formulations described herein comprise crystalline Compound 1 in the form of an HCl salt. In some embodiments, the formulations described herein comprise crystalline Compound 1 in the form of an HCl salt hydrate.

### Free Base Form A

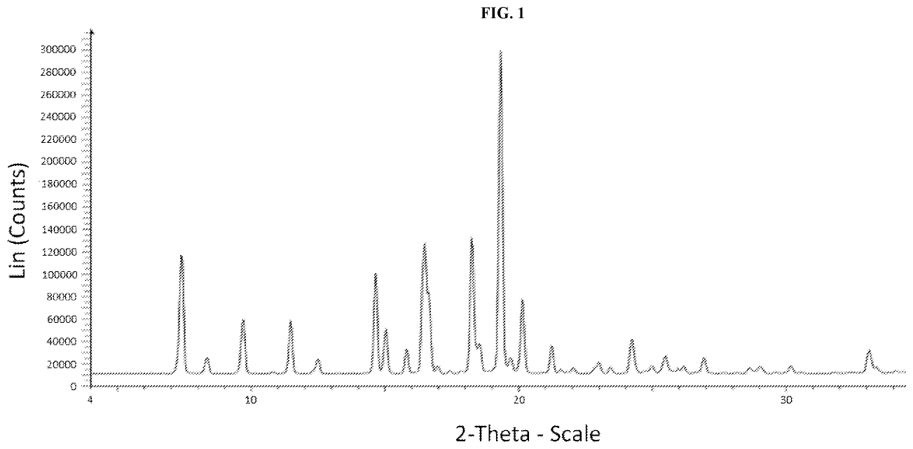

In some embodiments, the formulations described herein comprise crystalline Compound 1 in the form of a free base. In some embodiments, the crystalline form of Compound 1 in the form of a free base is free base Form A. The term “free base polymorph Form A” or “free base Form A” or refers to a crystalline form of (8R,9S,10R,11S,13S,14S,17S)-17-(3,3-dimethylbut-1-yn-1-yl)-17-hydroxy-11-(4-(isopropyl(methyl)amino)phenyl)-13-methyl-1,2,6,7,8,9,10,11,12,13,14,15,16,17-tetradecahydro-3H-cyclopenta[a]phenanthren-3-one (or Compound 1) that exhibits an X-ray powder diffraction pattern substantially the same as that shown in FIG. 1.

In some embodiments, the X-ray powder diffraction (XRPD) pattern of free base Form A is substantially the same as shown in FIG. 1. In some embodiments, free base Form A is characterized by the major peaks of FIG. 1. In some embodiments, the major peaks are the peaks of at least 20%, at least 15% or at least 10% of maximum intensity in the XRPD pattern of FIG. 1.

In some embodiments, free base Form A exhibits an X-ray powder diffraction pattern that includes characteristic peaks at 7.2±0.1° 2-Theta, 15.7±0.1° 2-Theta, 16.6±0.1° 2-Theta, 18.3±0.1° 2-Theta, 19.3±0.1° 2-Theta, and 20.1±0.1° 2-Theta.

In some embodiments, free base Form A has the desired physical properties including crystalline form, melting point, and moisture sorption to be compliant with Good Manufacturing Practices (GMP) for drug manufacturing. In some embodiments, free base Form A is non-hygroscopic. In some instances, this property of decreased hygroscopicity greatly aids in the preparation of solid pharmaceutical dosage forms. In one embodiment, free base Form A is physically stable under humid conditions (e.g., ranging from 10-95 RH).

### HCl Salt Form A

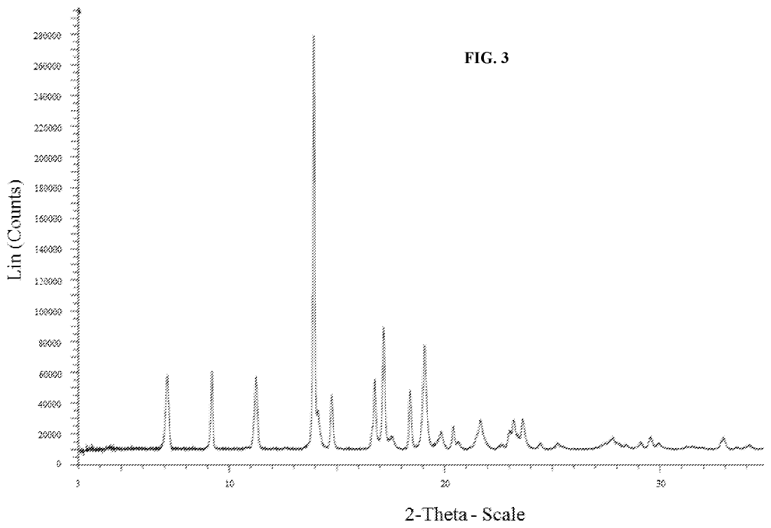

Some embodiments provided herein describe formulations comprising a crystalline Compound 1 hydrochloride monohydrate Form A. In some embodiments, the X-ray powder diffraction (XRPD) pattern of hydrochloride monohydrate Form A is substantially the same as shown in FIG. 3. In some embodiments, hydrochloride monohydrate Form A is characterized by the major peaks of FIG. 3. In some embodiments, the major peaks are the peaks of at least 20%, at least 15% or at least 10% of maximum intensity in the XRPD pattern of FIG. 3.

In some embodiments, hydrochloride monohydrate Form A exhibits an X-ray powder diffraction pattern that includes characteristic peaks at 7.0±0.1° 2-Theta, 9.2±0.1° 2-Theta, 11.2±0.1° 2-Theta, 14.9±0.1° 2-Theta, 17.2±0.1° 2-Theta, and 19.2±0.1° 2-Theta.

In some embodiments, hydrochloride monohydrate Form A has the desired physical properties including crystalline form, melting point, and moisture sorption to be compliant with Good Manufacturing Practices (GMP) for drug manufacturing. In some embodiments, hydrochloride monohydrate Form A is non-hygroscopic. In some instances, this property of decreased hygroscopicity greatly aids in the preparation of solid pharmaceutical dosage forms. In one embodiment, hydrochloride monohydrate Form A is physically stable under humid conditions (e.g., ranging from 10-95 RH).

Also provided herein in some embodiments are formulations comprising a crystalline Compound 1 hydrochloride dehydrate Form A.

### HCl Salt Form B

Some embodiments provided herein describe formulations comprising a crystalline Compound 1 hydrochloride monohydrate Form B.

In some embodiments, hydrochloride monohydrate Form B has the desired physical properties including crystalline form, melting point, and moisture sorption to be compliant with Good Manufacturing Practices (GMP) for drug manufacturing. In some embodiments, hydrochloride monohydrate Form B is non-hygroscopic. In some instances, this property of decreased hygroscopicity greatly aids in the preparation of solid pharmaceutical dosage forms. In one embodiment, hydrochloride monohydrate Form B is physically stable under humid conditions (e.g., ranging from 10-95 RH).

Also provided herein in some embodiments are formulations comprising a crystalline Compound 1 hydrochloride dehydrate Form B.

### Process of Manufacturing

Good manufacturing practices are usually required for large scale manufacture of clinically useful drug candidates. Provided herein are certain processes and methods for the manufacture of compounds of Compound 1, or a pharmaceutically acceptable salt thereof. The processes and methods of syntheses provided herein overcome certain manufacturing drawbacks and allow for synthesis of high purity compounds while reducing waste and/or by-products, and reducing the use of corrosive materials. The improved processes and methods of synthesis of Compound 1 or a pharmaceutically acceptable salt thereof, described herein allow for large-scale production compliant with good manufacturing practice (GMP) guidelines. In some embodiments, the processes and methods of synthesis of Compound 1 or a pharmaceutically acceptable salt thereof, described herein improve the overall yield of Compound 1. In further or additional embodiments, the processes and methods of synthesis of Compound 1 or a pharmaceutically acceptable salt thereof, described herein allow for easier purification of Compound 1.

Disclosed herein is a process for preparing:

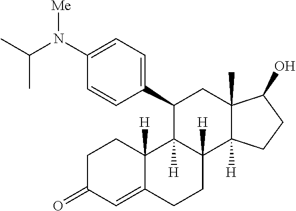

as outlined in Scheme 1.

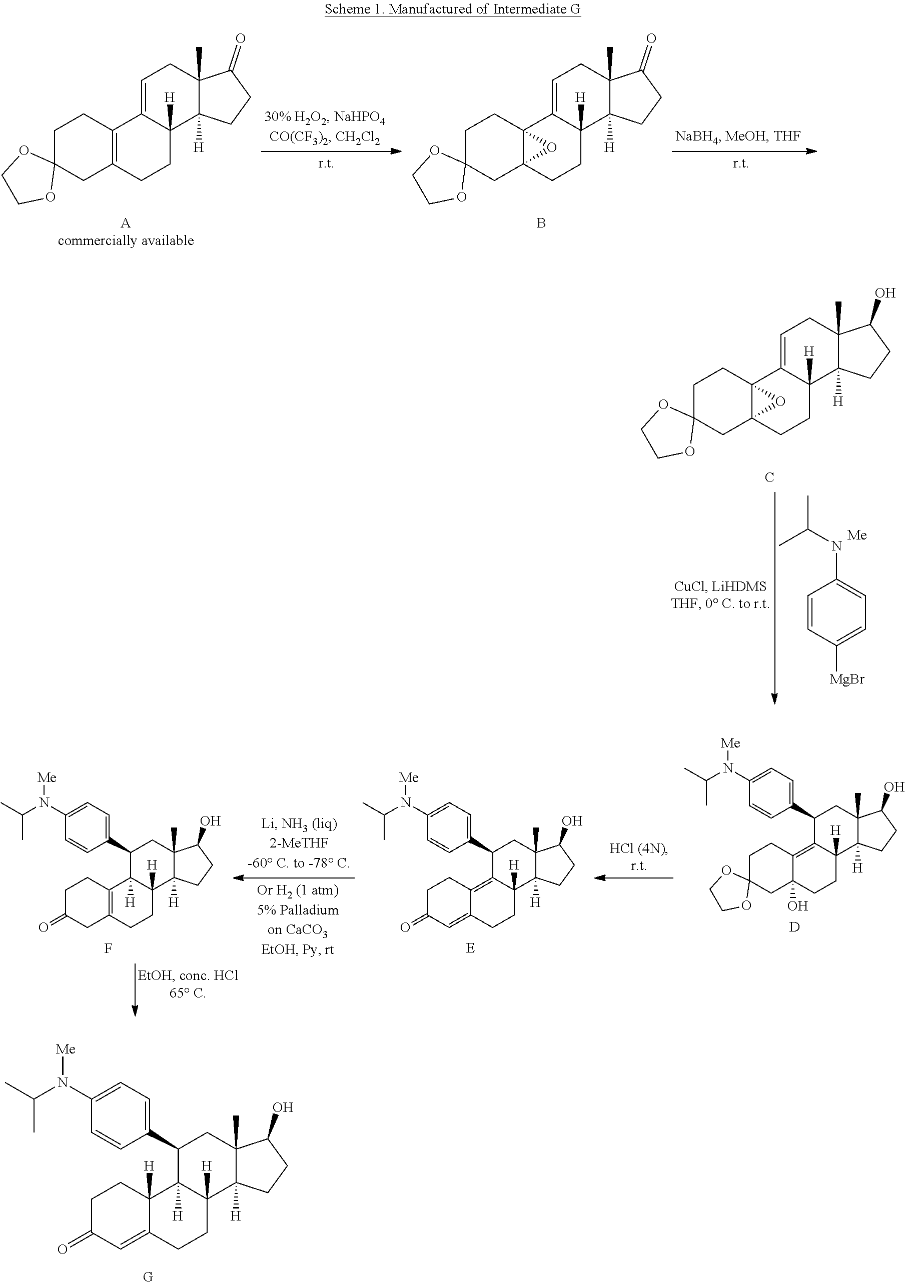

Provided herein in some embodiments is a process to synthesize Intermediate G. In some embodiments, Compound A is transformed to Intermediate G. In some embodiments, Compound A is transformed to Compound B. In some embodiments, Compound B is transformed to Compound C. In further or additional embodiments, Compound C is transformed to Compound D. In further or additional embodiments, Compound D is transformed to Compound E. In further or additional embodiments, Compound E is transformed to Compound F. In some embodiments, Compound F is transformed to Intermediate G.

Disclosed herein is a process for preparing:

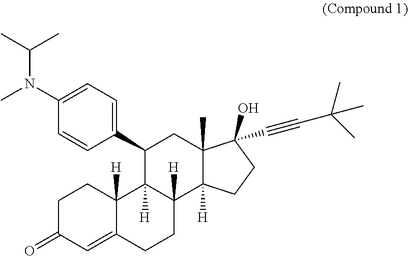

or a pharmaceutically acceptable salt thereof as outlined in Scheme 2. In some embodiments, the process for preparing Compound 1 or a pharmaceutically acceptable salt thereof is described in Schemes 1 and 2.

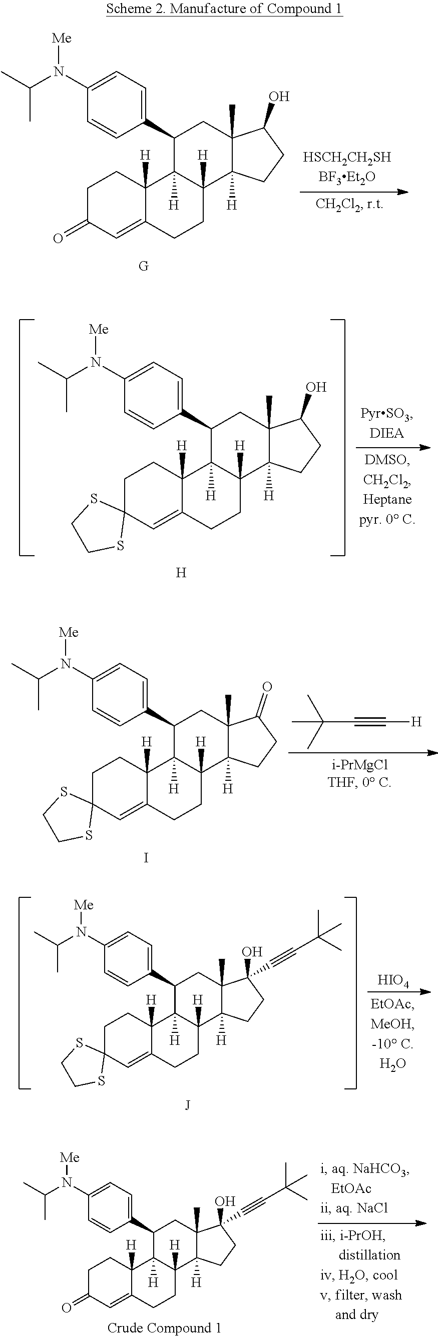

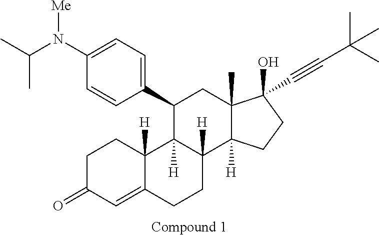

Provided herein in some embodiments is a process to synthesize Compound 1. In some embodiments, Compound A is transformed to Compound 1. In some embodiments, Compound G (Intermediate G) is transformed to Compound 1. In some embodiments, Compound B is transformed to Compound 1. In some embodiments, Compound C is transformed to Compound 1. In some embodiments, Compound D is transformed to Compound 1. In some embodiments, Compound E is transformed to Compound 1. In some embodiments, Compound F is transformed to Compound 1. In some embodiments, Compound I is transformed to Compound 1.

In some embodiments, Compound G (Intermediate G) is transformed to Intermediate H. In some embodiments, Intermediate H is transformed to Compound I. In some embodiments, Compound G (Intermediate G) is transformed to Compound I. In further or additional embodiments, Compound I is transformed to Intermediate H. In further or additional embodiments, Intermediate H is transformed to crude Compound 1. In other embodiments, Compound I is transformed to crude Compound 1. In further or additional embodiments, crude Compound 1 is transformed to Compound 1.

In some embodiments, Compound G (Intermediate G) is transformed to Intermediate H in the presence of 1,2-ethanedithiol, solvent and a Lewis Acid. In some embodiments, Compound G (Intermediate G) is transformed to Intermediate H in the presence of 1,2-ethanedithiol, BF₃.Et₂O and a solvent. Also provided herein in some embodiments is a reaction mixture comprising Compound G, a Lewis acid, and a solvent. Any suitable solvent may be used. In some embodiments, the solvent is water, acetonitrile, DMF, THF, toluene, xylenes, dioxane, butanol, methanol, ethanol, diethyl ether, acetone, hexane, pentane, heptane, ethyl acetate, dichloromethane, dichloroethane, dichlorobenzene, NMP or combinations thereof. In certain embodiments, the Lewis acid is ZnCl₂, FeCl₃, MeAlCl₂, TiCl₄, BF₃, SnCl₄, or AlCl₃. In certain embodiments, the Lewis acid is a BF₃ complex.

In some embodiments, Intermediate H is transformed to Compound I in the presence of pyridine.SO₃ and pyridine in dimethyl sulfoxide and a base. Provided herein in some embodiments is a reaction mixture comprising Intermediate H, pyridine.SO₃, a base, and a solvent. In certain embodiments, the base is N,N-diisopropylethylamine. In some embodiments, the base is ammonia, triethylamine, propylamine, methylamine, dimethylamine, trimethylamine, methyldiethylamine, diisopropylethylamine, aniline, piperidine, pyridine, 1,8-diazabicyclo [5.4.0]undec-7-ene (DBU), or pyrrolidine. Any suitable solvent may be used. In some embodiments, the solvent is water, acetonitrile, DMF, THF, toluene, xylenes, dioxane, butanol, methanol, ethanol, diethyl ether, acetone, hexane, pentane, heptane, ethyl acetate, dichloromethane, dichloroethane, dichlorobenzene, NMP or combinations thereof.

In some embodiments, Compound I is transformed to Intermediate J in the presence of 3,3-dimethyl-1-butyne and an alkylmagnesium halide. In some embodiments, Compound I is transformed to Intermediate J in the presence of 3,3-dimethyl-1-butyne and isopropylmagnesium chloride. In some embodiments, Compound I is transformed to Compound 1 in the presence of 3,3-dimethyl-1-butyne and an alkylmagnesium halide. In some embodiments, Compound I is transformed to Compound 1 in the presence of 3,3-dimethyl-1-butyne and isopropylmagnesium chloride. Also provided herein in some embodiments is a mixture comprising Compound I, 3,3-dimethyl-1-butyne, an alkylmagnesium halide, and a solvent. In certain embodiments, the alkylmagnesium halide is isopropylmagnesium chloride, isopropylmagnesium bromide, or isopropylmagnesium iodide. Any suitable solvent may be used. In some embodiments, the solvent is THF or diethyl ether.

Provided herein in some embodiments is a mixture comprising Compound 1, ethyl acetate and isopropanol.

### Lipid-Based Formulations

Provided herein is a lipid-based formulation comprising:

(a) a lipid; and

(b) a compound of Formula (I), or a pharmaceutically acceptable salt thereof.

In some embodiments, the lipid-based formulation comprises (a) a lipid and (b) Compound 1, or a pharmaceutically acceptable salt thereof. In some embodiments, the lipid-based formulations provided herein improve the solubility of the compounds of Formula (I) or a pharmaceutically acceptable salt thereof. In some embodiments, the lipid-based formulations provided herein improve the bioavailability of the compounds of Formula (I) or a pharmaceutically acceptable salt thereof.

Some embodiments provided herein describe a self-dispersing pharmaceutical compositions, wherein the composition is self-dispersing when added to water and forms an emulsion, microemulsion, or nanoemulsion. In some embodiments, the lipid-based formulations described herein further comprise a surfactant and are in a form of a self-nanoemulsifying drug delivery system (SNEDDS), a self-microemulsifying drug delivery system (SMEDDS), or a self-emulsifying drug delivery system (SEDDS), wherein the lipid-based formulation forms an emulsion in an aqueous solution. In some instances, the lipid-based formulation is “self-emulsifying” and is classified based on the particle sizes that will form upon entry into an aqueous environment, as self-emulsifying drug delivery systems (“SEDDs”) producing particle sizes substantially less than 1 μm, self-microemulsifying drug delivery systems (“SMEDDS”) with smaller particles, and self-nanoemulsifying drug delivery systems (“SNEDDS”) with the smallest particles. In some embodiments, the self-dispersing lipid-based formulations provided herein form SEDDS upon contact with gastric and/or intestinal media in the body, wherein the lipid-based formulation forms an emulsion comprising micelle particles. In some embodiments, the emulsion provides for increased or improved stability of the active agent (e.g., Compound 1) for uptake in the body and/or provide increased or improved surface area for absorption. In some instances, SEDDS provide for enhanced or improved hydrolysis, solubility, bioavailability, absorption, or any combinations thereof of the active agent in vivo. In some embodiments, the SEDDS facilitates the dispersion, dissolution, stability and absorption of the drug, thus improving the bioavailability of said drug. In some embodiments, the self-dispersing lipid-based formulations provided herein improve the solubility of the compounds of Formula (I) or a pharmaceutically acceptable salt thereof. In some embodiments, the self-dispersing lipid-based formulations provided herein improve the bioavailability of the compounds of Formula (I) or a pharmaceutically acceptable salt thereof.

### Lipid

In some embodiments of a lipid-based formulation, the lipid is a long- or medium-chain triglyceride oils with different degrees of saturation.

In some embodiments of a lipid-based formulation, the lipid is propylene glycol monocaprylate (Capryol®), caprylic acid, capric acid, lauric acid, myristic acid, palmitic acid, stearic acid, oleic acid, ethyl oleate, soybean oil, glyceryl caprylate/caprate (Campul®) glyceryl behenate (Compritol® 888 ATO), glyceryl palmitostearate (Precirol® ATO 5), glyceryl monostearate (Geleol™), glyceryl monolinoleate (Maisine™ 35-1), glyceryl monooleate, (Peceol™), medium-chain triglycerides (Labrafac™ Lipophile WL1349), propylene glycol monolaurate (Lauroglycol™ 90), oleoyl macrogol-6 glycerides (Labrafil® M1944CS), polyglyceryl-3 dioleate (Plurol Oleique® CC 497), diethylene glycol monoethyl ether (Transcutol® HP), or any combinations thereof. In some embodiments of a lipid-based formulation, the lipid is propylene glycol monocaprylate (Capryol®) or caprylic acid. In some embodiments of a lipid-based formulation, the lipid is propylene glycol monocaprylate (Capryol®). In some embodiments of a lipid-based formulation, the lipid is caprylic acid.

### Surfactant

In some embodiments of a lipid-based formulation, the lipid-based formulation further comprises a surfactant.

In some embodiments of a lipid-based formulation, the surfactant is macroglycerol ricinoleate (Kolliphor EL® or Cremophor EL®), caprylocaproyl polyoxyl-8 glyceride (Labrasol®), lauroyl polyoxyl-6 glycerides (Labrafil® M 2130 CS), lauroyl polyoxyl-32 glyceride (Gelucire® 44/14), polyethylene glycol monostearate (Gelucire® 48/16), polyoxyethylene hydrogenated castor oil 60 (HCO-60), polysorbate 80 (Tween®-80), polyethylene glycol sorbitan monolaurate (Tween®-20), polyoxyethylene sorbitan trioleate (Tween®-85), polyoxyethyelene glyceryl trioleate (tagot-TO), sorbitan monooleate (Span®-80), sorbitan monolaurate (Span®-20), or any combinations thereof.

In some embodiments of a lipid-based formulation, the surfactant is macroglycerol ricinoleate (Kolliphor EL® or Cremophor EL®), caprylocaproyl polyoxyl-8 glyceride (Labrasol®), Lauroyl polyoxyl-32 glyceride (Gelucire®44/14), polyoxyethylene hydrogenated castor oil 60 (HCO-60), polysorbate 80 (Tween®-80), polyoxyethylene sorbitan trioleate (Tween®-85), polyoxyethyelene glyceryl trioleate (tagot-TO), or any combinations thereof. In some embodiments of a lipid-based formulation, the surfactant is macroglycerol ricinoleate (Kolliphor EL® or Cremophor EL®).

In some embodiments of a lipid-based formulation, the formulation comprises propylene glycol monocaprylate (Capryol®) and macroglycerol ricinoleate (Kolliphor EL® or Cremophor EL®). In some embodiments of a lipid-based formulation, the lipid-based formulation forms a self-emulsifying drug delivery system (SEDDS) in an aqueous solution.

### Antioxidant

In some embodiments of a lipid-based formulation, the lipid-based formulation further comprises an antioxidant. In some embodiments of a lipid-based formulation, the antioxidant is α-tocopherol, ascorbyl palmitate, butylated hydroxyanisole (BHA), butylated hydroxytoluene (BHT), sodium metabisulfite, potassium metabisulfite, propyl gallate, ascorbic acid, monothioglycerol, propionic acid, sodium ascorbate, sodium bisulfite, sodium sulfite, and cysteine (CYS), or any combinations thereof. In some embodiments of a lipid-based formulation, the antioxidant is α-tocopherol, ascorbyl palmitate, or any combinations thereof. In some embodiments of a lipid-based formulation, the antioxidant is α-tocopherol. In some embodiments of a lipid-based formulation, the antioxidant is ascorbyl palmitate.

### Solvent

In some embodiments of a lipid-based formulation, the lipid-based formulation further comprises a solvent. In some embodiments of a lipid-based formulation, the solvent is polyethylene glycol, propylene glycol, glycerin, diethylene glycol monoethyl ether (Transcutol®), triacetin (Kollisolv® GTA), medium chain triglycerides (Miglyol® 812N), or any combinations thereof.

### Capsule

In some embodiments of a lipid-based formulation, the formulation is encapsulated.

In some embodiments, the lipid-based formulation is encapsulated into discrete units. In some embodiments, the lipid-based formulation described herein is enclosed in a capsule.

In some embodiments, the capsule is formed using materials which include, but are not limited to, natural or synthetic gelatin, pectin, casein, collagen, protein, modified starch, polyvinylpyrrolidone, acrylic polymers, cellulose derivatives, or combinations thereof. In some embodiments, the capsule is coated. In some embodiments, the coating covering the capsule includes, but is not limited to, immediate release coatings, protective coatings, enteric or delayed release coatings, sustained release coatings, barrier coatings, seal coatings, or combinations thereof. In some embodiments, a capsule herein is hard or soft. In some embodiments, the capsule is seamless. In some embodiments, the shape and size of the capsule also vary. Examples of capsule shapes include, but are not limited to, round, oval, tubular, oblong, twist off, or a non-standard shape. The size of the capsule may vary according to the volume of the lipid-based formulation. In some embodiments, the size of the capsule is adjusted based on the volume of the lipid-based formulation. Hard or soft gelatin capsules may be manufactured in accordance with conventional methods as a single body unit comprising the standard capsule shape. A single-body soft gelatin capsule typically may be provided, for example, in sizes from 3 to 22 minims (1 minims being equal to 0.0616 ml) and in shapes of oval, oblong or others. The gelatin capsule may also be manufactured in accordance with conventional methods, for example, as a two-piece hard gelatin capsule, sealed or unsealed, typically in standard shape and various standard sizes, conventionally designated as (000), (00), (0), (1), (2), (3), (4), and (5). The largest number corresponds to the smallest size.

### Dosage in Capsule

In some embodiments of a lipid-based formulation, the amount of compound of Formula (I) or its pharmaceutically acceptable salt (e.g., Compound 1 or its pharmaceutically acceptable salt), in the capsule is between about 10 mg and about 100 mg.

In some embodiments of a lipid-based formulation, the amount of the compound of Formula (I) or its pharmaceutically acceptable salt (e.g., Compound 1 or its pharmaceutically acceptable salt), in the capsule is between about 20 mg and about 80 mg.

In some embodiments of a lipid-based formulation, the amount of the compound of Formula (I) or its pharmaceutically acceptable salt (e.g., Compound 1 or its pharmaceutically acceptable salt), in the capsule is between about 40 mg and about 60 mg.

In some embodiments of a lipid-based formulation, the amount of the compound of Formula (I) or its pharmaceutically acceptable salt (e.g., Compound 1 or its pharmaceutically acceptable salt), in the capsule is between about 60 mg and about 100 mg.

In some embodiments of a lipid-based formulation, the amount of compound of Formula (I) or its pharmaceutically acceptable salt (e.g., Compound 1 or its pharmaceutically acceptable salt), in the capsule is about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg.

In some embodiments of a lipid-based formulation, the amount of the compound of Formula (I) or its pharmaceutically acceptable salt (e.g., Compound 1 or its pharmaceutically acceptable salt), in the capsule is about 50 mg.

In some embodiments of a lipid-based formulation, the amount of the compound of Formula (I) or its pharmaceutically acceptable salt (e.g., Compound 1 or its pharmaceutically acceptable salt), in the capsule is about 60 mg.

In some embodiments of a lipid-based formulation, the amount of the compound of Formula (I) or its pharmaceutically acceptable salt (e.g., Compound 1 or its pharmaceutically acceptable salt), in the capsule is about 70 mg.

In some embodiments of a lipid-based formulation, the amount of the compound of Formula (I) or its pharmaceutically acceptable salt (e.g., Compound 1 or its pharmaceutically acceptable salt), in the capsule is about 80 mg.

In some embodiments of a lipid-based formulation, the amount of the compound of Formula (I) or its pharmaceutically acceptable salt (e.g., Compound 1 or its pharmaceutically acceptable salt), in the capsule is about 90 mg.

In some embodiments of a lipid-based formulation, the amount of the compound of Formula (I) or its pharmaceutically acceptable salt (e.g., Compound 1 or its pharmaceutically acceptable salt), in the capsule is about 100 mg.

In some embodiments of a lipid-based formulation, the amount of lipid in the capsule is between about 100 mg and about 1000 mg. In some embodiments of a lipid-based formulation, the amount of lipid in the capsule is about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, or about 1000 mg. In some embodiments of a lipid-based formulation, the amount of lipid in the capsule is between about 500 mg and about 900 mg. In some embodiments of a lipid-based formulation, the amount of lipid in the capsule is between about 700 mg and about 800 mg. In some embodiments of a lipid-based formulation, the amount of lipid in the capsule is between about 600 mg and about 700 mg. In some embodiments of a lipid-based formulation, the amount of lipid in the capsule is about 676 mg. In some embodiments of a lipid-based formulation, the amount of lipid in the capsule is about 750 mg. In some embodiments of a lipid-based formulation, the amount of lipid in the capsule is about 735 mg.

In some embodiments of a lipid-based formulation, the amount of surfactant in the capsule is between about 100 mg and about 500 mg. In some embodiments of a lipid-based formulation, the amount of surfactant in the capsule is about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In some embodiments of a lipid-based formulation, the amount of surfactant in the capsule is between about 100 mg and about 200 mg. In some embodiments of a lipid-based formulation, the amount of surfactant in the capsule is about 174 mg.

In some embodiments of a lipid-based formulation, the amount of antioxidant in the capsule is between about 0.1 mg and about 10 mg. In some embodiments of a lipid-based formulation, the amount of antioxidant in the capsule is about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg. In some embodiments of a lipid-based formulation, the amount of antioxidant in the capsule is between about 0.1 mg and about 5 mg. In some embodiments of a lipid-based formulation, the amount of antioxidant in the capsule is between about 0.1 mg and about 1 mg. In some embodiments of a lipid-based formulation, the amount of antioxidant in the capsule is between about 0.1 mg and about 0.5 mg. In some embodiments of a lipid-based formulation, the amount of antioxidant in the capsule is between about 1 mg and about 5 mg. In some embodiments of a lipid-based formulation, the amount of antioxidant in the capsule is between about 3 mg and about 5 mg. In some embodiments of a lipid-based formulation, the amount of antioxidant in the capsule is about 0.25 mg. In some embodiments of a lipid-based formulation, the amount of antioxidant in the capsule is about 4.1 mg.

### Powder for Reconstitution

Provided herein is a powder for reconstitution comprising a compound of Formula (I), or a pharmaceutically acceptable salt thereof. In some embodiments, the powder for reconstitution comprises Compound 1, or a pharmaceutically acceptable salt thereof.

### Excipients

In some embodiments, the powder for reconstitution described herein comprises additional excipients including, but not limited, to dispersion polymers, buffering agents, glidants, preservatives, sweeteners, flavoring agents, coloring agents, and thickeners. Additional excipients such as bulking agents, tonicity agents, and chelating agents are within the scope of the embodiments.

In some embodiments, the powder for reconstitution described herein comprises a dispersion polymer. Dispersion polymer are selected from hydroxypropyl methylcellulose (HPMC), hypromellose acetate succinate (hydroxypropyl methyl cellulose acetate succinate; HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, polyoxyethylene-polyoxypropylene block copolymers, and combinations thereof. In some embodiments, the dispersion polymer is hydroxypropyl methylcellulose (HPMC).

In some embodiments, the powder for reconstitution described herein comprises a buffering agent. Buffering agents maintain the pH when the powder compositions are reconstituted into a liquid form. Non-limiting examples of buffering agents include, but are not limited to, sodium bicarbonate, potassium bicarbonate, magnesium hydroxide, magnesium lactate, magnesium glucomate, aluminum hydroxide, aluminum hydroxide/sodium bicarbonate co precipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartarate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts. Some buffering agents also impart effervescent qualities when a powder is reconstituted in a solution.

In some embodiments, the powder for reconstitution described herein comprises a glidant. Glidants are substances that improve flowability of a powder. Suitable glidants include, but are not limited to, calcium phosphate tribasic, calcium silicate, cellulose (powdered), colloidal silicon dioxide, magnesium silicate, magnesium trisilicate, silicon dioxide, starch, talc and the like.

In some embodiments, the powder for reconstitution described herein comprises a preservative. Preservatives include anti-microbials, anti-oxidants, and agents that enhance sterility. Exemplary preservatives include ascorbic acid, ascorbyl palmitate, BHA, BHT, citric acid, erythorbic acid, fumaric acid, malic acid, propyl gallate, sodium ascorbate, sodium bisulfate, sodium metabisulfite, sodium sulfite, parabens (methyl-, ethyl-, butyl-), benzoic acid, potassium sorbate, vanillin, and the like.

In some embodiments, the powder for reconstitution described herein comprises a sweetener. Sweeteners or sweetening agents include any compounds that provide a sweet taste. This includes natural and synthetic sugars, natural and artificial sweeteners, natural extracts and any material that initiates a sweet sensation in a subject. In some embodiments, the powder compositions described herein comprise a sweetener. In other embodiments, sweeteners in liquid form referred to as syrups are used to reconstitute the powder compositions described herein.

Sugars illustratively include glucose, fructose, sucrose, xylitol, tagatose, sucralose, maltitol, isomaltulose, Isomalt™ (hydrogenated isomaltulose), lactitol, sorbitol, mannitol, erythritol, trehalose, maltodextrin, polydextrose, and the like. Other sweeteners illustratively include glycerin, inulin, erythritol, maltol, acesulfame and salts thereof, e.g., acesulfame potassium, alitame, aspartame, neotame, sodium cyclamate, saccharin and salts thereof, e.g., saccharin sodium or saccharin calcium, neohesperidin dihydrochalcone, stevioside, thaumatin, and the like. Sweeteners can be used in the form of crude or refined products such as hydrogenated starch hydrolysates, maltitol syrup, high fructose corn syrup, etc., and as branded products, e.g., Sweet Am™ liquid (Product Code 918.003—propylene glycol, ethyl alcohol, and proprietary artificial flavor combination, Flavors of North America) and Sweet Am™ powder (Product Code 918.005—maltodextrin, sorbitol, and fructose combination and Product Code 918.010—water, propylene glycol, sorbitol, fructose, and proprietary natural and artificial flavor combination, Flavors of North America), ProSweet™ (1-10% proprietary plant/vegetable extract and 90-99% dextrose combination, Viriginia Dare), Maltisweet™ (maltitol solution, Ingredion) and Sorbo™ (sorbitol and sorbitol/xylitol solution, SPI Polyols), Invertose™ (high fructose corn syrup, Ingredion) and Ora-Sweet® sugar-free flavored syrup (Paddock Laboratories, Inc.).

In some embodiments, the powder for reconstitution described herein comprises a flavoring agent to enhance the taste or aroma of the composition in liquid form. Suitable natural or synthetic flavoring agents can be selected from standard reference books, for example Fenaroli's Handbook of Flavor Ingredients, 3rd edition (1995).

In some embodiments, the powder for reconstitution described herein comprises a coloring agent for identity and/or aesthetic purposes of the resultant liquid form. Suitable coloring agents illustratively include FD&C Red No. 3, FD&C Red No. 20, FD&C Red No. 40, FD&C Yellow No. 6, FD&C Blue No. 2, D&C Green No. 5, D&C Orange No. 5, caramel, ferric oxide and mixtures thereof.

In some embodiments, the powder for reconstitution described herein comprises a thickener. Thickeners impart viscosity or weight to the resultant liquid forms from the compositions described herein. Exemplary thickeners include dextrin, cellulose derivatives (ethylcellulose, hydroxyethyl cellulose, methylcellulose, hypromellose, and the like) starches, pectin, polyethylene glycol, polyethylene oxide, trehalose and certain gums (xanthan gum, locust bean gum, etc.).

Additional excipients are contemplated in the powder composition embodiments. These additional excipients are selected based on function and compatibility with the powder compositions described herein and may be found, for example in Remington: The Science and Practice of Pharmacy, Nineteenth Ed (Easton, Pa.: Mack Publishing Company, 1995); Hoover, John E., Remington's Pharmaceutical Sciences, (Easton, Pa.: Mack Publishing Co 1975); Liberman, H. A. and Lachman, L., Eds., Pharmaceutical Dosage Forms (New York, N.Y.: Marcel Decker 1980); and Pharmaceutical Dosage Forms and Drug Delivery Systems, Seventh Ed (Lippincott Williams & Wilkins 1999), herein incorporated by reference in their entirety.

### Dosage

In some embodiments of a powder for reconstitution, the powder is stored in an amber bottle where the powder for reconstitution can be reconstituted.

In some embodiments of a powder for reconstitution, the amount of the compound of Formula (I) or its pharmaceutically acceptable salt (e.g., Compound 1 or its pharmaceutically acceptable salt), in the bottle is between about 50 mg and about 1000 mg.

In some embodiments of a powder for reconstitution, the amount of the compound of Formula (I) or its pharmaceutically acceptable salt (e.g., Compound 1 or its pharmaceutically acceptable salt), in the bottle is 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg. In some embodiments of a powder for reconstitution, the amount of the compound of Formula (I) or its pharmaceutically acceptable salt (e.g., Compound 1 or its pharmaceutically acceptable salt), in the bottle is about 100 mg.

In some embodiments of a powder for reconstitution, the amount of the compound of Formula (I) or its pharmaceutically acceptable salt (e.g., Compound 1 or its pharmaceutically acceptable salt), in the bottle is about 800 mg.

### Liquid Carrier for Reconstitution

In some embodiments of a powder for reconstitution, the powder is reconstituted with a liquid carrier. In some embodiments of a powder for reconstitution, the liquid carrier is an aqueous carrier.

In some embodiments of a powder for reconstitution, the liquid carrier comprises sweetening agents, flavoring agents, buffering agents, preservatives, gelling agents, thickening agents, stabilizing agents, or any combination thereof.

In some embodiments, a syrup is used to reconstitute the powder compositions described herein. In some embodiments, Ora-Sweet® flavored syrup is used to reconstitute the powder compositions described herein. In some embodiments, Ora-Blend® syrup is used to reconstitute the powder compositions described herein.

In some embodiments of a powder for reconstitution, the powder is reconstituted immediately prior to administration. In some embodiments of a powder for reconstitution, the powder is reconstituted 1 hour prior to administration. In some embodiments of a powder for reconstitution, the powder is reconstituted 50 minutes prior to administration. In some embodiments of a powder for reconstitution, the powder is reconstituted 40 minutes prior to administration. In some embodiments of a powder for reconstitution, the powder is reconstituted 30 minutes prior to administration. In some embodiments of a powder for reconstitution, the powder is reconstituted 20 minutes prior to administration. In some embodiments of a powder for reconstitution, the powder is reconstituted 10 minutes prior to administration. In some embodiments of a powder for reconstitution, the powder is reconstituted 5 minutes prior to administration.

### pH of the Reconstituted Formulation

In some embodiments of a powder for reconstitution, the reconstituted formulation has a pH between about 3 and about 9. In some embodiments of a powder for reconstitution, the reconstituted formulation has a pH between about 3 and about 8. In some embodiments of a powder for reconstitution, the reconstituted formulation has a pH between about 3 and about 7. In some embodiments of a powder for reconstitution, the reconstituted formulation has a pH between about 5 and about 8. In some embodiments of a powder for reconstitution, the reconstituted formulation has a pH between about 5 and about 7. In some embodiments of a powder for reconstitution, the reconstituted formulation has a pH between about 3 and about 6. In some embodiments of a powder for reconstitution, the reconstituted formulation has a pH between about 3 and about 5. In some embodiments of a powder for reconstitution, the reconstituted formulation has a pH between about 3 and about 4.

### Preparation of Powder for Reconstitution

Preparation of powder compositions described herein includes any known pharmaceutical method. In one embodiment, the powder for reconstitution described herein are prepared by:
(i) Adding a solvent to a vessel;
(ii) Adding the compound of Formula (I) or its pharmaceutically acceptable salt to the vessel;
(iii) Adding a dispersion polymer to the vessel to obtain a first mixture;
(iv) Mixing the first mixture until the compound of Formula (I) or its pharmaceutically acceptable salt and the dispersion polymer are dissolved in the solvent to obtain a first solution;
(v) Dry spraying the first solution to obtain a first solid; and
(vi) Drying the first solid to obtain the powder for reconstitution.

In some embodiments of a method of preparing the powder for reconstitution, the solvent comprises water and an alcohol.

In some embodiments of a method of preparing the powder for reconstitution, the dispersion polymer is hydroxypropyl methylcellulose (HPMC).

### Suspension

Provided herein is a suspension comprising a compound of Formula (I), or a pharmaceutically acceptable salt thereof. In some embodiments, the suspension comprises Compound 1, or a pharmaceutically acceptable salt thereof.

In some embodiment of a suspension, the concentration of the compound of Formula (I) or its pharmaceutically acceptable salt (e.g., Compound 1, or its pharmaceutically acceptable salt) in the suspension is between about 1 mg/mL and about 20 mg/mL. In some embodiment of a suspension, the concentration of the compound of Formula (I) or its pharmaceutically acceptable salt (e.g., Compound 1, or its pharmaceutically acceptable salt) in the suspension is between about 5 mg/mL and about 20 mg/mL. In some embodiment of a suspension, the concentration of the compound of Formula (I) or its pharmaceutically acceptable salt (e.g., Compound 1, or its pharmaceutically acceptable salt) in the suspension is between about 10 mg/mL and about 20 mg/mL.

In some embodiment of a suspension, the concentration of the compound of Formula (I) or its pharmaceutically acceptable salt (e.g., Compound 1, or its pharmaceutically acceptable salt) in the suspension is about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, or about 20 mg/mL. In some embodiment of a suspension, the concentration of the compound of Formula (I) or its pharmaceutically acceptable salt (e.g., Compound 1, or its pharmaceutically acceptable salt) in the suspension is about 16 mg/mL.

In some embodiment of a suspension, the suspension further comprises a liquid carrier.

In some embodiment of a suspension, the liquid carrier is an aqueous carrier. In some embodiment of a suspension, the liquid carrier comprises sweetening agents, flavoring agents, buffering agents, preservatives, gelling agents, thickening agents, stabilizing agents, or any combinations thereof.

In some embodiments, the liquid carrier is a syrup. In some embodiments, the liquid carrier is Ora-Sweet® flavored syrup. In some embodiments, the liquid carrier is Ora-Blend® syrup.

### pH of the Suspension

In some embodiments of a suspension, the suspension has a pH between about 3 and about 9. In some embodiments of a suspension, the suspension has a pH between about 3 and about 8. In some embodiments of a suspension, the suspension has a pH between about 3 and about 7. In some embodiments of a suspension, the suspension has a pH between about 5 and about 8. In some embodiments of a suspension, the suspension has a pH between about 5 and about 7. In some embodiments of a suspension, the suspension has a pH between about 3 and about 6. In some embodiments of a suspension, the suspension has a pH between about 3 and about 5. In some embodiments of a suspension, the suspension has a pH between about 3 and about 4.

### Stability

The compositions described herein are stable in various storage conditions including refrigerated, ambient and accelerated conditions. Stable as used herein refer to formulations having at least about 95% of the compound of Formula (I) (e.g., Compound 1) and about 5% or less total impurities or related substances at the end of a given storage period (by weight). Stability is assessed by HPLC or any other known testing method (see example 4). In some embodiments, the stable formulations have about 5%, about 4%, about 3%, about 2.5%, about 2%, about 1.5%, about 1%, or about 0.5% total impurities or related substances (by weight). In other embodiments, the stable formulations have about 5% total impurities or related substances (by weight). In yet other embodiments, the stable formulations have about 4% total impurities or related substances (by weight). In yet other embodiments, the stable formulations have about 3% total impurities or related substances (by weight). In yet other embodiments, the stable formulations have about 2% total impurities or related substances (by weight). In yet other embodiments, the stable formulations have about 1% total impurities or related substances (by weight). In further embodiments, the stable formulations have about 95%, about 96%, about 97%, about 98% or about 99% of the compound of Formula (I) (e.g., Compound 1) at the end of a given storage period (by weight).

In some embodiments, the stable formulations have less than about 5%, less than about 4%, less than about 3%, less than about 2.5%, less than about 2%, less than about 1.5%, less than about 1%, or less than about 0.5% total impurities or related substances (by weight). In other embodiments, the stable formulations have less than about 5% total impurities or related substances (by weight). In yet other embodiments, the stable formulations have less than about 4% total impurities or related substances (by weight). In yet other embodiments, the stable formulations have less than about 3% total impurities or related substances (by weight). In yet other embodiments, the stable formulations have less than about 2% total impurities or related substances (by weight). In yet other embodiments, the stable formulations have less than about 1% total impurities or related substances (by weight). In further embodiments, the stable formulations have at least about 95%, at least about 96%, at least about 97%, at least about 98% or at least about 99% of the compound of Formula (I) (e.g., Compound 1) at the end of a given storage period (by weight).

At refrigerated and ambient conditions, the formulations described herein are stable for at least 1 month. At refrigerated and ambient conditions, the formulations described herein are stable for at least 30 days, at least 29 days, at least 28 days, at least 27 days, at least 26 days, at least 25 days, at least 24 days, at least 23 days, at least 22 days, at least 21 days, at least 20 days, at least 19 days, at least 18 days, at least 17 days, at least 16 days, at least 15 days, at least 14 days, at least 13 days, at least 12 days, at least 11 days, at least 10 days, at least 9 days, at least 8 days, at least 7 days, at least 6 days, at least 5 days, at least 4 days, at least 3 days, at least 2 days, or at least 1 day. In some instances, a refrigerated condition is at about 2° C., about 3° C., about 4° C., about 5° C., about 6° C., about 7° C. or about 8° C. In other instances, a refrigerated condition is at about 4° C.

At accelerated conditions, the formulations described herein are stable for at least 1 month. At accelerated conditions, the formulations described herein are stable for at least 30 days, at least 29 days, at least 28 days, at least 27 days, at least 26 days, at least 25 days, at least 24 days, at least 23 days, at least 22 days, at least 21 days, at least 20 days, at least 19 days, at least 18 days, at least 17 days, at least 16 days, at least 15 days, at least 14 days, at least 13 days, at least 12 days, at least 11 days, at least 10 days, at least 9 days, at least 8 days, at least 7 days, at least 6 days, at least 5 days, at least 4 days, at least 3 days, at least 2 days, or at least 1 day. Accelerated conditions include temperature and/or relative humidity (RH) that are above ambient levels (e.g. 25±5° C.; 55±10% RH). In some instances, an accelerated condition is at about 30° C., about 35° C., about 40° C., about 45° C., about 50° C., about 55° C. or about 60° C. In other instances, an accelerated condition is above 65% RH, about 70% RH, about 75% RH or about 80% RH. In further instances, an accelerated condition is about 40° C. or 60° C. at ambient humidity. In yet further instances, an accelerated condition is about 40° C. at 75±5% RH humidity. Ambient conditions include temperature and/or relative humidity (RH) that are at ambient levels (e.g. 25±5° C.; 55±10% RH). In some instances, an ambient condition is at about 20° C., about 21° C., about 22° C., about 23° C., about 24° C., about 25° C., about 26° C., about 27° C., about 28° C., about 29° C., or about 30° C. In other instances, an ambient condition is about 45% RH, about 50% RH, about 55% RH, about 60% RH or about 65% RH. Refrigerated conditions include temperature and/or relative humidity (RH) in typical refrigeration units (e.g., 5±3° C.).

The impurities or related substances are as shown in tables 1a and 1b:

TABLE 1a

| Code | RRT | Structure | Origin |
|---|---|---|---|
| A1 | 1.12 | 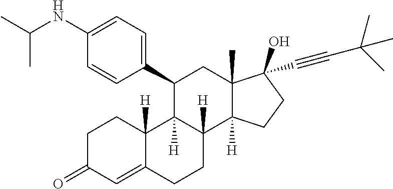 | Process impurity, metabolite |
| A2 | 0.32 | 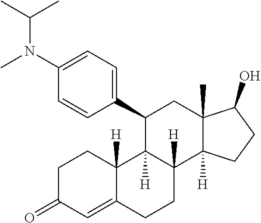 | Process impurity, starting material |
| A3 | 0.42 | 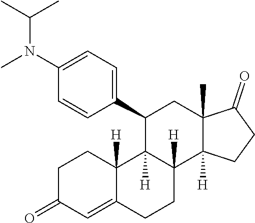 | Process impurity |
| A4 | 0.98 | 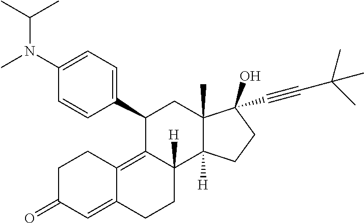 | Process impurity |
| A5 | 1.14 | 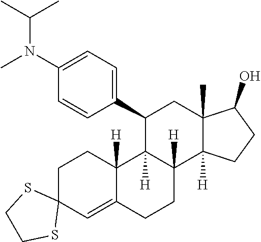 | Process impurity |
| A6 | 1.29 | 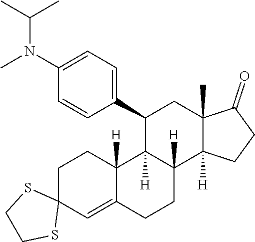 | Process intermediate |
| A7 | 1.61 | 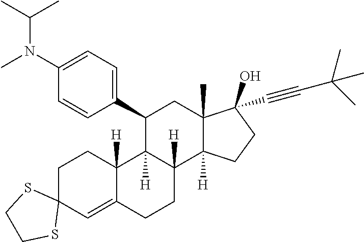 | Process impurity |

TABLE 1b

| Code | RRT | Structure | Origin |
|---|---|---|---|
| B1 | 0.62/0.63 | 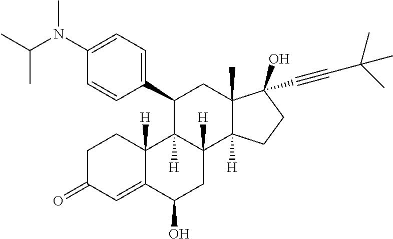 | Degradation product |
| B2 | 0.81 | 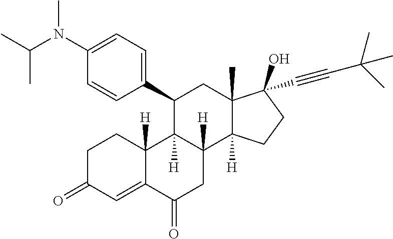 | Degradation product |
| B3 | 0.77 | 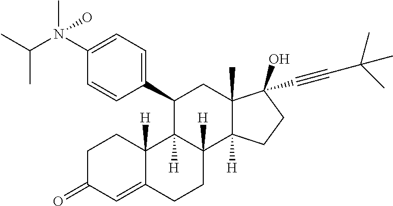 | Degradation product |
| B4 | 0.78/0.79 | 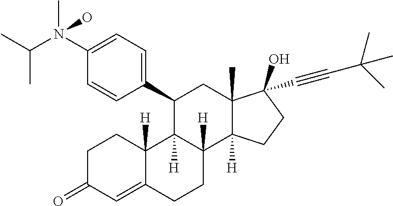 | Degradation product |
| B5 | 0.49 | 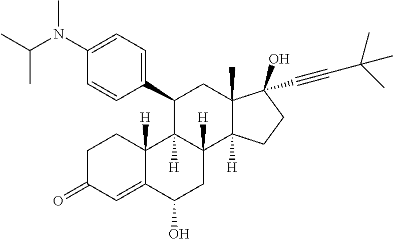 | Degradation product |
| B6 | 0.67 | 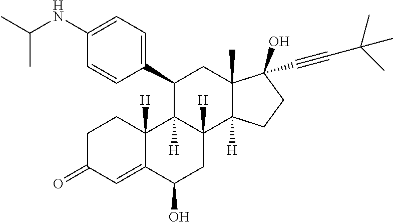 | Degradation product |

### Methods of Treatment

Disclosed herein is a method of treating non-small cell lung cancer, triple negative breast cancer, ovarian cancer, melanoma, pancreatic cancer, prostate cancer, castration resistant prostate cancer, renal cancer, melanoma, hepatocellular carcinoma, or bladder cancer, in a subject in need thereof; the method comprising administering a formulation comprising a compound of Formula (I), or a pharmaceutically acceptable salt thereof (e.g., Compound 1, or a pharmaceutically acceptable salt thereof).

Disclosed herein is a method of treating non-small cell lung cancer, triple negative breast cancer, ovarian cancer, castration resistant prostate cancer, renal cancer, melanoma, hepatocellular carcinoma, or bladder cancer, in a subject in need thereof; the method comprising administering a formulation comprising a compound of Formula (I), or a pharmaceutically acceptable salt thereof (e.g., Compound 1, or a pharmaceutically acceptable salt thereof).

In some embodiments, the compound of Formula (I), or a pharmaceutically acceptable salt thereof (e.g., Compound 1, or a pharmaceutically acceptable salt thereof) is used in combination with a second therapeutic agent (e.g., an anti-cancer agent) for treating cancer. In some embodiments, the combination of the compound of Formula (I), or a pharmaceutically acceptable salt thereof (e.g., Compound 1, or a pharmaceutically acceptable salt thereof) with the second therapeutic agent (e.g., an anti-cancer agent) provides a more effective initial therapy for treating cancer compared to the second therapeutic agent (e.g., an anti-cancer agent) administered alone.

In some embodiments, the cancer disclosed herein is chemoresistant cancer, radio resistant cancer, or refractory cancer. In some embodiments, the cancer is relapsed cancer, persistent cancer, or recurrent cancer. Another embodiment provided herein describes a method of reducing incidences of cancer recurrence. Also provided here in some embodiments, is a method for treating a chemo-resistant cancer.

### Prostate Cancer

Prostate cancer is the second most common cause of cancer death in men in the United States, and approximately one in every six American men will be diagnosed with the disease during his lifetime. Treatment aimed at eradicating the tumor is unsuccessful in 30% of men.

One embodiment provides a method of treating prostate cancer in a subject in need thereof, comprising administering to the subject a formulation comprising a compound of Formula (I), or a pharmaceutically acceptable salt thereof (e.g., Compound 1, or a pharmaceutically acceptable salt thereof).

In some embodiments, the prostate cancer is chemoresistant cancer, radio resistant cancer, antiandrogen resistant, or refractory cancer. In some embodiments, the prostate cancer is relapsed cancer, persistent cancer, or recurrent cancer.

In some embodiments, the prostate cancer is acinar adenocarcinoma, atrophic carcinoma, foamy carcinoma, colloid carcinoma, or signet ring carcinoma. In some embodiments, the prostate cancer is ductal adenocarcinoma, transitional cell cancer, urothelial cancer, squamous cell cancer, carcinoid cancer, small cell cancer, sarcoma cancer, or sarcomatoid cancer. In some embodiments, the prostate cancer is metastatic castration-resistant prostate cancer, doubly-resistant prostate cancer, castration-resistant prostate cancer, hormone-resistant prostate cancer, androgen-independent, or androgen-refractory cancer.

In some instances, antiandrogens are useful for the treatment of prostate cancer during its early stages. In some instances, prostate cancer cells depend on androgen receptor (AR) for their proliferation and survival. Some prostate cancer patients are physically castrated or chemically castrated by treatment with agents that block production of testosterone (e.g. GnRH agonists), alone or in combination with antiandrogens, which antagonize effects of any residual testosterone.

In some instances, prostate cancer advances to a hormone-refractory state in which the disease progresses despite continued androgen ablation or antiandrogen therapy. The hormone-refractory state to which most patients eventually progresses in the presence of continued androgen ablation or anti-androgen therapy is known as “castration resistant” prostate cancer (CRPC). CRPC is associated with an overexpression of AR. AR is expressed in most prostate cancer cells and overexpression of AR is necessary and sufficient for androgen-independent growth of prostate cancer cells. Failure in hormonal therapy, resulting from development of androgen-independent growth, is an obstacle for successful management of advanced prostate cancer.

While a small minority of CRPC does bypass the requirement for AR signaling, the vast majority of CRPC, though frequently termed “androgen independent prostate cancer” or “hormone refractory prostate cancer,” retains its lineage dependence on AR signaling.

Recently approved therapies that target androgen receptor (AR) signaling such as abiraterone and enzalutamide have been utilized for treating CRPC. Despite these successes, sustained response with these agents is limited by acquired resistance which typically develops within 6-12 months. Doubly resistant prostate cancer is characterized in that tumor cells have become castration resistant and overexpress AR, a hallmark of CRPC. However, cells remain resistant when treated with second generation antiandrogens. Doubly resistant prostate cancer cells are characterized by a lack of effectiveness of second generation antiandrogens in inhibiting tumor growth.

As discussed above, resistant prostate cancer (e.g., doubly resistant and castration resistant prostate cancers) occurs when cancer cells overexpress androgen receptors (AR). AR target gene expression is inhibited when the cells are treated with a second generation antiandrogen. In some instances, increased signaling through the glucocorticoid receptor (GR) compensates for inhibition of androgen receptor signaling in resistant prostate cancer. Double resistant prostate cancer develops when expression of a subset of those AR target genes is restored. In some instances, GR activation is responsible for this target gene activation. In some embodiments, GR transcription is activated in patients susceptible to or suffering from resistant prostate cancer (e.g., doubly resistant and castration resistant prostate cancers). In some instances, GR upregulation in cancer cells confers resistance to antiandrogens.

Some embodiments provided herein describe the use of the GR inhibitors for treating prostate cancer in a subject in need thereof, including doubly resistant prostate cancer and castration resistant prostate cancer. In some embodiments, the subject in need has elevated tumor GR expression. In some embodiments, the GR inhibitor is also an AR signaling inhibitor or antiandrogen.

In some embodiments, the formulation comprising a compound of Formula (I), or a pharmaceutically acceptable salt thereof (e.g., Compound 1, or a pharmaceutically acceptable salt thereof) is used in combination with an anti-cancer agent or an AR signaling inhibitor or antiandrogen.

In some embodiments, the second or additional agent is an AR signaling inhibitor or antiandrogen. In certain embodiments, the AR signaling inhibitor is an AR antagonist. In some embodiments, the second or additional therapeutic agent is selected from finasteride, dutasteride, alfatradiol, cyproterone acetate, spironolactone, danazol, gestrinone, ketoconazole, abiraterone acetate, enzalutamide, apalutamide, darolutamide, danazol, gestrinone, danazol, simvastatin, aminoglutethimide, atorvastatin, simvastatin, progesterone, cyproterone acetate, medroxyprogesterone acetate, megestrol acetate, chlormadinone acetate, spironolactone, drospirenone, estradiol, ethinyl estradiol, diethylstilbestrol, conjugated equine estrogens, buserelin, deslorelin, gonadorelin, goserelin, histrelin, leuprorelin, nafarelin, triptorelin, abarelix, cetrorelix, degarelix, ganirelix, or any combinations or any salts thereof. In some embodiments, the second or additional therapeutic agent is selected from flutamide, nilutamide, bicalutamide, enzalutamide, apalutamide, darolutamide, cyproterone acetate, megestrol acetate, chlormadinone acetate, spironolactone, canrenone, drospirenone, ketoconazole, topilutamide, cimetidine, or any combinations or any salts thereof. In some embodiments, the AR signaling inhibitor is 3,3′-diindolylmethane (DIM), abiraterone acetate, apalutamide, darolutamide, bexlosteride, bicalutamide, dutasteride, epristeride, enzalutamide, finasteride, flutamide, izonsteride, ketoconazole, N-butylbenzene-sulfonamide, nilutamide, megestrol, steroidal antiandrogens, turosteride, or any combinations thereof. In some embodiments, the AR signaling inhibitor is flutamide, nilutamide, bicalutamide, or megestrol. In some embodiments, the AR signaling inhibitor is apalutamide. In other embodiments, the AR signaling inhibitor is enzalutamide.

In some embodiments, the anti-cancer agent is mitoxantrone, estramustine, etoposide, vinblastine, carboplatin, vinorelbine, paclitaxel, daunomycin, darubicin, epirubicin, docetaxel, cabazitaxel, or doxorubicin. In some embodiments, the anti-cancer agent is paclitaxel, daunomycin, darubicin, epirubicin, docetaxel, cabazitaxel, or doxorubicin. In certain embodiments, the anti-cancer agent is docetaxel.

### Breast Cancer

Breast cancer is the second leading cause of cancer among women in the United States. Triple-negative breast cancers are among the most aggressive and difficult to treat of all the breast cancer types. Triple-negative breast cancer is a form of the disease in which the three receptors that fuel most breast cancer growth—estrogen, progesterone and the HER-2—are not present. Because the tumor cells lack these receptors, treatments that target estrogen, progesterone and HER-2 are ineffective. Approximately 40,000 women are diagnosed with triple-negative breast cancer each year. It is estimated that more than half of these women's tumor cells express significant amounts of GR.

In some instances, GR expression is associated with a poor prognosis in estrogen receptor (ER)-negative early stage breast cancer. In some instances, GR activation in triple-negative breast cancer cells initiates an anti-apoptotic gene expression profile that is associated with inhibiting chemotherapy-induced tumor cell death. GR activity in these cancer cells correlate with chemotherapy resistance and increased recurrence of cancer.

Provided herein in some embodiments are methods of treating breast cancer, the method comprising administering to a subject in need thereof a formulation comprising a compound of Formula (I), or a pharmaceutically acceptable salt thereof (e.g., Compound 1, or a pharmaceutically acceptable salt thereof). In some embodiments, a GR inhibitor described herein is used in combination with a second therapeutic agent (e.g., a chemotherapeutic agent) for treating breast cancer. In some embodiments, the combination of the GR inhibitor with the second therapeutic agent (e.g., a chemotherapeutic agent) provides a more effective initial therapy for treating breast cancer compared to the second therapeutic agent (e.g., a chemotherapeutic agent) administered alone.

In some embodiments, the breast cancer is chemoresistant cancer, radio resistant cancer, or refractory cancer. In some embodiments, the breast cancer is relapsed cancer, persistent cancer, or recurrent cancer. Breast cancers may include, but are not limited to, ductal carcinoma, invasive ductal carcinoma, tubular carcinoma of the breast, medullary carcinoma of the breast, mecinous carcinoma of the breast, papillary carcinoma of the breast, cribriform carcinoma of the breast, invasive lobular carcinoma, inflammatory breast cancer, lobular carcinoma in situ, male breast cancer, Paget disease of the nipple, phyllodes tumor of the breast, recurrent and metastatic breast cancer, triple-negative breast cancer, or combinations thereof.

In some embodiments, the breast cancer is recurrent and metastatic breast cancer, triple-negative breast cancer, or combinations thereof. In some embodiments, the breast cancer is chemoresistant triple-negative breast cancer or estrogen receptor (ER) negative breast cancer. In some embodiments, the breast cancer is chemoresistant triple-negative breast cancer. In some embodiments, the breast cancer is estrogen receptor (ER) negative breast cancer. In some embodiments, the breast cancer is GR+ triple-negative breast cancer. In some embodiments, the breast cancer is GR+ estrogen receptor (ER) negative breast cancer.

Some embodiments provided herein describe the use of a compound of Formula (I), or a pharmaceutically acceptable salt thereof (e.g., Compound 1, or a pharmaceutically acceptable salt thereof) for treating breast cancer in a patient, including triple negative breast cancer or ER negative breast cancer. In some embodiments, GR inhibitors inhibit the anti-apoptotic signaling pathways of GR and increase the cytotoxic efficiency of secondary chemotherapeutic agents. In some embodiments, the GR inhibitors described herein enhance the efficacy of chemotherapy in breast cancer patients, such as triple negative breast cancer patients. In some embodiments, the breast cancer patient has elevated tumor GR expression.

In some embodiments, the formulation comprising a GR inhibitor described herein is used in combination with a second therapeutic agent, such as chemotherapy or immunotherapy. In some embodiments, a GR inhibitor described herein is used in combination with one or more additional therapeutic agents. In some embodiments, the second or additional chemotherapeutic agent is cisplatin, carboplatin, cyclophosphamide, capecitabine, gemcitabine, paclitaxel, nab-paclitaxel, altretamine, docetaxel, epirubicin, melphalan, methotrexate, mitoxantrone, ixabepilone, ifosfamide, irinotecan, eribulin, etoposide, doxorubicin, liposomal doxorubicin, camptothecin, pemetrexed, topotecan, vinorelbine, vinblastine, daunorubicin, 5-fluorouracil, mitomycin, thiotepa, vincristine, everolimus, veliparib, glembatumumab vedotin, pertuzumab, trastuzumab, or any combinations or any salts thereof. In some embodiments, the second or additional therapeutic agent is an anti-PD-L1 agent. In certain embodiments, the anti-PD-L1 agent is MPDL3280A or avelumab. In some embodiments, the second or additional therapeutic agent is an anti-PD1 agent. In certain embodiments, the anti-PD1 agent is nivolumab or permbrolizumab. In some embodiments, the second or additional therapeutic agent is an anti an anti-CTLA-4 agent. In some embodiments, the second or additional therapeutic agent is a CAR-T cell therapy. In some embodiments, the second or additional therapeutic agent is an IDO-1 inhibitor. In some embodiments, the second or additional therapeutic agent is a cancer vaccine.

Some embodiments provided herein describe methods of treating estrogen positive breast cancer. In some instances, estrogen positive breast cancer patients become resistant to estrogen receptor modulators. In some embodiments, the GR inhibitors described herein enhance the efficacy of estrogen receptor modulators in estrogen positive breast cancer patients. In some embodiments, the breast cancer patient has elevated tumor GR expression. In some embodiments, a GR inhibitor described herein is used in combination with an estrogen receptor modulator. In some embodiments, the estrogen receptor modulator is tamoxifen, raloxifene, toremifene, tibolone, fulvestrant, lasofoxifene, clomifene, ormeloxifene, or ospemifene. In some embodiments, the estrogen receptor modulator is tamoxifen, raloxifene, toremifene, tibolone, or fulvestrant. In some embodiments, the estrogen receptor modulator is tamoxifen, raloxifene, or toremifene. In certain embodiments, the estrogen receptor modulator is tamoxifen.

### Ovarian Cancer

Ovarian cancer is the leading cause of death from gynecologic malignancies. Some ovarian cancers (e.g., high grade serous ovarian cancer) are initially sensitive to platinum-based therapy, but relapse rates remain high.

One embodiment provides a method of treating ovarian cancer in a patient in need thereof, comprising administering to the patient a formulation comprising a compound of Formula (I), or a pharmaceutically acceptable salt thereof (e.g., Compound 1, or a pharmaceutically acceptable salt thereof). In some embodiments, the patient has elevated tumor GR expression. In some embodiments, a formulation comprising a compound of Formula (I), or a pharmaceutically acceptable salt thereof (e.g., Compound 1, or a pharmaceutically acceptable salt thereof) is used in combination with a second therapeutic agent (e.g., a chemotherapeutic agent) for treating ovarian cancer. In some embodiments, the combination of the GR inhibitor with the second therapeutic agent (e.g., a chemotherapeutic agent) provides a more effective initial therapy for treating ovarian cancer compared to the second therapeutic agent (e.g., a chemotherapeutic agent) administered alone.

In some instances, GR activation increases resistance to chemotherapy in ovarian cancer (e.g., high-grade serous ovarian cancer). In some instances, GR activation significantly inhibits chemotherapy induced apoptosis in ovarian cancer cells. Provided herein in some embodiments are methods of treating ovarian cancer in a subject, the method comprising treating the subject with a GR inhibitor (e.g., GR antagonist) to improve sensitivity to chemotherapy. In some embodiments, the ovarian cancer has become resistant to chemotherapy. In some embodiments, the ovarian cancer cells are resistant to cisplatin, paclitaxel, carboplatin, gemcitabine, alone or in combination. In some embodiments, the GR inhibitor or antagonist reverses the cell survival effect.

Ovarian cancers may include, but are not limited to, epithelial ovarian cancers, such as serous epithelial ovarian cancer, endometrioid epithelial ovarian cancer, clear cell epithelial ovarian cancer, mucinous epithelial ovarian cancer, undifferentiated or unclassifiable epithelial ovarian cancer, refractory ovarian cancer, sex cord-stromal tumors, Sertoli and Sertoli-Leydig cell tumors, germ cell tumors, such as dysgerminoma and nondysgerminomatous tumors, Brenner tumors, primary peritoneal carcinoma, fallopian tube cancer, or combinations thereof.

In some embodiments, the formulation comprising compound of Formula (I), or a pharmaceutically acceptable salt thereof (e.g., Compound 1, or a pharmaceutically acceptable salt thereof) is used in combination with at least a second therapeutic agent, such as chemotherapy or immunotherapy. In some embodiments, the second or additional chemotherapeutic agent is cisplatin, carboplatin, cyclophosphamide, capecitabine, gemcitabine, paclitaxel, nab-paclitaxel, altretamine, docetaxel, epirubicin, melphalan, methotrexate, mitoxantrone, ixabepilone, ifosfamide, irinotecan, eribulin, etoposide, doxorubicin, liposomal doxorubicin, camptothecin, pemetrexed, topotecan, vinorelbine, vinblastine, daunorubicin, 5-fluorouracil, mitomycin, thiotepa, vincristine, everolimus, veliparib, glembatumumab vedotin, pertuzumab, trastuzumab, or any combinations or any salts thereof. In some embodiments, the second or additional chemotherapeutic agent is gemcitabine. In some embodiments, the second or additional chemotherapeutic agent is carboplatin. In some embodiments, the second or additional chemotherapeutic agent is cisplatin. In some embodiments, the second or additional agent is paclitaxel. In some embodiments, the GR inhibitor is used in combination with gemcitabine and carboplatin. In some embodiments, the GR inhibitor is used in combination with carboplatin and cisplatin. In some embodiments, the second or additional therapeutic agent is an anti-PD-L1 agent. In certain embodiments, the anti-PD-L1 agent is MPDL3280A or avelumab. In some embodiments, the second or additional therapeutic agent is an anti-PD1 agent. In certain embodiments, the anti-PD1 agent is nivolumab or permbrolizumab. In some embodiments, the second or additional therapeutic agent is an anti an anti-CTLA-4 agent. In some embodiments, the second or additional therapeutic agent is a CAR-T cell therapy. In some embodiments, the second or additional therapeutic agent is an IDO-1 inhibitor. In some embodiments, the second or additional therapeutic agent is a cancer vaccine.

### Non-Small Cell Lung Cancer

One embodiment provides a method of treating non-small cell lung cancer (NSCLC) in a patient in need thereof, comprising administering to the patient a formulation provided herein. In some embodiments, the patient has elevated tumor GR expression. In some embodiments, a GR inhibitor described herein is used in combination with a second therapeutic agent (e.g., a chemotherapeutic agent) for treating NSCLC. In some embodiments, the combination of the GR inhibitor with the second therapeutic agent (e.g., a chemotherapeutic agent) provides a more effective initial therapy for treating NSCLC compared to the second therapeutic agent (e.g., a chemotherapeutic agent) administered alone.

In some embodiments, the formulation comprising a compound of Formula (I), or a pharmaceutically acceptable salt thereof (e.g., Compound 1, or a pharmaceutically acceptable salt thereof) is used in combination with at least a second therapeutic agent, such as a chemotherapeutic agent or immunotherapy. In some embodiments, the second or additional chemotherapeutic agent is cisplatin, carboplatin, cyclophosphamide, capecitabine, gemcitabine, paclitaxel, nab-paclitaxel, altretamine, docetaxel, epirubicin, melphalan, methotrexate, mitoxantrone, ixabepilone, ifosfamide, irinotecan, eribulin, etoposide, doxorubicin, liposomal doxorubicin, camptothecin, pemetrexed, topotecan, vinorelbine, vinblastine, daunorubicin, 5-fluorouracil, mitomycin, thiotepa, vincristine, everolimus, veliparib, glembatumumab vedotin, pertuzumab, trastuzumab, or any combinations or any salts thereof. In some embodiments, the second or additional chemotherapeutic agent is gemcitabine. In some embodiments, the second or additional chemotherapeutic agent is carboplatin. In some embodiments, the second or additional chemotherapeutic agent is cisplatin. In some embodiments, the second or additional agent is paclitaxel. In some embodiments, the GR inhibitor is used in combination with gemcitabine and carboplatin. In some embodiments, the GR inhibitor is used in combination with carboplatin and cisplatin. In some embodiments, the second or additional therapeutic agent is an anti-PD-L1 agent. In certain embodiments, the anti-PD-L1 agent is MPDL3280A or avelumab. In some embodiments, the second or additional therapeutic agent is an anti-PD1 agent. In certain embodiments, the anti-PD1 agent is nivolumab or permbrolizumab. In some embodiments, the second or additional therapeutic agent is an anti an anti-CTLA-4 agent. In some embodiments, the second or additional therapeutic agent is a CAR-T cell therapy. In some embodiments, the second or additional therapeutic agent is an IDO-1 inhibitor. In some embodiments, the second or additional therapeutic agent is a cancer vaccine.

### Dosing

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for a compound of Formula (I), or a pharmaceutically acceptable salt thereof (e.g., Compound 1, or a pharmaceutically acceptable salt thereof) can be determined via established animal and human experimental protocols as well as in the examples described herein. For example, toxicity and therapeutic efficacy of a compound of Formula (I), or a pharmaceutically acceptable salt thereof (e.g., Compound 1, or a pharmaceutically acceptable salt thereof) can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given compound of Formula (I), or a pharmaceutically acceptable salt thereof (e.g., Compound 1, or a pharmaceutically acceptable salt thereof) formulation that corresponds to such an amount varies depending upon factors such as the particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, the formulations described herein provide a dose of the compound of Formula (I) (e.g., Compound 1) from about 10 mg to 1000 mg, from about 10 mg to about 200 mg, from about 100 to about 500, or from about 200 mg to about 800 mg. In some embodiments, the formulations described herein provide a dose of the compound of Formula (I) (e.g., Compound 1) of about 200 mg, about 300 mg, about 400 mg, about 500 mg, about 600 mg, about 700 mg, or about 800 mg.

### Administration

Administration of a formulation (or composition) described is at a dosage described herein or at other dose levels and compositions determined and contemplated by a medical practitioner. In certain embodiments, the formulations and compositions described herein are administered for prophylactic and/or therapeutic treatments. In certain therapeutic applications, the compositions are administered to a patient already suffering from a disease in an amount sufficient to cure the disease or at least partially arrest or ameliorate the symptoms. Amounts effective for this use depend on the age of the patient, severity of the disease, previous therapy, the patient's health status, weight, and response to the compositions, and the judgment of the treating physician. Therapeutically effective amounts are optionally determined by methods including, but not limited to, a dose escalation clinical trial.

In prophylactic applications, the compositions described herein are administered to a patient susceptible to or otherwise at risk of a particular disease, e.g., cancer. Such an amount is defined to be a “prophylactically effective amount or dose.” In this use, the precise amounts also depend on the patient's age, state of health, weight, and the like. When used in a patient, effective amounts for this use will depend on the risk or susceptibility of developing the particular disease, previous therapy, the patient's health status and response to the compositions, and the judgment of the treating physician.

In certain embodiments wherein the patient's condition does not improve, upon the doctor's discretion the administration of an composition described herein are administered chronically, that is, for an extended period of time, including throughout the duration of the patient's life in order to ameliorate or otherwise control or limit the symptoms of the patient's disease. In other embodiments, administration of a composition continues until complete or partial response of a disease.

In some embodiments, the formulations described herein are administered once a day. In some embodiments, the formulations described herein are administered twice a day. In some embodiments, the formulations described herein are administered three times a day. In some embodiments, the formulations described herein are administered every other a day.

In some embodiments, compositions described herein are administered chronically. For example, in some embodiments, a composition is administered as a continuous dose, i.e., administered daily to a subject. In some other embodiments, compositions described herein are administered intermittently (e.g. drug holiday that includes a period of time in which the composition is not administered or is administered in a reduced amount).

In some embodiments, a composition is administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, a composition is administered orally to a subject who is in a fasted state for at least 8 hours. In other embodiments, a composition is administered to a subject who is in a fasted state for at least 10 hours. In yet other embodiments, a liquid composition is administered to a subject who is in a fasted state for at least 12 hours. In other embodiments, a composition is administered to a subject who has fasted overnight.

In other embodiments a composition is administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, a composition is administered to a subject in a fed state 30 minutes post-meal. In other instances, a composition is administered to a subject in a fed state 1 hour post-meal. In yet further embodiments, a composition is administered to a subject with food.

In some instances, the methods described herein further comprise administering the compositions and formulations comprising a compound of Formula (I) (e.g., Compound 1), or pharmaceutically acceptable salt thereof, in combination with a second therapeutic agent to the subject or patient in need thereof in multiple cycles repeated on a regular schedule with periods of rest in between each cycle. For example, in some instances, treatment is given for one week followed by three weeks of rest is one treatment cycle.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is 56 days. In some embodiments, a treatment cycle lasts one, two, three, or four weeks. In some embodiments, a treatment cycle lasts three weeks. In some embodiments, a treatment cycle lasts four weeks. The number of treatment doses scheduled within each cycle also varies depending on the drugs being given.

In some instances, the method for the administration of multiple compounds comprises administering compounds within 48 hours or less of each other. In some embodiments administration occurs within 24 hours, 12 hours, 6 hours, 3 hours, 1 hour, or 15 minutes. In some instances, the compounds are administered simultaneously. One example of simultaneous administration is the injection of one compound immediately before, after, or during the oral administration of the second compound, immediately referring to a time less than about 5 minutes.

In some instances, the method for the administration of multiple compounds occurs in a sequential order, wherein the compositions and formulations comprising a compound of Formula (I) (e.g., Compound 1), or pharmaceutically acceptable salt thereof, is administered before the second therapeutic agent. In another instance, the second therapeutic agent is administered before the compositions and formulations comprising a compound of Formula (I) (e.g., Compound 1), or pharmaceutically acceptable salt thereof.

In some instances, the method for administering the compositions and formulations comprising a compound of Formula (I) (e.g., Compound 1), or pharmaceutically acceptable salt thereof is oral and the method for administering the second therapeutic agent is by injection. In other instances, the method for administering the compositions and formulations comprising a compound of Formula (I) (e.g., Compound 1), or pharmaceutically acceptable salt thereof, is by injection and the method for administering the second therapeutic agent is by injection.

In certain embodiments, a compound of Formula (I), or a pharmaceutically acceptable salt thereof, and a second therapeutic agent is cyclically administered to a patient. As discussed above, cycling therapy involves the administration of an active agent or a combination of active agents for a period of time, optionally followed by a rest for a period of time (e.g., a “drug holiday”), and repeating this sequential administration. In some embodiments, cycling therapy reduces the development of resistance to one or more of the therapies, avoid or reduce the side effects of one of the therapies, and/or improves the efficacy of the treatment.

In some embodiments, the compound of Formula (I), or a pharmaceutically acceptable salt thereof, is administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the compound of Formula (I), or a pharmaceutically acceptable salt thereof, is administered daily. In some embodiments, the compound of Formula (I), or a pharmaceutically acceptable salt thereof, is administered daily for the first 7 days of a 3 week cycle. In some embodiments, the compound of Formula (I), or a pharmaceutically acceptable salt thereof, is administered on day 1, day 8, and day 15 of a 3 week cycle. In some embodiments, the compound of Formula (I), or a pharmaceutically acceptable salt thereof, is administered on day 1 of a 3 week cycle.

In some embodiments, the compound of Formula (I), or a pharmaceutically acceptable salt thereof, is administered on one day of a 3 week cycle. In some embodiments, the compound of Formula (I), or a pharmaceutically acceptable salt thereof, is administered on two days of a 3 week cycle. In some embodiments, the compound of Formula (I), or a pharmaceutically acceptable salt thereof, is administered on three days of a 3 week cycle. In some embodiments, the compound of Formula (I), or a pharmaceutically acceptable salt thereof, is administered on four days of a 3 week cycle. In some embodiments, the compound of Formula (I), or a pharmaceutically acceptable salt thereof, is administered on five days of a 3 week cycle. In some embodiments, the compound of Formula (I), or a pharmaceutically acceptable salt thereof, is administered on six days of a 3 week cycle. In some embodiments, the compound of Formula (I), or a pharmaceutically acceptable salt thereof, is administered on seven days of a 3 week cycle. In some embodiments, the compound of Formula (I), or a pharmaceutically acceptable salt thereof, is administered on eight days of a 3 week cycle. In some embodiments, the compound of Formula (I), or a pharmaceutically acceptable salt thereof, is administered on nine days of a 3 week cycle.

In some embodiments, the compound of Formula (I), or a pharmaceutically acceptable salt thereof, is administered on one day of each week per 3 week cycle. In some embodiments, the compound of Formula (I), or a pharmaceutically acceptable salt thereof, is administered for 2 days of each week per 3 week cycle. In some embodiments, the compound of Formula (I), or a pharmaceutically acceptable salt thereof, is administered for 3 days of each week per 3 week cycle. In some embodiments, the compound of Formula (I), or a pharmaceutically acceptable salt thereof, is administered for 4 days of each week per 3 week cycle. In some embodiments, the compound of Formula (I), or a pharmaceutically acceptable salt thereof, is administered for 5 days of each week per 3 week cycle. In some embodiments, the compound of Formula (I), or a pharmaceutically acceptable salt thereof, is administered for 6 days of each week per 3 week cycle. In some embodiments, the compound of Formula (I), or a pharmaceutically acceptable salt thereof, is administered for 7 days of each week per 3 week cycle (i.e., every day of the 3 week cycle).

In some embodiments, the second therapeutic agent is administered daily, every other day, every other day 3 times a week, every 3 days, every 4 days, every 5 days, every 6 days, weekly, every 2 weeks, every 3 weeks, every 4 weeks, every 5 weeks, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the second therapeutic agent is administered daily. In some embodiments, the second therapeutic agent is administered daily for the first 7 days of a 3 week cycle. In some embodiments, the second therapeutic agent is administered on day 1, day 8, and day 15 of a 3 week cycle. In some embodiments, the second therapeutic agent is administered on day 1 of a 3 week cycle (i.e., day 1 of each 21 day cycle). In some embodiments, the second therapeutic agent is administered on day 1, day 8, and day 15 of a 3 week cycle (21-day cycle) and thereafter on day 1 of each 21-day cycle.

In some embodiments, the second therapeutic agent is administered on one day of a 3 week cycle. In some embodiments, the second therapeutic agent is administered on two days of a 3 week cycle. In some embodiments, the second therapeutic agent is administered on three days of a 3 week cycle. In some embodiments, the second therapeutic agent is administered on four days of a 3 week cycle. In some embodiments, the second therapeutic agent is administered on five days of a 3 week cycle. In some embodiments, the second therapeutic agent is administered on six days of a 3 week cycle. In some embodiments, the second therapeutic agent is administered on seven days of a 3 week cycle. In some embodiments, the second therapeutic agent is administered on eight days of a 3 week cycle. In some embodiments, the second therapeutic agent is administered on nine days of a 3 week cycle.

In some embodiments, the second therapeutic agent is administered on one day of each week per 3 week cycle. In some embodiments, the second therapeutic agent is administered for 2 days of each week per 3 week cycle. In some embodiments, the second therapeutic agent is administered for 3 days of each week per 3 week cycle. In some embodiments, the second therapeutic agent is administered for 4 days of each week per 3 week cycle. In some embodiments, the second therapeutic agent is administered for 5 days of each week per 3 week cycle. In some embodiments, the second therapeutic agent is administered for 6 days of each week per 3 week cycle. In some embodiments, the second therapeutic agent is administered for 7 days of each week per 3 week cycle (i.e., every day of the 3 week cycle).

In some instances, the compound of Formula (I), or a pharmaceutically acceptable salt thereof, or the second therapeutic agent is optionally given continuously; alternatively, the dose of drug being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 8 days, 9 days, 10 days, 12 days, 14 days, 15 days, 20 days, 21 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In some instances, the method for multiple cycle chemotherapy comprises the administration of a second cycle within about 60 days or about 3 months. In some instances, the method for multiple cycle chemotherapy comprises the administration of a second cycle within 50 days. In another instance, the second cycle is administered within 45, 40, 35, 30, 25, 21, 20, 15, 14, 10, 9, 8, 7, 6, 5, 4, 3, 2 or 1 day(s) of the first cycle. In some embodiments, the administration of any additional cycles is within 50 days of the previous cycle. In some embodiments, the administration of any additional cycles is within 10 days of the previous cycle. In some embodiments, the administration of any additional cycles is within 9 days of the previous cycle. In some embodiments, the administration of any additional cycles is within 8 days of the previous cycle. In some embodiments, the administration of any additional cycles is within 7 days of the previous cycle. In some embodiments, the administration of any additional cycles is within 6 days of the previous cycle. In some embodiments, the administration of any additional cycles is within 5 days of the previous cycle. In some embodiments, the administration of any additional cycles is within 4 days of the previous cycle. In some embodiments, the administration of any additional cycles is within 3 days of the previous cycle. In some embodiments, the administration of any additional cycles is within 2 days of the previous cycle. In some embodiments, the administration of any additional cycles is within 1 day of the previous cycle. In another embodiment, the additional cycle is administered within 45, 40, 35, 30, 25, 21, 20, 15, 14, 10, 9, 8, 7, 6, 5, 4, 3, 2, or 1 days of the previous cycle.

In certain embodiments, the second therapeutic agent used in combination with a compound of Formula (I), or a pharmaceutically acceptable salt thereof, is paclitaxel. In certain embodiments, the methods described herein further comprise administering paclitaxel as an intravenous infusion in a 3 week cycle (i.e., in 21-day cycles). In certain embodiments, paclitaxel is administered as an intravenous infusion for multiple 3 week cycles (21-day cycles). In some embodiments, paclitaxel is administered on day 1 of a 3 week cycle. In certain embodiments, paclitaxel is administered on day 1, day 8, and day 15 of a 3 week cycle. In some embodiments, paclitaxel is administered once a week per three week cycle.

In certain embodiments, paclitaxel is administered as an intravenous infusion at a dose of 135 mg/m² intravenously over 3 hours every 3 weeks. In certain embodiments, paclitaxel is administered as an intravenous infusion at a dose of 175 mg/m² intravenously over 3 hours every 3 weeks.

In some embodiments, a compound of Formula (I) (e.g., Compound 1), or a pharmaceutically acceptable salt thereof, is administered for the first week of a three week cycle (i.e., days 1-7 of a 21-day cycle) and paclitaxel is administered on day 1 of the three week cycle. In some embodiments, a compound of Formula (I) (e.g., Compound 1), or a pharmaceutically acceptable salt thereof, is administered for the first week of a three week cycle (i.e., days 1-7 of a 21-day cycle) and paclitaxel is administered on three days of the three week cycle. In some embodiments, a compound of Formula (I) (e.g., Compound 1), or a pharmaceutically acceptable salt thereof, is administered for the first week of a three week cycle (i.e., days 1-7 of a 21-day cycle) and paclitaxel is administered on days 1, 8, and 15 of the three week cycle.

In some embodiments, a compound of Formula (I) (e.g., Compound 1), or a pharmaceutically acceptable salt thereof, is administered daily for a three week cycle (i.e., days 1-21 of a 21-day cycle) and paclitaxel is administered on day 1 of the three week cycle. In some embodiments, a compound of Formula (I) (e.g., Compound 1), or a pharmaceutically acceptable salt thereof, is administered daily for a three week cycle (i.e., days 1-21 of a 21-day cycle) and paclitaxel is administered on three days of the three week cycle. In some embodiments, a compound of Formula (I) (e.g., Compound 1), or a pharmaceutically acceptable salt thereof, is administered daily for a three week cycle (i.e., days 1-21 of a 21-day cycle) and paclitaxel is administered on days 1, 8, and 15 of the three week cycle.

In certain embodiments, the second therapeutic agent used in combination with a compound of Formula (I), or a pharmaceutically acceptable salt, is Abraxane. In certain embodiments, the methods described herein further comprise administering Abraxane as an intravenous infusion in a 3 week cycle (i.e., in 21-day cycles). In certain embodiments, Abraxane is administered as an intravenous infusion for multiple 3 week cycles (21-day cycles). In some embodiments, Abraxane is administered on day 1 of a 3 week cycle. In certain embodiments, Abraxane is administered on day 1, day 8, and day 15 of a 3 week cycle. In some embodiments, Abraxane is administered once a week per three week cycle.

In certain embodiments, Abraxane is administered as an intravenous infusion at a dose of 80 mg/m² IV infusion for 30 minute/week. In certain embodiments, Abraxane is administered as an intravenous infusion at a dose of 100 mg/m² IV infusion for 30 minute/week.

In some embodiments, a compound of Formula (I) (e.g., Compound 1), or a pharmaceutically acceptable salt thereof, is administered for the first week of a three week cycle (i.e., days 1-7 of a 21-day cycle) and Abraxane is administered on day 1 of the three week cycle. In some embodiments, a compound of Formula (I) (e.g., Compound 1), or a pharmaceutically acceptable salt thereof, is administered for the first week of a three week cycle (i.e., days 1-7 of a 21-day cycle) and Abraxane is administered on three days of the three week cycle. In some embodiments, a compound of Formula (I) (e.g., Compound 1), or a pharmaceutically acceptable salt thereof, is administered for the first week of a three week cycle (i.e., days 1-7 of a 21-day cycle) and Abraxane is administered on days 1, 8, and 15 of the three week cycle.

In some embodiments, a compound of Formula (I) (e.g., Compound 1), or a pharmaceutically acceptable salt thereof, is administered daily for a three week cycle (i.e., days 1-21 of a 21-day cycle) and Abraxane is administered on day 1 of the three week cycle. In some embodiments, a compound of Formula (I) (e.g., Compound 1), or a pharmaceutically acceptable salt thereof, is administered daily for a three week cycle (i.e., days 1-21 of a 21-day cycle) and Abraxane is administered on three days of the three week cycle. In some embodiments, a compound of Formula (I) (e.g., Compound 1), or a pharmaceutically acceptable salt thereof, is administered daily for a three week cycle (i.e., days 1-21 of a 21-day cycle) and Abraxane is administered on days 1, 8, and 15 of the three week cycle.

In certain embodiments, the second therapeutic agent used in combination with a compound of Formula (I), or a pharmaceutically acceptable salt thereof, is Keytruda. In certain embodiments, the methods described herein further comprise administering Keytruda as an intravenous infusion in a 3 week cycle (i.e., in 21-day cycles). In certain embodiments, Keytruda is administered as an intravenous infusion for multiple 3 week cycles (21-day cycles). In some embodiments, Keytruda is administered on day 1 of a 3 week cycle. In certain embodiments, Keytruda is administered on day 1, day 8, and day 15 of a 3 week cycle. In some embodiments, Keytruda is administered once a week per three week cycle.

In certain embodiments, Keytruda is administered as an intravenous infusion at a dose of 2 mg/kg over 30 minutes every 3 weeks. In certain embodiments, Keytruda is administered as an intravenous infusion at a dose of 200 mg over 30 minutes every 3 weeks.

In some embodiments, a compound of Formula (I) (e.g., Compound 1), or a pharmaceutically acceptable salt thereof, is administered for the first week of a three week cycle (i.e., days 1-7 of a 21-day cycle) and Keytruda is administered on day 1 of the three week cycle. In some embodiments, a compound of Formula (I) (e.g., Compound 1), or a pharmaceutically acceptable salt thereof, is administered for the first week of a three week cycle (i.e., days 1-7 of a 21-day cycle) and Keytruda is administered on three days of the three week cycle. In some embodiments, a compound of Formula (I) (e.g., Compound 1), or a pharmaceutically acceptable salt thereof, is administered for the first week of a three week cycle (i.e., days 1-7 of a 21-day cycle) and Keytruda is administered on days 1, 8, and 15 of the three week cycle.

In some embodiments, a compound of Formula (I) (e.g., Compound 1), or a pharmaceutically acceptable salt thereof, is administered daily for a three week cycle (i.e., days 1-21 of a 21-day cycle) and Keytruda is administered on day 1 of the three week cycle. In some embodiments, a compound of Formula (I) (e.g., Compound 1), or a pharmaceutically acceptable salt thereof, is administered daily for a three week cycle (i.e., days 1-21 of a 21-day cycle) and Keytruda is administered on three days of the three week cycle. In some embodiments, a compound of Formula (I) (e.g., Compound 1), or a pharmaceutically acceptable salt thereof, is administered daily for a three week cycle (i.e., days 1-21 of a 21-day cycle) and Keytruda is administered on days 1, 8, and 15 of the three week cycle.

In certain embodiments, the second therapeutic agent used in combination with a compound of Formula (I), or a pharmaceutically acceptable salt thereof, is Yervoy. In certain embodiments, the methods described herein further comprise administering Yervoy as an intravenous infusion in a 3 week cycle (i.e., in 21-day cycles). In certain embodiments, Yervoy is administered as an intravenous infusion for multiple 3 week cycles (21-day cycles). In some embodiments, Yervoy is administered on day 1 of a 3 week cycle. In certain embodiments, Yervoy is administered on day 1, day 8, and day 15 of a 3 week cycle. In some embodiments, Yervoy is administered once a week per three week cycle.

In certain embodiments, Yervoy is administered as an intravenous infusion at a dose of 3 mg/kg over 90 minutes every 3 weeks for a total of 4 doses. In certain embodiments, Yervoy is administered as an intravenous infusion at a dose of 10 mg/kg over 90 minutes every 3 weeks for 4 doses, followed by 10 mg/kg every 12 weeks for up to 3 years.

In some embodiments, a compound of Formula (I) (e.g., Compound 1), or a pharmaceutically acceptable salt thereof, is administered for the first week of a three week cycle (i.e., days 1-7 of a 21-day cycle) and Yervoy is administered on day 1 of the three week cycle. In some embodiments, a compound of Formula (I) (e.g., Compound 1), or a pharmaceutically acceptable salt thereof, is administered for the first week of a three week cycle (i.e., days 1-7 of a 21-day cycle) and Yervoy is administered on three days of the three week cycle. In some embodiments, a compound of Formula (I) (e.g., Compound 1), or a pharmaceutically acceptable salt thereof, is administered for the first week of a three week cycle (i.e., days 1-7 of a 21-day cycle) and Yervoy is administered on days 1, 8, and 15 of the three week cycle.

In some embodiments, a compound of Formula (I) (e.g., Compound 1), or a pharmaceutically acceptable salt thereof, is administered daily for a three week cycle (i.e., days 1-21 of a 21-day cycle) and Yervoy is administered on day 1 of the three week cycle. In some embodiments, a compound of Formula (I) (e.g., Compound 1), or a pharmaceutically acceptable salt thereof, is administered daily for a three week cycle (i.e., days 1-21 of a 21-day cycle) and Yervoy is administered on three days of the three week cycle. In some embodiments, a compound of Formula (I) (e.g., Compound 1), or a pharmaceutically acceptable salt thereof, is administered daily for a three week cycle (i.e., days 1-21 of a 21-day cycle) and Yervoy is administered on days 1, 8, and 15 of the three week cycle.

### EXAMPLES

### Example 1: Exemplary Lipid-Based Formulations

Compound 1 (as a fee base) was formulated as a lipid-based formulation as an oral capsule dosage form. The capsules were packaged into a suitable sized clear Type I glass container sealed by a polypropylene screw cap lid with an aluminum foil liner. Formulation A and formulation B components are listed in Table 2 and table 3.

TABLE 2
Formulation A

| | | |
|---|---|---|
| | Quantity per | |
| Component | Capsule (mg) | Function |

Capsule Components

| | | |
|---|---|---|
| Compound 1 | 50.04 | Active |
| Propylene Glycol Monocaprylate¹ | 676.08 | Lipid |
| Macrogolglycerol Ricinoleate² | 173.88 | Lipid/Surfactant |
| Brown Hard Gelatin Capsule, size | 1 unit | Encapsulation |
| 00EL³ | | |
| Subtotal | 900 | — |

Capsule Sealing Components

| | | |
|---|---|---|
| Ethanol Absolute^{4, 5} | — | Sealing Solution |
| | | Solvent |
| Sterile Water for Irrigation^{4, 5} | — | Sealing Solution |
| | | Solvent |
| Total per Capsule | 900 | |

¹Capryol ™ 90.
²Kolliphor ® EL.
³Capsule contains gelatin, black iron oxide, red iron oxide, and yellow iron oxide.
⁴The quantity of materials used for the sealing procedure is not calculated per capsule.
⁵Evaporated during process therefore not included in final drug product weight.

TABLE 3
Formulation B

| | | |
|---|---|---|
| | Quantity per | |
| Component | Capsule (mg) | Function |

Capsule Components

| | | |
|---|---|---|
| Compound 1 | 50.00 | Active |
| Caprylic Acid | 750 | Lipid |
| Brown Hard Gelatin | 1 unit | Encapsulation |
| Capsule, size 00EL¹ | | |
| Subtotal | 800 | — |

Capsule Sealing Components

| | | |
|---|---|---|
| Ethanol Absolute^{2, 3} | — | Sealing Solution |
| | | Solvent |
| Sterile Water for | — | Sealing Solution |
| Irrigation^{2, 3} | | Solvent |
| Total per Capsule | 800 | |

¹Capsule contains gelatin, black iron oxide, red iron oxide, and yellow iron oxide.
²The quantity of materials used for the sealing procedure is not calculated per capsule.
³Evaporated during process therefore not included in final drug product weight.

### Example 2: Exemplary Powder for Reconstitution

Compound 1 (as a fee base) was formulated as a powder for reconstitution. The powder for reconstitution will be reconstituted as a suspension for oral administration with ORA-Blend® (a flavored Vehicle for Oral Suspension) immediately prior to dosing. The final powder for reconstitution formulation was packed into a 150 mL Type III amber glass bottle with a tamper evident polypropylene and enhanced polyethylene screw cap lid up to 48 hours prior to administration. The powder for reconstitution was prepared as unit doses ranging from a low strength 100 mg dose to high strength 800 mg dose. The powder for reconstitution components are listed in Table 4.

TABLE 4

| | | | |
|---|---|---|---|
| | Quantity per | Quantity per | |
| | 100 mg | 800 mg | |
| Component | Bottle (mg) | Bottle (mg) | Function |

Powder for Reconstitution Components

| | | | |
|---|---|---|---|
| Compound 1 | 100 | 800 | Active |
| HPMC-AS | 300 | 2400 | Polymer |

Spray Solution Components

| | | | |
|---|---|---|---|
| Absolute Ethanol² | — | — | Spray solution |
| | | | solvent |
| Sterile Water for | — | — | Spray solution |
| Irrigation² | | | solvent |
| Total per weight | 400¹ | 3200¹ | — |

¹Total solid content.
²Removed from the powder for reconstitution during the drying process.

### Example 3: Exemplary Suspension Formulation

Suspensions comprising Compound 1 (HCl salt) were prepared. The suspension components are listed in Table 5.

TABLE 5

| | Amount, | | Amount per 500 mL | |
|---|---|---|---|---|
| Component | mg/mL | | Batch, g | |
| Compound 1•HCl salt | 17.82 | (16.00) | 8.91 | (8.00) |
| (As Compound 1 free base) | | | | |
| ORA-Blend ® | q.s. | | q.s. | |
| To make | 1.0 mL | (1.16 g) | 500 mL | (579 g) |

### Example 4: Stability of Lipid-Based, Powder for Reconstitution, and Suspension Formulations

The appearance was assessed visually. The assay, identity, and related substances were assessed by a reversed phase gradient UPLC method as detailed in the table below:

| Component | Condition | | |
|---|---|---|---|
| Column | Waters Acquity UPLC CSH C18 Column | | |
| | (3.0 mm × 150 mm) 1.7 μm or | | |
| | suitably validated alternative | | |
| Mobile Phases | Mobile Phase A: 0.05% Formic acid | | |
| | in water | | |
| | Mobile Phase B: 0.05% Formic acid | | |
| | in acetonitrile | | |
| | Time | % Mobile | % Mobile |
| | (min) | Phase A | Phase B |
| Gradient | 0.00 | 80 | 20 |
| | 23.00 | 50 | 50 |
| | 25.00 | 5 | 95 |
| | 30.00 | 5 | 95 |
| | 30.10 | 80 | 20 |
| | 35.00 | 80 | 20 |

| | |
|---|---|
| Flow Rate | 0.7 mL/min |
| Run Time | 35 min |
| Injection Volume | 2 μL |
| Needle Wash | 100% Methanol |
| Column Temperature | 80° C. |
| Auto Sampler | Ambient (20° C.) |
| Temperature | |
| Detection | 248 nm |

The disintegration test was performed according to Ph. Eur. Monograph 2.9.1. The media used for the test was 0.1M hydrochloric acid.

The residual solvent was assessed by a head space gas chromatography method with flame ionization detection as detailed in the table below:

| Component | Condition | | |
|---|---|---|---|
| Column | Agilent DB-624 column (30 m × 0.32 mm ID) | | |
| | 1.8 μm or suitably validated alternative | | |
| Detection | Flame Ionization | | |
| Injector Temperature | 140° C. | | |
| Split Control mode | Ratio | | |
| Split Ratio | 2.1 | | |
| Purge Mode | Fixed | | |
| Purge Flow | 5 mL/min | | |
| Column Program | 40° C. for 20 min | | |
| | 10° C./min to 240° C. | | |
| | Hold at 240° C. for 20 min | | |
| Run Time | 60 min | | |
| Lineal Velocity | 35 cm/sec | | |
| Carrier Gas | Helium | | |
| Detector Temperature | 250° C. | | |
| Hydrogen Flow | 45 mL/min | | |
| Compressed Air Flow | 450 mL/min | | |
| Detector Active | 0.0-60.0 min | | |
| Filter Time Constant | 200 | | |
| Flameout Threshold | off | | |
| | Parameter | Setting(s) | |
| GC Headspace | Oven Temperature | 80° | C. |
| Conditions | Loop Temperature | 85° | C. |
| | Transfer Line Temperature | 85° | C. |
| | GC Cycle Time | 70 | min |
| | Venting Time | 5 | sec |
| | Flow Rate (Headspace Unit) | 20 | mL/min |
| | Column Pressure | 96.5 | kPa |
| | Incubation Time | 60 | min |
| | Pressurization Time | 2.0 | min |
| | Withdraw Time | 0.1 | min |

The method for determining water content was performed according to USP<921>. A coulometric Karl Fisher titration method was used with a Metrohm 831 KF Coulometer or equivalent and a Metrohm Model 832 or Model 860 Thermoprep oven or equivalent.

Stability data for exemplary lipid-based formulations A and B (See example 1) are presented in tables 6A, 6B, 7A, and 7B.

TABLE 6A
Formulation A stability data

| | | | | Identity | | | |
|---|---|---|---|---|---|---|---|
| | | | Appearance | Retention time of the | | | |
| | | | Brown Capsule with | Compound 1 peak in | | | |
| | | | evidence of a sealing | the sample solution | | | |
| | | | band around the | must be within ±0.5 | | | |
| | | | circumference of | minutes of the average | | | |
| | | | the capsule joint, | retention time of the | Assay | | Uniformity of |
| Condition | | Days | otherwise free from | compound 1 reference | 90-111% | Disintegration | Dosage Units |
| Acceptance Criteria | | | visual defects | standard peak | nominal | Report results | AV ≤ 15.0 |
| N/A | | 0 | Complies | Complies | 99.9% | <10 minutes | 2.3 |
| 2-8° | C. | 7 | Complies | ND | 99.5% | <10 minutes | ND |
| 15-25° | C. | 7 | Complies | ND | 99.2% | <10 minutes | ND |

ND: not determined

TABLE 6B
Formulation A stability data

| | | | A4 | A1 | | | | | |
|---|---|---|---|---|---|---|---|---|---|
| Condition | | Days | (RRT0.98) | (RRT1.12) | RRT0.71 | RRT0.87 | RRT1.04 | RRT1.26 | Total |
| Acceptance Criteria | | | ≤0.3% | ≤2.0% | ≤0.6% | ≤0.6% | ≤0.6% | ≤0.6% | ≤3.0% |
| N/A | | 0 | 0.05% | 0.63% | <LOQ | 0.09% | 0.06% | 0.07% | 0.9% |
| 2-8° | C. | 7 | 0.06% | 0.71% | 0.09% | 0.12% | <LOQ | 0.07% | 1.1% |
| 15-25° | C. | 7 | 0.06% | 0.81% | 0.07% | 0.12% | 0.08% | 0.07% | 1.3% |

TABLE 7A
Formulation B stability data

| | | | | Identity | | | |
|---|---|---|---|---|---|---|---|
| | | | Appearance | Retention time of the | | | |
| | | | Brown Capsule with | Compound 1 peak in | | | |
| | | | evidence of a sealing | the sample solution | | | |
| | | | band around the | must be within ±0.5 | | | |
| | | | circumference of | minutes of the average | | | |
| | | | the capsule joint, | retention time of the | Assay | | Uniformity of |
| Condition | | Days | otherwise free from | compound 1 reference | 90-111% | Disintegration | Dosage Units |
| Acceptance Criteria | | | visual defects | standard peak | nominal | Report results | AV ≤ 15.0 |
| N/A | | 0 | Complies | Complies | 100.6% | <30 minutes | 1.4 |
| 2-8° | C. | 7 | Complies | ND | 100.4% | <30 minutes | ND |
| 15-25° | C. | 7 | Complies | ND | 96.1% | <30 minutes | ND |

ND: not determined

TABLE 7B
Formulation B stability data

| | | | A4 | A1 | | | | |
|---|---|---|---|---|---|---|---|---|
| Condition | | Days | (RRT0.98) | (RRT1.12) | RRT0.62 | RRT0.71 | RRT0.72 | RRT0.77 |
| Acceptance Criteria | | | ≤0.3% | ≤2.0% | ≤0.6% | ≤0.6% | ≤0.6% | ≤0.6% |
| N/A | | 0 | 0.05% | 0.98% | <LOQ | 0.07% | 0.07% | <LOQ |
| 2-8° | C. | 7 | <LOQ | 0.98% | <LOQ | <LOQ | <LOQ | <LOQ |
| 15-25° | C. | 7 | <LOQ | 3.81% | 0.06% | <LOQ | <LOQ | 0.28% |
| Condition | | Days | RRT0.78 | RRT0.87 | RRT1.03 | RRT1.21 | RRT1.26 | Total |
| Acceptance Criteria | | | ≤0.6% | ≤0.6% | ≤0.6% | ≤0.6% | ≤0.6% | ≤3.0% |
| N/A | | 0 | <LOQ | 0.11% | <LOQ | <LOQ | 0.07% | 1.4% |
| 2-8° | C. | 7 | <LOQ | 0.09% | 0.07% | <LOQ | 0.07% | 1.2% |
| 15-25° | C. | 7 | 0.28% | 0.08% | 0.07% | 0.10% | 0.06% | 4.7% |

Stability data for an exemplary powder for reconstitution (See example 2) are presented in table 8A and 8B.

TABLE 8A
Powder for Reconstitution stability data

| | | | | Identity | | | |
|---|---|---|---|---|---|---|---|
| | | | | Retention time of the | | | |
| | | | | Compound 1 peak in | | | |
| | | | | the sample solution | | | |
| | | | | must be within ±0.5 | | Residual | |
| | | | Appearance | minutes of the average | | solvent | |
| | | | White to | retention time of the | Assay | NMT | Water |
| Condition | | Days | off-white | compound 1 reference | 90-111% | 5000 ppm | Content |
| Acceptance Criteria | | | powder | standard peak | nominal | ethanol | Report result |
| N/A | | 0 | Complies | Complies | 96.6% | 44 ppm | 1.1% |
| 2-8° | C. | 7 | Complies | ND | 97.1% | ND | 0.9% |
| 15-25° | C. | 7 | Complies | ND | 96.7% | ND | 1.0% |

TABLE 7B
Powder for Reconstitution stability data

| | | | A4 | A1 | | | | |
|---|---|---|---|---|---|---|---|---|
| Condition | | Days | (RRT0.98) | (RRT1.12) | RRT0.87 | RRT1.05 | RRT1.26 | Total |
| Acceptance Criteria | | | ≤0.3% | ≤2.0% | ≤0.6% | ≤0.6% | ≤0.6% | ≤0.6% |
| N/A | | 0 | 0.05% | 0.64% | 0.08% | 0.07% | 0.08% | 0.92% |
| 2-8° | C. | 7 | 0.05% | 0.75% | 0.08% | 0.10% | 0.07% | 1.1% |
| 15-25° | C. | 7 | 0.05% | 0.77% | 0.09% | 0.12% | 0.07% | 1.1% |

A summary of the stability studies for an exemplary suspension formulation (See example 3) are presented in table 9.

TABLE 9A
Suspension stability data

| | | Storage | |
|---|---|---|---|
| | | Conditions | Time |
| Study Type | Container | Evaluated, ° C. | Completed |
| Stability and | None: bulk | 5 and 25 | 24 hours |
| Homogeneity | container | | |
| Stability | Oral dispenser, | −20 and 25 | 6 hours at 25° C. |
| (laboratory batch) | 20 mL and 60 mL | | 7 days at −20° C. |
| Stability | Oral dispenser, | 25¹ | 24 hours |
| (demonstration | 20 mL and 60 mL | | |
| batch) | | | |

¹Samples frozen at −20° C. or lower until analyzed

### Example 5: Pharmacokinetic Study

Compound 1 was administered orally by gavage tubes once to male beagle dogs. Blood was collected via the jugular vein at the designated time points into collection tubes containing K2EDTA and stored on ice until processed by centrifugation. The resulting plasma was transferred into a 96-well container, and stored in a freezer set to maintain approximately −80° C.

Three exemplary formulations were compared against a reference formulation (2% HPMC/0.2% Tween® 80) in a dog pharmacokinetic study (dose 10 mg/kg).

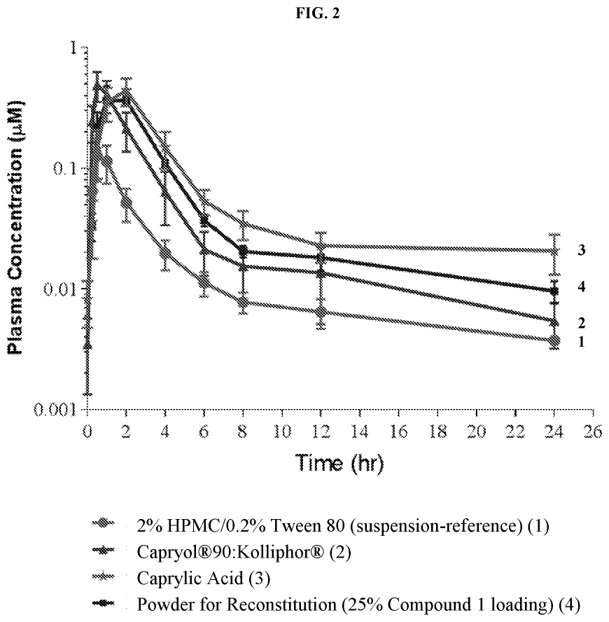

The Area Under the Curve data and Cmax data are shown in table 10. The graphs are shown in FIG. 2.

TABLE 10

| | | | | Fold over | |
|---|---|---|---|---|---|
| | | | | suspension | |
| | | AUC_{inf} | Cₘₐₓ | reference | |
| Formulation | Fasted/Fed | (μM*h) | (μM) | AUC | Cₘₐₓ |
| Reference¹ | Fed | 0.363 | 0.130 | — | — |
| Capryol ® 90: Kolliphor ® | Fasted | 1.27 | 0.490 | 3.5 | 3.8 |
| Caprylic Acid | Fasted | 2.31 | 0.442 | 6.4 | 3.4 |
| Powder for reconstitution | Fasted | 1.61 | 0.437 | 4.4 | 3.4 |
| (HPMC) | | | | | |

¹2% HPMC/0.2% Tween ® 80 (suspension)

Exposures of Compound 1 following single administrations of Compound 1 in different formulations (in fasted dogs) was also evaluated.

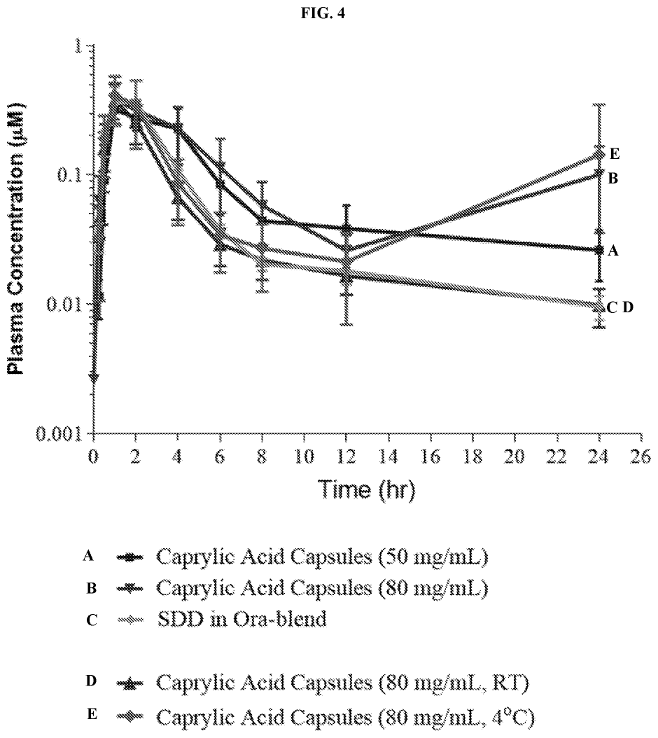

The Area Under the Curve data, Cmax, and Tmax data are shown in table 11. The graphs are shown in FIG. 4.

TABLE 11

| | | Original | | | | |
|---|---|---|---|---|---|---|
| | | Solubility | AUC_{inf} | AUC₀₋₂₄ | Cₘₐₓ | Tₘₐₓ |
| # | Formulation | (mg/mL) | (μM*h) | (μM*h) | (μM) | (hr) |
| 1 | Caprylic acid in capsules | 50 | 2.35 | 1.92 | 0.396 | 1.0~4.0 |
| 2 | Caprylic acid in capsules | 80 | 2.06 | 2.51 | 0.479 | 1.0~4.0 |
| 3 | HPMC SDD Suspension | 25% | 1.86 | 1.56 | 0.350 | 1.0~4.0 |
| 4 | Caprylic acid in capsules | 80 at RT | 1.46 | 1.19 | 0.408 | 0.5~2.0 |
| 5 | Caprylic acid in capsules | 80 at 4° C. | 2.00 | 1.43 | 0.446 | 1.0~2.0 |

Tested formulations:
#1: Compound 1, caprylic acid in Capsule, 50 mg/mL, 120 mg/animal,
#2: Compound 1, caprylic acid in Capsule, 80 mg/mL, 120 mg/animal,
#3: Compound 1, HPMC SDD, 125 mg/animal,
#4: Compound 1, caprylic acid in Capsule, 80 mg/mL, 126 mg/animal, (RT),
#5: Compound 1, caprylic acid in Capsule, 80 mg/mL, 126 mg/animal, (4° C.).

### Example 6: Synthesis of Compound 1

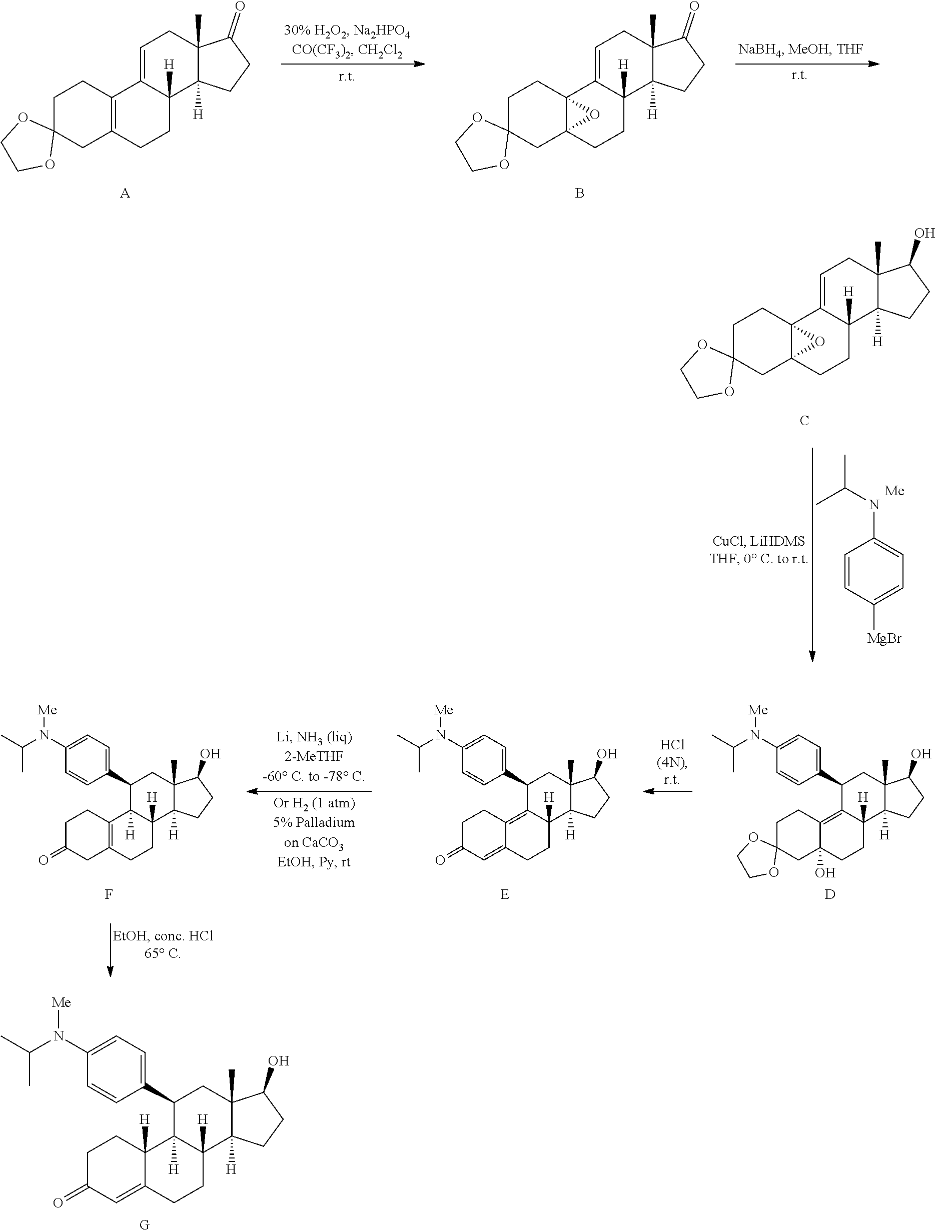

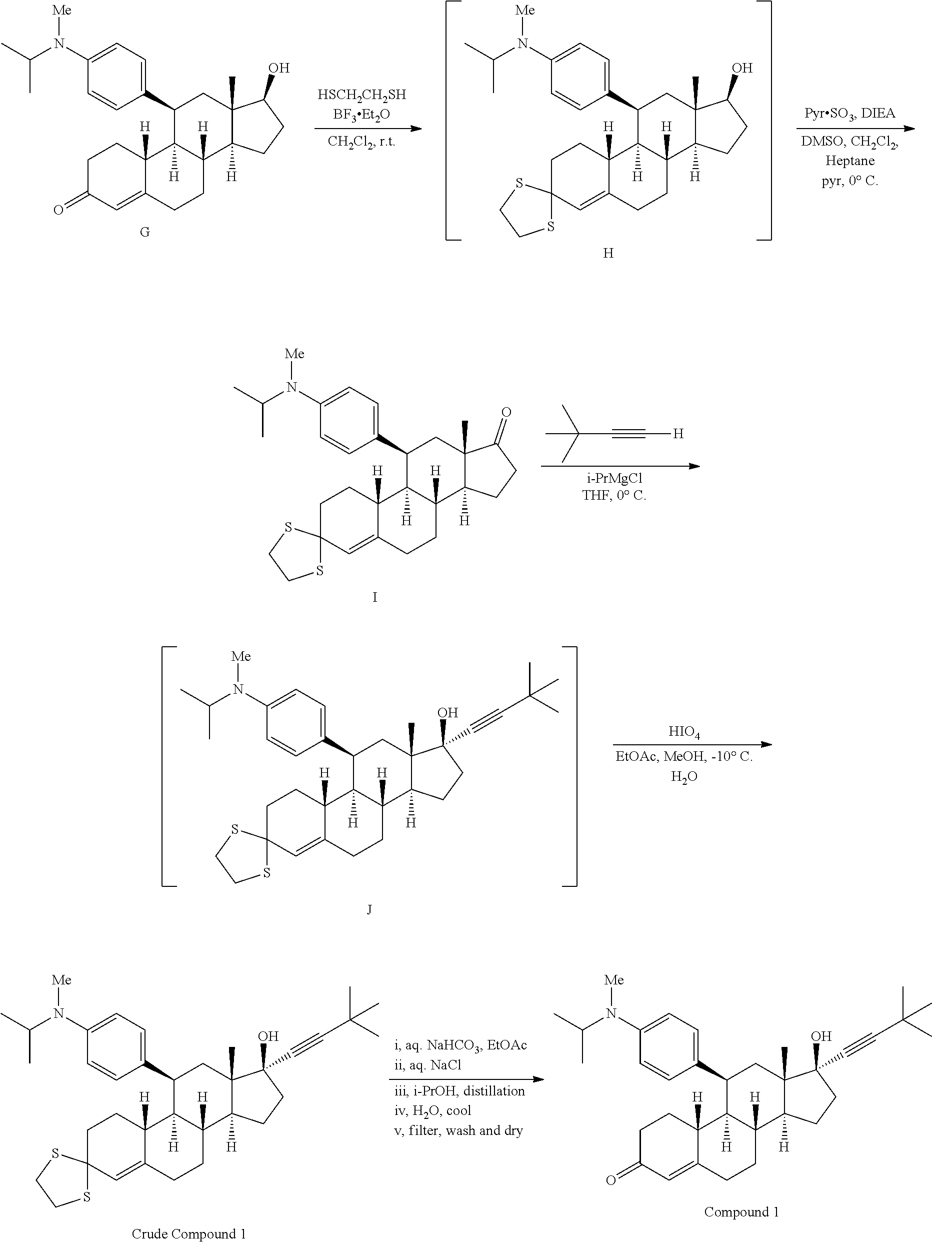

### Manufacture of Intermediate G

To a solution of Intermediate E (18 g, 42.9 mmol) in EtOH (144 mL) and pyridine (36 mL) was added 5% Pd on CaCO₃ (1.8 g). The reaction mixture was stirred for 6 h at room temperature under H₂ atmosphere. The mixture was filtered. The filtrate was concentrated under reduced pressure to afford crude Intermediate F (20.8 g), which was used directly without further purification.

The crude Intermediate F (20.8 g) was added to a mixture of EtOH (40 mL) and 12N aqueous HCl (120 mL). The resulting mixture was stirred for 8 h at 65° C. The mixture was concentrated under reduced pressure to remove EtOH. The resulting solution was basified to pH=8-9 with 4N aqueous NaOH and then extracted with EtOAc (3×150 mL). The combined organic layers were washed with water (100 mL) and brine (3×100 mL), dried over anhydrous Na₂SO₄ and concentrated under reduced pressure. The residue was purified by column chromatography on silica gel (petroleum ether/acetone=8:1 to 4:1) followed by recrystallization from petroleum ether/EtOAc (3:1) to afford Intermediate G (7.5 g, 42% yield over two steps) as a white solid.

### Manufacture of Intermediate I

1,2-Ethanedithiol was added to a solution of G in dichloromethane. The reaction mixture was cooled, boron trifluoride etherate (BF₃.Et₂O) was added and the mixture was stirred at room temperature. Upon reaction completion, the mixture was cooled and aqueous sodium hydroxide was added. The product was partitioned into dichloromethane; the organic phase was washed with aqueous sodium chloride and aqueous citric acid before being dried with magnesium sulfate. Dimethyl sulfoxide and N,N-diisopropylethylamine were added to the dichloromethane solution and cooled; a solution of pyridine.SO₃ and pyridine in dimethyl sulfoxide was then added slowly to the cooled reaction mixture. After reaction completion, the product mixture was washed twice with aqueous citric acid solution followed by aqueous sodium chloride. The dichloromethane phase was concentrated and recrystallized from dichloromethane/methanol. The solid (I) was filtered, washed with dichloromethane/methanol, and dried under vacuum at approximately 45° C.

### Manufacture of Crude Compound 1

3,3-Dimethyl-1-butyne was added to a cooled solution of isopropylmagnesium chloride in tetrahydrofuran and the mixture was stirred with warming. A slurry of I in tetrahydrofuran was added to the cooled reaction mixture. Upon reaction completion, the reaction mixture was quenched with aqueous ammonium chloride and ethyl acetate was added. The organic phase was separated, washed with aqueous sodium chloride and concentrated. After removal of tetrahydrofuran by distillation, ethyl acetate and methanol were added and the solution was cooled. A solution of aqueous periodic acid was then added with cooling. The reaction mixture was then quenched with a cold aqueous solution of sodium sulfite. Ethyl acetate was added and the organic phase was then washed with aqueous sodium bicarbonate then aqueous sodium chloride. The crude Compound 1 was then stirred with activated charcoal (Nuchar Aquaguard), filtered through a pad of Celite followed by a wash of ethyl acetate.

### Manufacture of Compound 1

The ethyl acetate solution was concentrated followed by the addition of isopropanol. The concentration/isopropanol charge was repeated. The isopropanol solution was filtered through an in-line filter (polish) then concentrated. Purified water was added to the isopropanol solution at approximately 70° C. The batch was then cooled to approximately 50° C. and held to effect crystallization. Purified water was added and the mixture cooled to approximately 0° C. The product was filtered and washed with a mixture of isopropanol/water at approximately 5° C. Compound 1 was dried under vacuum at approximately 45° C.

### Example 7: Capsule Formulation

The solubility of Compound 1 was assessed in a number of lipids including those listed in Table 12.

TABLE 12
Lipids Used for Solubility Screen

| Lipid | Solubility | |
|---|---|---|
| Kolliphor EL (PEG-35 Castor Oil USP) | <40 | mg/mL |
| Capmul MCM (Glyceryl monocaprylcaprate, | <40 | mg/mL |
| Type I, NF) | | |
| Tween 80 (polysorbate 80 NF) | <40 | mg/mL |
| Maisine CC (Caprylic/capric | <40 | mg/mL |
| triglyceride NF) | | |
| Tween 20 (Polysorbate 20 NF) | <40 | mg/mL |
| Capryol 90 (Propylene glycol | 40 | mg/mL |
| monocaprylate, Type II, NF) | | |
| Labrasol ALF (Caprylocaproyl Polyoyl-8 | 40 | mg/mL |
| glycerides, NF) | | |
| Capryol 90 (Propylene glycol monocaprylate, | 50 | mg/mL¹ |
| Type II, NF) + 5% Poloxamer P188 USP | | |

¹particulate seen after 24 hours

Initial work indicated that Compound 1 could achieve a dose of 50 mg Compound 1 in a formulation with a capsule fill weight of 800-900 mg. A hard gelatin capsule shell was used. These were identified at Formulations A & B whose compositions are provided in Table 13. These capsules were sealed to minimize leaking.

TABLE 13
Compositions of Formulations A and B

| | Amount per capsule, | |
|---|---|---|
| | mg | |
| Ingredient | Formulation A | Formulation B |
| Compound 1 | 50 | 50 |
| Capryol 90 (Propylene glycol | 676.08 | 0 |
| monocaprylate, Type II, NF) | | |
| Kolliphor EL (PEG-35 Castor Oil USP) | 173.88 | 0 |
| Caprylic acid | 0 | 750.00 |
| Capsule Fill Weight, mg | 900.0 | 800.0 |

Compound 1 in caprylic acid upon extended stability showed formation of primary oxidative N-demethylation product (A1, see Table 1), also a known metabolite of Compound 1, at greater than 4% after about 4-6m.

Two antioxidants, α-tocopherol and ascorbyl palmitate, were evaluated to minimize the oxidation. Use of α-tocopherol alone improved Compound 1 stability as measured by the formation of A1 (see Table 1). When ascorbyl palmitate was combined with α-tocopherol, further stability improvement was observed. Table 14 summarizes the compositions of the Compound 1 capsule matrix evaluated and Table 15 summarizes the effect of α-tocopherol and ascorbyl palmitate as stabilizers on the stability of Compound 1. The combination of α-tocopherol and ascorbyl palmitate as stabilizers in the formulation substantially suppresses the formation of A1 (see Table 1).

TABLE 14
Compositions Evaluated for Effect of Stabilizers

| | Formulation, mg (mg per 800 mg composition) | | | | | | | | | |
|---|---|---|---|---|---|---|---|---|---|---|
| Ingredient | A | | B | | C | | D | | E | |
| Compound 1 | 58.9 | (44.5) | 58.9 | (44.3) | 58.9 | (43.8) | 80.0 | (59.0) | 100.0 | (73.4) |
| Caprylic acid, NF | 1000 | (755.5) | 1000 | (757.0) | 1000 | (743.6) | 1000 | (737.1) | 1000 | (723.8) |
| α-Tocopherol | 0.0 | (0) | 5.0 | (3.8) | 5.0 | (3.7) | 5.0 | (3.7) | 5.0 | (3.6) |
| Ascorbyl palmitate | 0.0 | (0) | 0.0 | (0) | 12.0 | (8.9) | 0.3 | (0.2) | 0.3 | (0.2) |

TABLE 15
Effect of Stabilizer on Stability of Compound 1 Capsule
Matrix as Measured by A1 (see Table 1) Concentration

| Formulation | 0 | 7 d | 14 d | 28 d | 56 d | 3 m |
|---|---|---|---|---|---|---|
| A, % | 0 | 2.46 | 2.7 | 4.00 | 5.30 | NT |
| B, % | 0.61 | 0.90 | 0.95 | 1.31 | 1.68 | NT |
| C, % | 0.63 | 0.69 | 0.62 | 0.63 | 0.65 | 0.63 |
| D, % | 0.66 | 0.65 | 0.65 | 0.70 | NT | NT |
| E, % | 0.64 | 0.70 | 0.83 | 0.75 | NT | NT |

NT: not tested

On the basis of these data, the current formulation summarized in Table 16 was developed and selected for use in the clinical study.

TABLE 16
Clinical Study Composition

| | Amount per |
|---|---|
| Ingredient/Component | Capsule, mg |
| Compound 1 (based on 100% purity) | 80.00 |
| α-Tocopherol, USP | 4.10 |
| L(+) Ascorbyl palmitate, NF | 0.25 |
| Caprylic acid, NF | 735.65 |
| Hard gelatin capsule, clear, 00EL | One |

Capsule sealant

| | |
|---|---|
| Polysorbate 80, NF | q.s. |
| Gelatin, USP | q.s. |
| Purified water, USP | Removed during drying |
| | leaving residual amount |

### Example 8: Solubility Study

This study was carried out to evaluate the solubility of Compound 1 (freebase form) in a variety of dosing vehicles to identify excipient classes that maximize drug solubility. A total of 20 vehicles which were selected and agreed with the customer were used in this study. The results are shown in Table 17.

### Sample Preparation:

Compound 1 was added to approximately 1 gram of the excipients. After the initial addition of Compound 1, the mixtures were shaken in a temperature-controlled vortex mixer at 25° C. (40° C. for vehicles that were semi-solid at room temperature and 50° C. for solid vehicles) then examined for solid residues. If dissolution was observed during the mixing time, additional Compound 1 was added until no further dissolution was observed.

After shaking for a total of five days, the suspensions were filtered using a centrifuge tube with 0.45 PVDF membrane filter (Millipore Durapore®). The filtrate was weighed in to a 25-mL volumetric flask and diluted to volume with the diluent solution (90:10% v/v IPA/Acetonitrile) for a determination of Compound 1 concentration via High Performance Liquid Chromatography (HPLC). Residual solids were inspected by PXRD to determine the solid form.

### Powder X-Ray Diffraction (PXRD):

PXRD diffractograms were acquired using Bruker D8 Advance diffractometer using a Ni-filtered Cu Kα (40 kV/40 mA) radiation and a step size of 0.02° 2θ between 4° to 40° 2θ. Samples were mounted on Si zero-background wafers.

### High-Performance Liquid Chromatography:

Quantitative determination of the amount of API dissolved was performed using reverse-phase HPLC analyses on an Agilent 1290 Infinity II UHPLC system equipped with a Diode Array Detector (DAD).

### Chromatographic Conditions

Agilent 1290 Infinity II UHPLC system equipped with a Diode Array Detector (DAD)
Mobile Phase A: 0.05% Formic acid in water
Mobile Phase B: 0.05% Formic acid in acetonitrile

### Column: Acquity UPLC CSH C18, 150×3.0 mm, 1.7 μm

### Column Temperature: 60° C.

Flow Rate: 0.7 mL/min, gradient

### Detection: 248 nm

### Gradient:

| Time (minutes) | Mobile Phase A (%) | Mobile Phase B (%) |
|---|---|---|
| 0 | 80 | 20 |
| 23 | 50 | 50 |
| 25 | 5 | 95 |
| 30 | 5 | 95 |
| 30.1 | 80 | 20 |
| 35 | 80 | 20 |

TABLE 17

| Excipient | Description | HLB | Solubility (mg/g) |
|---|---|---|---|
| PEG 400 | Hydrophilic Solvent | N/A | 14.5 |
| Propylene Glycol | Hydrophilic Solvent | N/A | 12.1 |
| Glycerin | Hydrophilic Solvent | N/A | 0.1 |
| Diethyl glycol monoethyl ether | Hydrophilic Solvent | N/A | 130.3 |
| Triacetin | Lipophilic solvent (SCT) | N/A | 21.9 |
| Medium chain triglycerides | Lipophilic solvent (MCT) | N/A | 26.1 |
| Sorbitan monooleate | Low HLB surfactant | 4.3 | 19.5 |
| Glyceryl monolinoleate | Low HLB surfactant | 1 | 41.2 |
| Propylene glycol monolaurate | Low HLB surfactant | 3 | 68.4 |
| Glyceryl caprylate/caprate | Low HLB surfactant | 5 | 79.0 |
| Propylene glycol monocaprylate | Low HLB surfactant | 5 | 139.6 |
| Sorbitan Monolaurate | Middle HLB surfactant | 9 | 34.1 |
| Lauroyl polyoxyl-6 glycerides | Middle HLB surfactant | 9 | 40.8 |
| Polyethylene glycol sorbitan monolaurate | High HLB surfactant | 16 | 26.1 |
| Caprylocaproyl polyoxyl-8 glycerides | High HLB surfactant | 12 | 42.4 |
| Vitamin E TPGS | High HLB surfactant | 13 | 8.3 |
| Lauroyl polyoxyl-32 glycerides | High HLB surfactant | 14 | 39.2 |
| Polyethylene glycol monostearate | High HLB surfactant | 16 | 34.2 |
| Octanoic acid (Caprylic acid) | Medium chain fatty acid | N/A | 199.2 |
| Decanoic acid (Capric acid) | Medium chain fatty acid | N/A | 201.0 |

An empirical parameter commonly used to characterize the relative hydrophilicity and hydrophobicity of non-ionic amphiphilic compounds is the hydrophilic-lipophilic balance (the “HLB” value). Surfactants with lower HLB values are more hydrophobic, and have greater solubility in oils, whereas surfactants with higher HLB values are more hydrophilic, and have greater solubility in aqueous mediums.

It should be appreciated that the HLB value of a surfactant is merely a rough guide generally used to enable formulation of industrial, pharmaceutical and cosmetic emulsions. For many important surfactants, including several polyethoxylated surfactants, it has been reported that HLB values can differ by as much as about 8 HLB units, depending upon the empirical method chosen to determine the HLB value (Schott, J. Pharm. Sciences, 79(1), 87-88 (1990)).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A lipid-based formulation comprising:
(a) a lipid; and
(b) a compound of Formula (I), or a pharmaceutically acceptable salt thereof: 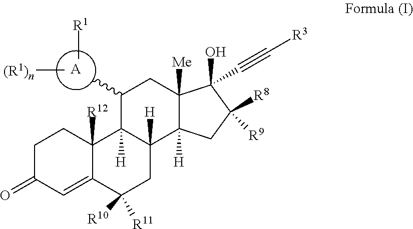
wherein
ring A is a heteroaryl or aryl;
R¹ is —NR^{4a}R^{5a};
each R² is independently —NR⁴R⁵, halo, —OR⁶, —OH, optionally substituted alkyl, or haloalkyl;
R³ is optionally substituted C₂₋₈ alkyl, halo, haloalkyl, optionally substituted cycloalkyl, optionally substituted cycloalkylalkyl, optionally substituted heterocycloalkyl, optionally substituted heterocycloalkylalkyl, optionally substituted heteroalkyl, optionally substituted aryl, optionally substituted heteroaryl, —Si(R⁶)₃, —OR⁶, or —S(O)₂R⁷;
R^{4a} is C₂₋₈ alkyl, optionally substituted cycloalkyl, optionally substituted aryl, optionally substituted heterocycloalkyl, or optionally substituted heteroaryl;
R^{5a} is —H, optionally substituted alkyl, or haloalkyl;
or R^{4a} and R^{5a} are taken together with the N atom to which they are attached to form an optionally substituted heterocycloalkyl;
R⁴ and R⁵ are each independently —H, optionally substituted alkyl, or haloalkyl;
or R⁴ and R⁵ are taken together with the N atom to which they are attached to form an optionally substituted heterocycloalkyl;
each R⁶ is independently optionally substituted alkyl or haloalkyl;
R⁷ is optionally substituted alkyl or haloalkyl;
R⁸ and R⁹ are each independently —H, optionally substituted alkyl, haloalkyl, or halo;
R¹⁰ and R¹¹ are each independently —H, optionally substituted alkyl, halo, or haloalkyl;
R¹² is hydrogen, optionally substituted alkyl, haloalkyl, hydroxy, or halo;
n is 0, 1, or 2.

2. The lipid-based formulation of claim 1, wherein R¹² is C₁₋₆ alkyl or hydrogen.

3. The lipid-based formulation of claim 1 or 2, wherein R¹² is methyl.

4. The lipid-based formulation of claim 1 or 2, wherein R¹² is H.

5. The lipid-based formulation of any one of claims 1-4, wherein ring A is phenyl.

6. The lipid-based formulation of any one of claims 1-5, wherein R^{4a} is C₂₋₈ alkyl.

7. The lipid-based formulation of any one of claims 1-6, wherein R^{4a} is C₃₋₆ alkyl.

8. The lipid-based formulation of any one of claims 1-7, wherein R^{4a} is C₂₋₄ alkyl.

9. The lipid-based formulation of any one of claims 1-8, wherein R^{4a} is ethyl, i-propyl, or t-butyl.

10. The lipid-based formulation of any one of claims 1-9, wherein R^{5a} is —H, optionally substituted alkyl, or haloalkyl.

11. The lipid-based formulation of any one of claims 1-10, wherein R^{5a} is —H or alkyl.

12. The lipid-based formulation of any one of claims 1-11, wherein R^{5a} is C₁₋₆ alkyl.

13. The lipid-based formulation of any one of claims 1-12, wherein n is 0 or 1.

14. The lipid-based formulation of any one of claims 1-13, wherein each R² is independently halo.

15. The lipid-based formulation of any one of claims 1-14, wherein R³ is optionally substituted C₂₋₈ alkyl, haloalkyl, or optionally substituted cycloalkyl.

16. The lipid-based formulation of any one of claims 1-15, wherein R³ is C₄₋₈ alkyl.

17. The lipid-based formulation of any one of claims 1-16, wherein R⁸ and R⁹ are —H.

18. The lipid-based formulation of any one of claims 1-17, wherein R¹⁰ and R¹¹ are each —H.

19. The lipid-based formulation of any one of claims 1-18, wherein the compound has the structure of Formula (Ia):

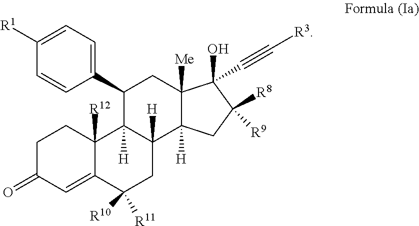

20. The lipid-based formulation of claim 1, wherein the compound is: 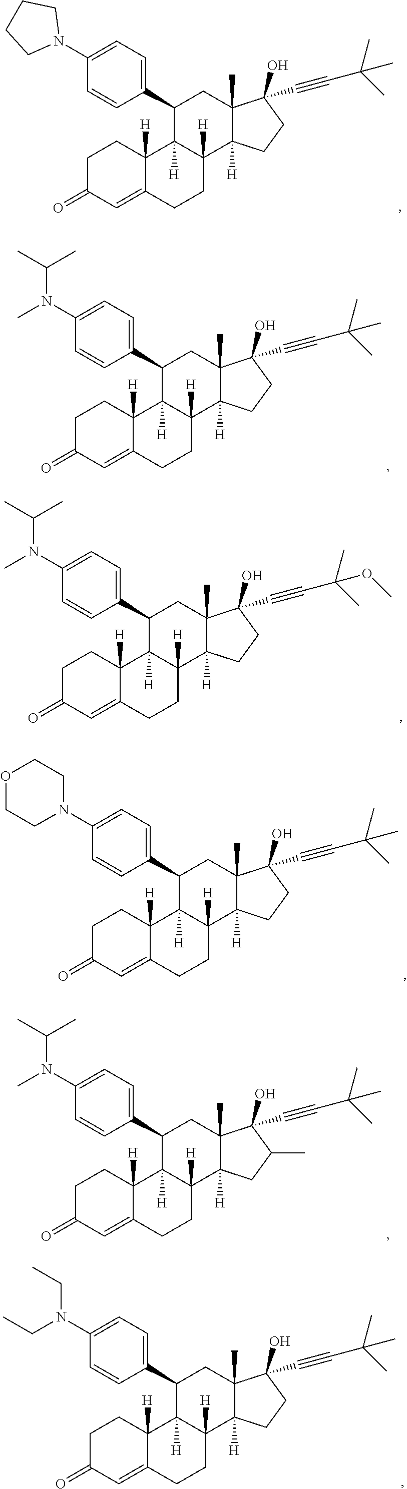 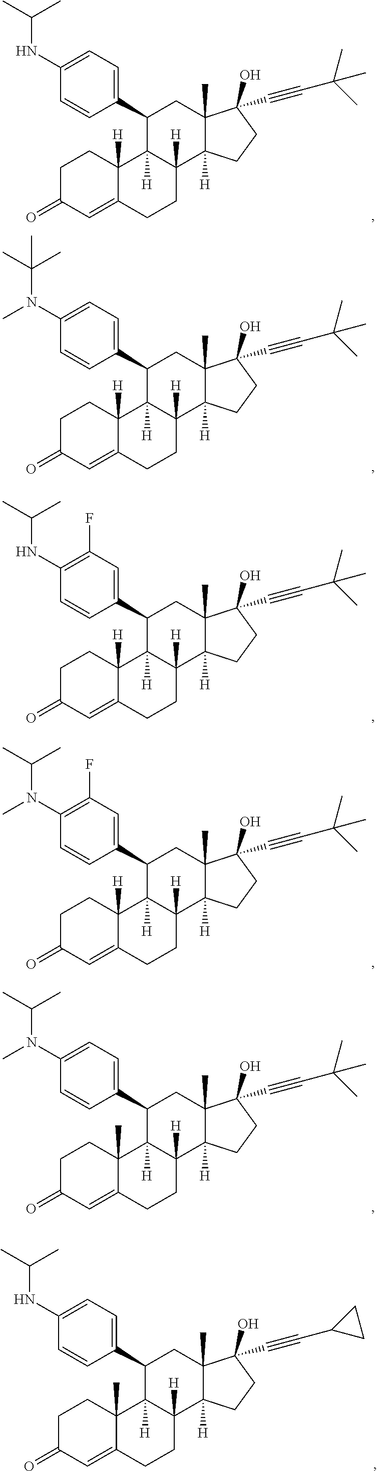 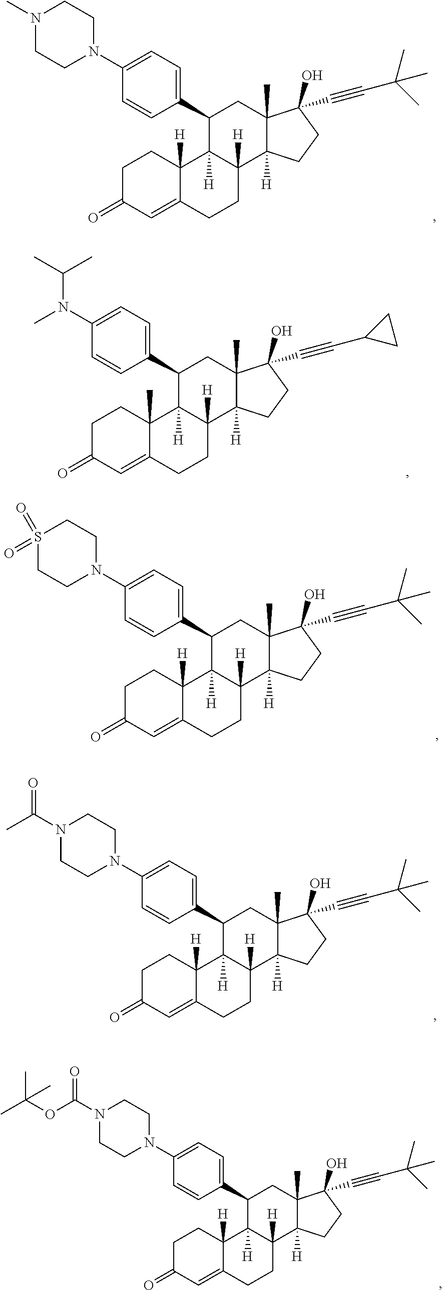 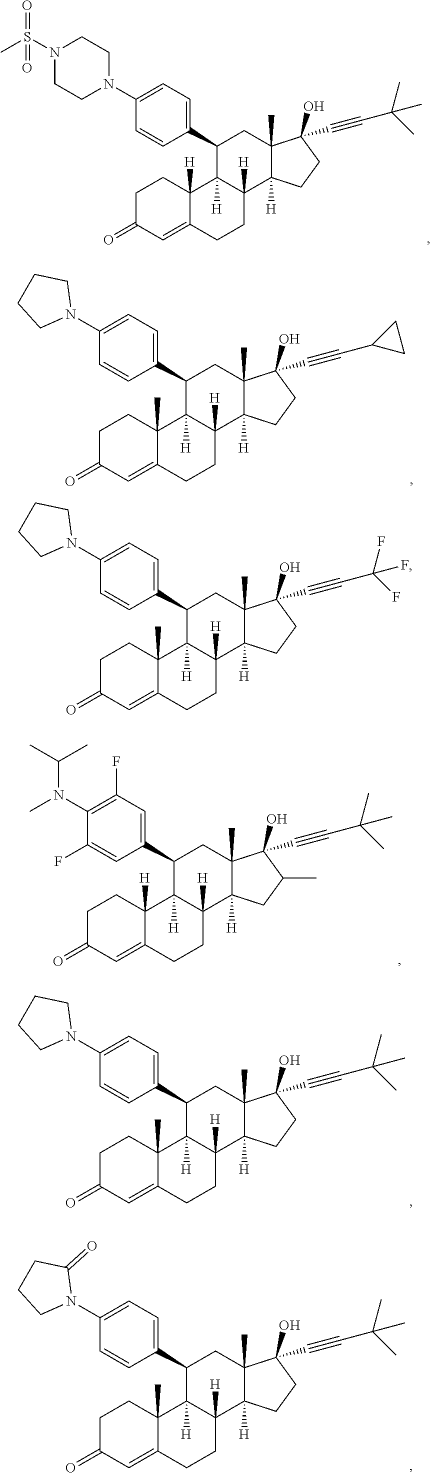 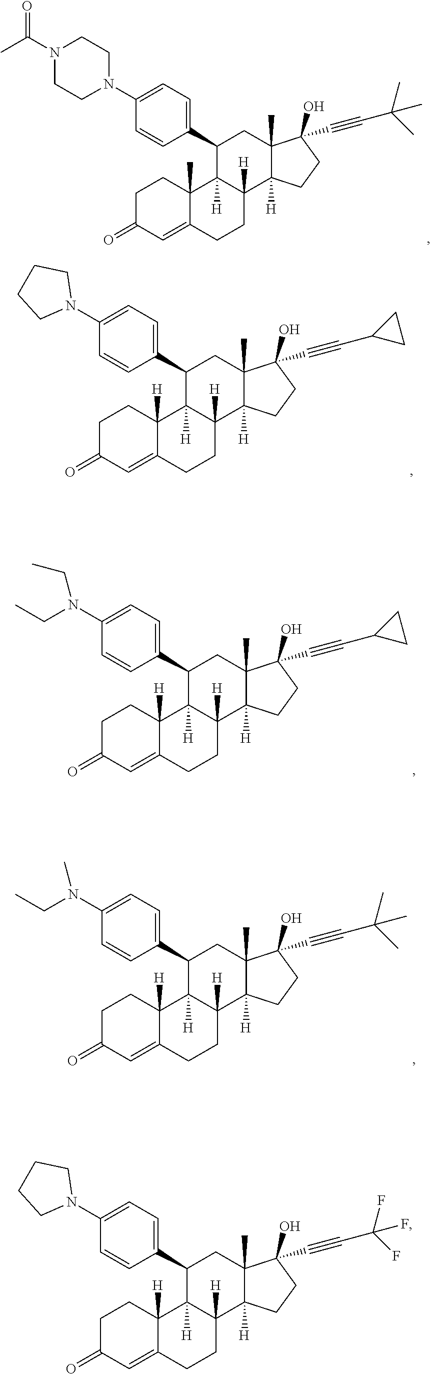 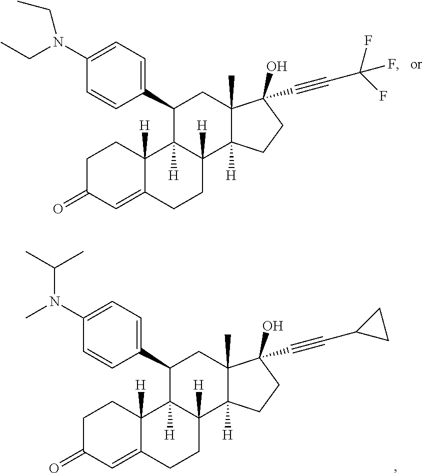
or a pharmaceutically acceptable salt thereof.

21. The lipid-based formulation of claim 1, wherein the compound is: 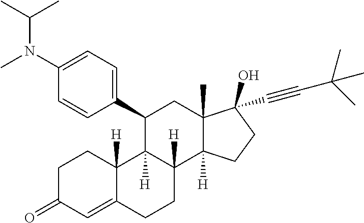
or a pharmaceutically acceptable salt thereof.

22. The lipid-based formulation of any one of claims 1-21, wherein the compound of Formula (I) is in the form of an HCl salt.

23. The lipid-based formulation of any one of claims 1-21, wherein the compound of Formula (I) is in the form of a free base.

24. The lipid-based formulation of any one of claims 1-23, wherein the lipid is propylene glycol monocaprylate (Capryol®), caprylic acid, capric acid, lauric acid, myristic acid, palmitic acid, stearic acid, oleic acid, ethyl oleate, soybean oil, glyceryl caprylate/caprate (Campul®) glyceryl behenate (Compritol® 888 ATO), glyceryl palmitostearate (Precirol® ATO 5), glyceryl monostearate (Geleol™), glyceryl monolinoleate (Maisine™ 35-1), glyceryl monooleate, (Peceol™), medium-chain triglycerides (Labrafac™ Lipophile WL1349), propylene glycol monolaurate (Lauroglycol™ 90), oleoyl macrogol-6 glycerides (Labrafil® M1944CS), polyglyceryl-3 dioleate (Plurol Oleique® CC 497), diethylene glycol monoethyl ether (Transcutol® HP), or any combinations thereof.

25. The lipid-based formulation of any one of claims 1-24, wherein the lipid-based formulation further comprises a surfactant.

26. The lipid-based formulation of claim 25, wherein the surfactant is macroglycerol ricinoleate (Kolliphor EL® or Cremophor EL®), caprylocaproyl polyoxyl-8 glyceride (Labrasol®), lauroyl polyoxyl-6 glycerides (Labrafil® M 2130 CS), lauroyl polyoxyl-32 glyceride (Gelucire® 44/14), polyethylene glycol monostearate (Gelucire® 48/16), polyoxyethylene hydrogenated castor oil 60 (HCO-60), polysorbate 80 (Tween®-80), polyethylene glycol sorbitan monolaurate (Tween®-20), polyoxyethylene sorbitan trioleate (Tween®-85), polyoxyethyelene glyceryl trioleate (tagot-TO), sorbitan monooleate (Span®-80), sorbitan monolaurate (Span®-20), or any combinations thereof.

27. The lipid-based formulation of any one of claims 1-26, wherein the lipid-based formulation further comprises an antioxidant.

28. The lipid-based formulation of claim 27, wherein the antioxidant is α-tocopherol, ascorbyl palmitate, butylated hydroxyanisole (BHA), butylated hydroxytoluene (BHT), sodium metabisulfite, potassium metabisulfite, propyl gallate, ascorbic acid, monothioglycerol, propionic acid, sodium ascorbate, sodium bisulfite, sodium sulfite, and cysteine (CYS), or any combinations thereof.

29. The lipid-based formulation of claim 27 or 28, wherein the antioxidant is α-tocopherol, ascorbyl palmitate, or any combinations thereof.

30. The lipid-based formulation of any one of claims 1-29, wherein the formulation is encapsulated.

31. The lipid-based formulation of claim 30, wherein the formulation is encapsulate is a gelatin capsule.

32. The lipid-based formulation of claim 30 or 31, wherein the amount of compound of Formula (I) or its pharmaceutically acceptable salt, in the capsule is between about 10 mg and about 100 mg.

33. The lipid-based formulation of any one of claims 30-32, wherein the amount of the compound of Formula (I) or its pharmaceutically acceptable salt, in the capsule is between about 20 mg and about 80 mg.

34. The lipid-based formulation of any one of claims 30-33, wherein the amount of the compound of Formula (I) or its pharmaceutically acceptable salt, in the capsule is between about 40 mg and about 60 mg.

35. The lipid-based formulation of any one of claims 30-33, wherein the amount of the compound of Formula (I) or its pharmaceutically acceptable salt, in the capsule is between about 60 mg and about 100 mg.

36. The lipid-based formulation of any one of claims 30-34, wherein the amount of the compound of Formula (I) or its pharmaceutically acceptable salt, in the capsule is about 50 mg.

37. The lipid-based formulation of any one of claims 30-33, wherein the amount of the compound of Formula (I) or its pharmaceutically acceptable salt, in the capsule is about 80 mg.

38. The lipid-based formulation of any one of claims 30-37, wherein the amount of lipid is between about 500 mg and about 900 mg.

39. The lipid-based formulation of any one of claims 30-38, wherein the amount of lipid is between about 700 mg and about 800 mg.

40. The lipid-based formulation of any one of claims 30-38, wherein the amount of lipid is between about 600 mg and about 700 mg.

41. The lipid-based formulation of any one of claims 30-40, wherein the amount of surfactant is between about 100 mg and about 500 mg.

42. The lipid-based formulation of any one of claims 30-41, wherein the amount of surfactant is between about 100 mg and about 200 mg.

43. The lipid-based formulation of any one of claims 1-42, wherein the lipid-based formulation comprises caprylic acid.

44. The lipid-based formulation of claim 43, wherein the amount of caprylic acid is about 750 mg.

45. The lipid-based formulation of claim 43, wherein the amount of caprylic acid is about 735 mg.

46. The lipid-based formulation of any one of claims 1-42, wherein the lipid-based formulation comprises propylene glycol monocaprylate (Capryol®) and macroglycerol ricinoleate (Kolliphor EL® or Cremophor EL®).

47. The lipid-based formulation of claim 46, wherein the amount of propylene glycol monocaprylate (Capryol®) is about 676 mg and the amount of macroglycerol ricinoleate (Kolliphor EL® or Cremophor EL®) is about 174 mg.

48. The lipid-based formulation of any one of claims 1-47, wherein the lipid-based formulation comprises α-tocopherol and ascorbyl palmitate.

49. The lipid-based formulation of claim 48, wherein the amount of α-tocopherol is about 4.1 mg and the amount of ascorbyl palmitate is about 0.25 mg.

50. The lipid-based formulation of any one of claims 1-49, wherein the lipid-based formulation forms a self-emulsifying drug delivery system (SEDDS) in an aqueous solution.

51. The lipid-based formulation of any one of claims 1-50, wherein the formulation is stable at about 5° C.±3° C. for at least 7 days.

52. The lipid-based formulation of any one of claims 1-50, wherein the formulation is stable at about 25° C.±5° C. for at least 7 days.

53. A crystalline compound of Formula (I), or a pharmaceutically acceptable salt thereof: 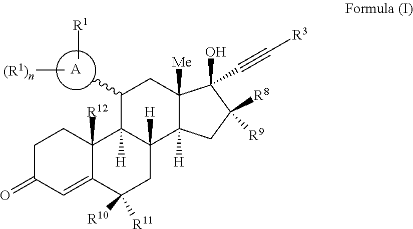
wherein
ring A is a heteroaryl or aryl;
R¹ is —NR^{4a}R^{5a};
each R² is independently —NR⁴R⁵, halo, —OR⁶, —OH, optionally substituted alkyl, or haloalkyl;
R³ is optionally substituted C₂₋₈ alkyl, halo, haloalkyl, optionally substituted cycloalkyl, optionally substituted cycloalkylalkyl, optionally substituted heterocycloalkyl, optionally substituted heterocycloalkylalkyl, optionally substituted heteroalkyl, optionally substituted aryl, optionally substituted heteroaryl, —Si(R⁶)₃, —OR⁶, or —S(O)₂R⁷;
R^{4a} is C₂₋₈ alkyl, optionally substituted cycloalkyl, optionally substituted aryl, optionally substituted heterocycloalkyl, or optionally substituted heteroaryl;
R^{5a} is —H, optionally substituted alkyl, or haloalkyl;
or R^{4a} and R^{5a} are taken together with the N atom to which they are attached to form an optionally substituted heterocycloalkyl;
R⁴ and R⁵ are each independently —H, optionally substituted alkyl, or haloalkyl;
or R⁴ and R⁵ are taken together with the N atom to which they are attached to form an optionally substituted heterocycloalkyl;
each R⁶ is independently optionally substituted alkyl or haloalkyl;
R⁷ is optionally substituted alkyl or haloalkyl;
R⁸ and R⁹ are each independently —H, optionally substituted alkyl, haloalkyl, or halo;
R¹⁰ and R¹¹ are each independently —H, optionally substituted alkyl, halo, or haloalkyl;
R¹² is hydrogen, optionally substituted alkyl, haloalkyl, hydroxy, or halo;
n is 0, 1, or 2.

54. The crystalline compound of claim 53, wherein the compound is: 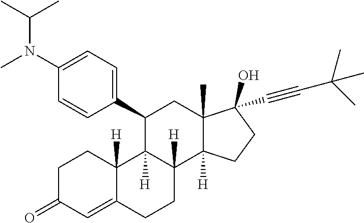
or a pharmaceutically acceptable salt thereof.

55. The crystalline compound of claim 54, wherein the crystalline form has an X-ray powder diffraction (XRPD) pattern substantially the same as shown in FIG. 1.

56. The crystalline compound of claim 54, wherein the crystalline form has an X-ray powder diffraction (XRPD) pattern with characteristic peaks at 7.2±0.1° 2-Theta, 15.7±0.1° 2-Theta, 16.6±0.1° 2-Theta, 18.3±0.1° 2-Theta, 19.3±0.1° 2-Theta and 20.1±0.1° 2-Theta.

57. The crystalline compound of claim 54, wherein the crystalline form has an X-ray powder diffraction (XRPD) pattern substantially the same as shown in FIG. 3.

58. The crystalline compound of claim 54, wherein the crystalline form has an X-ray powder diffraction (XRPD) pattern with characteristic peaks at 7.0±0.1° 2-Theta, 9.2±0.1° 2-Theta, 11.2±0.1° 2-Theta, 14.9±0.1° 2-Theta, 17.2±0.1° 2-Theta, and 19.2±0.1° 2-Theta.

59. A process for preparing 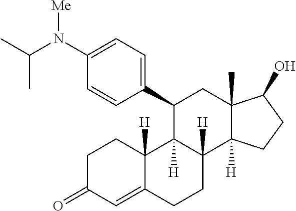
as outlined in Scheme 1:

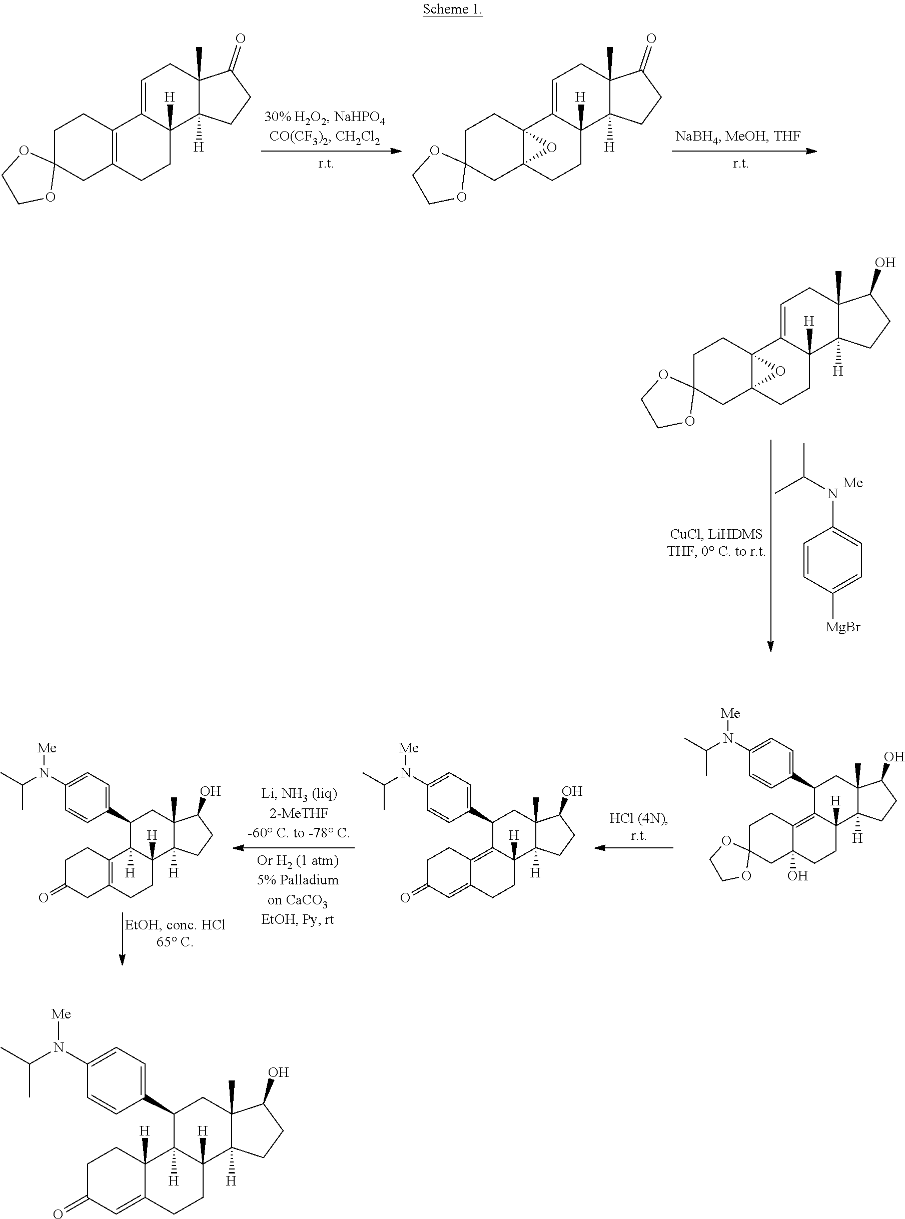

60. A process for preparing 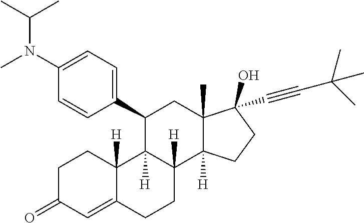
or a pharmaceutically acceptable salt thereof as outlined in Scheme 2:

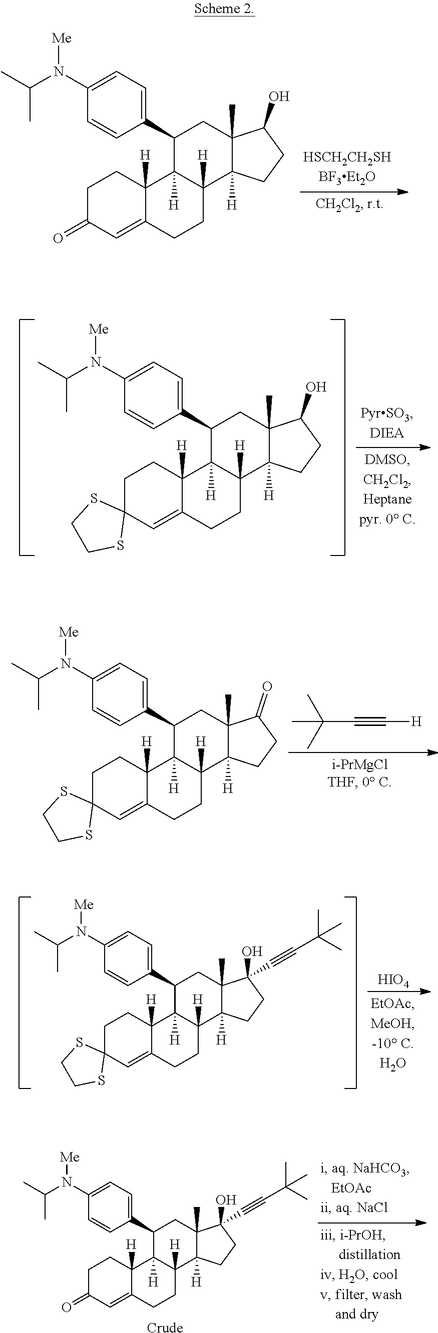

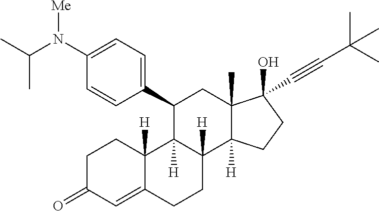

61. A method of treating non-small cell lung cancer, triple negative breast cancer, ovarian cancer, melanoma, pancreatic cancer, prostate cancer, castration resistant prostate cancer, renal cancer, melanoma, hepatocellular carcinoma, or bladder cancer, in a subject in need thereof; the method comprising administering a formulation selected from a lipid-based formulation of any one of claims 1-52 to the subject in need thereof.

62. The method of claim 61, wherein the formulation is administered orally.

63. The method of claim 61 or 62, wherein the dose of the compound of Formula (I) administered is between about 200 mg and about 800 mg.

64. The method of any one of claims 61-63, wherein the dose of the compound of Formula (I) administered is about 200 mg, about 300 mg, about 400 mg, about 500 mg, about 600 mg, about 700 mg, or about 800 mg.

65. The method of any one of claims 61-64, wherein the formulation is administered once a day.

66. The method of any one of claims 61-64, wherein the formulation is administered twice a day.

67. The method of any one of claims 61-66, wherein the formulation is administered in combination with an additional therapeutic agent.

68. The method of claim 67, wherein the additional therapeutic agent is an androgen signaling inhibitor, a chemotherapeutic agent, or immunotherapy.

69. The method of claim 68, wherein the androgen receptor signaling inhibitor is 3,3′-diindolylmethane (DIM), abiraterone acetate, apalutamide, darolutamide, bexlosteride, bicalutamide, dutasteride, epristeride, enzalutamide, finasteride, flutamide, izonsteride, ketoconazole, N-butylbenzene-sulfonamide, nilutamide, megestrol, steroidal antiandrogens, or turosteride.

70. The method of claim 68, wherein the chemotherapeutic agent is cisplatin, carboplatin, oxaliplatin, etoposide, vincristine, vinblastine, vinorelbine, paclitaxel, docetaxel, nab-paclitaxel, gemcitabine, capecitabine, 5-fluorouracil, doxorubicin, daunorubicin, epirubicin, cyclophosphamide, ifosfamide, camptothecin, topotecan, irinotecan, or pemetrexed.

71. The method of claim 68, wherein the chemotherapeutic agent is cisplatin, carboplatin, paclitaxel, docetaxel, nab-paclitaxel, gemcitabine, doxorubicin, camptothecin, topotecan, or pemetrexed.

72. The method of claim 68, wherein the immunotherapy is an anti-PD-L1 agent, an anti-PD1 agent, an anti-CTLA-4 agent, a CAR-T cell therapy, an IDO-1 inhibitor, or a cancer vaccine.

73. The method of any one of claims 67-72, wherein the formulation and the additional therapeutic agent are administered concurrently.

74. The method of any one of claims 67-72, wherein the formulation and the additional therapeutic agent are administered intermittently.

75. The method of any one of claims 67-72, wherein the formulation and the additional therapeutic agent are administered in a 21-day therapeutic cycle.

76. The method of any one of claims 67-72, wherein the formulation is administered daily and the additional therapeutic agent is administered on day 1 of a 21-day cycle.

77. The method of any one of claims 67-72, wherein the formulation is administered on days 1-7 and the additional therapeutic agent is administered on day 1 of a 21-day cycle.

78. The method of any one of claims 67-72, wherein the formulation is administered daily and the additional therapeutic agent is administered on day 1, day 8, and day 15 of a 21-day cycle.

79. The method of any one of claims 67-72, wherein the formulation is administered on days 1-7 and the additional therapeutic agent is administered on day 1, day 8, and day 15 of a 21-day cycle.

80. The method of any one of claims 67-72, wherein the formulation is administered for 3 days of each week per 3 week cycle.

81. The method of any one of claims 67-72, wherein the formulation is administered for 4 days of each week per 3 week cycle.

82. The method of any one of claims 67-72, wherein the formulation is administered for 5 days of each week per 3 week cycle.

83. The method of any one of claims 67-72, wherein the formulation is administered for 6 days of each week per 3 week cycle.

84. The method of any one of claims 80-83, wherein the additional therapeutic agent is administered on day 1 of a 21-day cycle.

85. The method of any one of claims 80-83, wherein the additional therapeutic agent is administered on day 1, day 8, and day 15 of a 21-day cycle.

86. The method of any one of claims 75-85, wherein the formulation and additional therapeutic agent are administered for multiple cycles.
